(19) United States
(12) Patent Application Publication
(10) Pub. No.: US 2022/0016136 A1
(43) Pub. Date: Jan. 20, 2022

(54) COMPOSITIONS AND METHODS FOR THE TREATMENT OF LIVER DISORDERS

(71) Applicant: Viking Therapeutics, Inc., San Diego, CA (US)

(72) Inventors: Brian Lian, Rancho Santa Fe, CA (US); Hiroko Masamune, San Diego, CA (US)

(21) Appl. No.: 17/311,014

(22) PCT Filed: Dec. 4, 2019
(86) PCT No.: PCT/US2019/064548

§ 371 Date: Jun. 4, 2021

Related U.S. Application Data
(60) Provisional application No. 62/775,799, filed on Dec. 5, 2018.
(60) Provisional application No. 62/821,303, filed on Mar. 20, 2019.

(51) Int. Cl. A61K 31/661; A61K 31/366; A61K 31/192; A61K 31/662; A61K 31/664; A61K 31/66; A61K 31/404; A61K 31/40; A61K 31/501; A61K 31/216; A61K 31/575; A61K 31/397; A61K 31/46; A61K 31/422; A61K 31/42; A61K 31/55; A61K 31/4184; A61K 31/554; A61K 31/495; A61K 31/4412; A61K 31/415; A61K 31/41; A61K 31/5377; A61K 31/341; A61K 31/4192; A61K 31/381; A61K 31/4709; A61K 31/498; A61K 31/553; A61K 31/4245; A61K 31/435; A61K 31/506; A61K 31/4353; A61K 31/551; A61K 38/26; A61K 38/28; A61K 38/18; A61K 45/06; A61P 1/16

(52) U.S. Cl. CPC A61K 31/661; A61P 1/16; A61K 31/192; A61K 31/662; A61K 31/664; A61K 31/66; A61K 31/404; A61K 31/40; A61K 31/501; A61K 31/216; A61K 31/575; A61K 31/397; A61K 31/46; A61K 31/422; A61K 31/42; A61K 31/55; A61K 31/4184; A61K 31/554; A61K 31/495; A61K 31/4412; A61K 31/415; A61K 31/41; A61K 31/5377; A61K 31/341; A61K 31/4192; A61K 31/381; A61K 31/4709; A61K 31/498; A61K 31/553; A61K 31/4245; A61K 31/435; A61K 31/506; A61K 31/4353; A61K 31/551; A61K 38/26; A61K 38/28; A61K 38/1825; A61K 45/06; A61K 31/366

(57) ABSTRACT

The present disclosure is directed toward the use of thyroid receptor agonists of pharmaceutically acceptable salts thereof, in combination with a second pharmaceutical agent for preventing, treating, or ameliorating fatty liver diseases such as steatosis, non-alcoholic fatty liver disease, and non-alcoholic steatohepatitis.

### BACKGROUND

### Field

The present disclosure relates generally to the field of treatments for fatty liver diseases and more specifically to the field of small molecule drugs for the treatment of non-alcoholic steatohepatitis.

### Description of the Related Art

Thyroid hormones (TH) are synthesized in the thyroid in response to thyroid stimulating hormone (TSH), which is secreted by the pituitary gland in response to various stimulants (e.g., thyrotropin-releasing hormone (TRH) from the hypothalamus). Thyroid hormones are iodinated O-aryl tyrosine analogues excreted into the circulation primarily as 3,3′,5,5′-tetraiodothyronine (T4). T4 is rapidly deiodinated in local tissues by thyroxine 5′-deiodinase to 3,3′,5′-triiodothyronine (T3), which is the most potent TH. T3 is metabolized to inactive metabolites via a variety of pathways, including pathways involving deiodination, glucuronidation, sulfation, deamination, and decarboxylation. Most of the circulating T4 and T3 is eliminated through the liver.

The biological activity of THs is mediated largely through thyroid hormone receptors (TRs). TRs belong to the nuclear receptor superfamily, which, along with its common partner, the retinoid X receptor, form heterodimers that act as ligand-inducible transcription factors. Like other nuclear receptors, TRs have a ligand binding domain and a DNA binding domain and regulate gene expression through ligand-dependent interactions with DNA response elements (thyroid response elements, TREs). Currently, the literature shows that TRs are encoded by two distinct genes (TRα and TRβ), which produce several isoforms through alternative splicing (Williams, Mol. Cell. Biol. 20(22):8329-42 (2000); Nagaya et al., Biochem. Biophys. Res. Commun. 226(2):426-30 (1996)). The major isoforms that have so far been identified are TRα-1, TRα-2, TRβ-1 and TRβ-2. TRα-1 is ubiquitously expressed in the rat with highest expression in skeletal muscle and brown fat. TRβ-1 is also ubiquitously expressed with highest expression in the liver, brain and kidney. TRβ-2 is expressed in the anterior pituitary gland and specific regions of the hypothalamus as well as the developing brain and inner ear. In the rat and mouse liver, TRβ-1 is the predominant isoform (80%). The TR isoforms found in human and rat are highly homologous with respect to their amino acid sequences which suggest that each serves a specialized function.

TH's affect the growth, metabolism and the physiological function of nearly all organs. TH's lower serum cholesterol and triglycerides. However, side effects of TH action include cardiac arrhythmia, bone loss, nervousness, and anxiety. Accordingly, there is a need for novel therapies that can be used to modulate cholesterol levels and triglycerides and control other metabolic disorders while minimizing undesired effects.

### SUMMARY

Some embodiments disclosed herein relate to a method of preventing, treating, or ameliorating one or more fatty liver diseases in a subject in need thereof comprising administering to said subject in need thereof at least one TR-β agonist compound in combination with a second pharmaceutical agent.

In some embodiments, the TR-β agonist is a compound of Formula I:

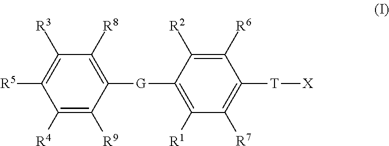

or a pharmaceutically acceptable salt thereof, wherein:

G is selected from the group consisting of —O—, —S—, —S(═O)—, —S(═O)₂—, —Se—, —CH₂—, —CF₂—, —CHF—, —C(O)—, —CH(OH)—, —CH(C₁-C₄ alkyl)-, —CH(C₁-C₄ alkoxy)-, —C(═CH₂)—, —NH—, and —N(C₁-C₄ alkyl)-;

T is selected from the group consisting of —(CR^{a}₂)ₖ—, —CR^{b}═CR^{b}—(CR^{a}₂)ₙ—, —(CR^{a}₂)ₙ—CR^{b}═CR^{b}—, —(CR^{a}₂)—CR^{b}═CR^{b}—(CR^{a}₂)—, —O(CR^{b}₂)(CR^{a}₂)ₙ—, —S(CR^{b}₂)(CR^{a}₂)ₙ—, N(R^{c})(CR^{b}₂)(CR^{a}₂)ₙ—, N(R^{b})C(O)(CR^{a}₂)ₙ, —C(O)(CR^{a}₂)ₘ—, —(CR^{a}₂)ₘC(O)—, —(CR^{a}₂)C(O)(CR^{a}₂)ₙ, —(CR^{a}₂)ₙC(O)(CR^{a}₂)—, and —C(O)NH(CR^{b}₂)(CR^{a}₂)ₚ—;

k is an integer from 1-4;

m is an integer from 0-3;

n is an integer from 0-2;

p is an integer from 0-1;

each R^{a} is independently selected from the group consisting of hydrogen, optionally substituted —C₁-C₄ alkyl, halogen, —OH, optionally substituted —O—C₁-C₄ alkyl, —OCF₃, optionally substituted —S—C₁-C₄ alkyl, —NR^{b}R^{c}, optionally substituted —C₂-C₄ alkenyl, and optionally substituted —C₂-C₄ alkynyl; with the proviso that when one R^{a} is attached to C through an O, S, or N atom, then the other R^{a} attached to the same C is a hydrogen, or attached via a carbon atom;

each R^{b} is independently selected from the group consisting of hydrogen and optionally substituted —C₁-C₄ alkyl;

each R^{c} is independently selected from the group consisting of hydrogen and optionally substituted —C₁-C₄ alkyl, optionally substituted —C(O)—C₁-C₄ alkyl, and —C(O)H;

R¹, and R² are each independently selected from the group consisting of halogen, optionally substituted —C₁-C₄ alkyl, optionally substituted —S—C₁-C₃ alkyl, optionally substituted —C₂-C₄ alkenyl, optionally substituted —C₂-C₄ alkynyl, —CF₃, —OCF₃, optionally substituted-O—C₁-C₃ alkyl, and cyano;

R⁶, R⁷, R⁸, and R⁹ are each independently selected from the group consisting of are each independently selected from the group consisting of hydrogen, halogen, optionally substituted —C C₁-C₄ alkyl, optionally substituted —S—C₁-C₃ alkyl, optionally substituted —C₂-C₄ alkenyl, optionally substituted —C₂-C₄ alkynyl, —CF₃, —OCF₃, optionally substituted-O—C₁-C₃ alkyl, and cyano; or R⁶ and T are taken together along with the carbons they are attached to form a ring of 5 to 6 atoms including 0 to 2 heteroatoms independently selected from —NR¹—, —O—, and —S—, with the proviso that when there are 2 heteroatoms in the ring and both heteroatoms are different than nitrogen then both heteroatoms have to be separated by at least one carbon atom; and X is attached to this ring by a direct bond to a ring carbon, or by —(CR^{a}₂)— or —C(O)— bonded to a ring carbon or a ring nitrogen;

R¹ is selected from the group consisting of hydrogen, —C(O)C₁-C₄ alkyl, —C₁-C₄ alkyl, and —C₁-C₄-aryl;

R³ and R⁴ are independently selected from the group consisting of hydrogen, halogen, —CF₃, —OCF₃, cyano, optionally substituted —C₁-C₁₂ alkyl, optionally substituted —C₂-C₁₂ alkenyl, optionally substituted —C₂-C₁₂ alkynyl, —SR^{d}, —S(═O)R^{e}, —S(═O)₂R^{e}, —S(═O)₂NR^{f}R^{g}, —C(O)OR^{h}, —C(O)R^{e}, —N(R^{b})C(O)NR^{f}R^{g}, —N(R^{b})S(═O)₂R^{e}, —N(R^{b})S(═O)₂NR^{f}R^{g}, and —NR^{f}R^{g};

each R^{d} is selected from the group consisting of optionally substituted —C₁-C₁₂ alkyl, optionally substituted —C₂-C₁₂ alkenyl, optionally substituted —C₂-C₁₂ alkynyl, optionally substituted —(CR^{b}₂)ₙ aryl, optionally substituted —(CR^{b}₂)ₙ cycloalkyl, optionally substituted —(CR^{b}₂)ₙ heterocycloalkyl, and —C(O)NR^{f}R^{g};

each R^{e} is selected from the group consisting of optionally substituted —C₁-C₁₂ alkyl, optionally substituted —C₂-C₁₂ alkenyl, optionally substituted —C₂-C₁₂ alkynyl, optionally substituted —(CR^{a}₂)ₙ aryl, optionally substituted —(CR^{a}₂)ₙ cycloalkyl, and optionally substituted —(CR^{a}₂)ₙ heterocycloalkyl;

R^{f} and R^{g} are each independently selected from the group consisting of hydrogen, optionally substituted —C₁-C₁₂ alkyl, optionally substituted —C₂-C₁₂ alkenyl, optionally substituted —C₂-C₁₂ alkynyl, optionally substituted —(CR^{b}₂)ₙ aryl, optionally substituted —(CR^{b}₂)ₙ cycloalkyl, and optionally substituted —(CR^{b}₂)ₙ heterocycloalkyl, or R^{f} and R^{g} may together form an optionally substituted heterocyclic ring, which may contain a second heterogroup selected from the group consisting of O, NR^{C}, and S, wherein said optionally substituted heterocyclic ring may be substituted with 0-4 substituents selected from the group consisting of optionally substituted —C₁-C₄ alkyl, —OR^{b}, oxo, cyano, —CF₃, optionally substituted phenyl, and —C(O)OR^{h};

each R^{h} is selected from the group consisting of optionally substituted —C₁-C₁₂ alkyl, optionally substituted —C₂-C₁₂ alkenyl, optionally substituted —C₂-C₁₂ alkynyl, optionally substituted —(CR^{b}₂)ₙ aryl, optionally substituted —(CR^{b}₂)ₙ cycloalkyl, and optionally substituted —(CR^{b}₂)ₙ heterocycloalkyl;

R⁵ is selected from the group consisting of —OH, optionally substituted —OC₁-C₆, alkyl, OC(O)R^{e}, —OC(O)OR^{h}, —F, —NHC(O)R^{e}, —NHS(═O)R^{e}, —NHS(═O)₂R^{e}, —NHC(═S)NH(R^{h}), and —NHC(O)NH(R^{h});

X is P(O)YR¹¹Y′R¹¹;

Y and Y′ are each independently selected from the group consisting of —O—, and —NR^{v}—; when Y and Y′ are —O—, R¹¹ attached to —O— is independently selected from the group consisting of —H, alkyl, optionally substituted aryl, optionally substituted heterocycloalkyl, optionally substituted CH₂-heterocycloakyl wherein the cyclic moiety contains a carbonate or thiocarbonate, optionally substituted -alkylaryl, —C(R^{z})₂OC(O)NR^{z}₂, —NR^{z}—C(O)—R^{y}, —C(R^{z})₂—OC(O)R^{y}, —C(R^{z})₂—O—C(O)OR^{y}, —C(R^{z})₂OC(O)SR^{y}, -alkyl-S—C(O)R^{y}, -alkyl-S—S-alkylhydroxy, and -alkyl-S—S—S-alkylhydroxy;

when Y and Y′ are —NR^{v}—, then R¹¹ attached to —NR^{v}— is independently selected from the group consisting of —H, —[C(R^{z})₂]q-COOR^{y}, —C(R^{x})₂COOR^{Y}, —[C(R^{z})₂]_{q}—C(O)SR^{y}, and -cycloalkylene-COOR^{y};

when Y is —O— and Y′ is NR^{v}, then R¹¹ attached to —O— is independently selected from the group consisting of —H, alkyl, optionally substituted aryl, optionally substituted heterocycloalkyl, optionally substituted CH₂-heterocycloakyl wherein the cyclic moiety contains a carbonate or thiocarbonate, optionally substituted -alkylaryl, —C(R^{z})₂OC(O)NR^{z}₂, —NR^{z}—C(O)—R^{y}, —C(R^{z})₂—OC(O)R^{y}, —C(R^{z})₂—O—C(O)OR^{y}, —C(R^{z})₂OC(O)SR^{y}, -alkyl-S—C(O)R^{y}, -alkyl-S—S-alkylhydroxy, and -alkyl-S—S—S-alkylhydroxy; and R¹¹ attached to —NR^{v}— is independently selected from the group consisting of H, —[C(R^{z})₂]_{q}—COOR^{y}, —C(R^{x})₂COOR^{y}, —[C(R^{z})₂]_{q}—C(O)SR^{y}, and -cycloalkylene-COOR^{y};

or when Y and Y′ are independently selected from —O— and NR^{v}, then together R¹¹ and R¹¹ are -alkyl-S—S-alkyl- to form a cyclic group, or together R¹¹ and R¹¹ are the group:

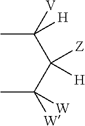

wherein:

V, W, and W′ are independently selected from the group consisting of hydrogen, optionally substituted alkyl, optionally substituted aralkyl, heterocycloalkyl, aryl, substituted aryl, heteroaryl, substituted heteroaryl, optionally substituted 1-alkenyl, and optionally substituted 1-alkynyl;

or together V and Z are connected via an additional 3-5 atoms to form a cyclic group containing 5-7 atoms, wherein 0-1 atoms are heteroatoms and the remaining atoms are carbon, substituted with hydroxy, acyloxy, alkylthiocarbonyloxy, alkoxycarbonyloxy, or aryloxycarbonyloxy attached to a carbon atom that is three atoms from both Y groups attached to the phosphorus;

or together V and Z are connected via an additional 3-5 atoms to form a cyclic group, wherein 0-1 atoms are heteroatoms and the remaining atoms are carbon, that is fused to an aryl group at the beta and gamma position to the Y attached to the phosphorus;

or together V and W are connected via an additional 3 carbon atoms to form an optionally substituted cyclic group containing 6 carbon atoms and substituted with one substituent selected from the group consisting of hydroxy, acyloxy, alkoxycarbonyloxy, alkylthiocarbonyloxy, and aryloxycarbonyloxy, attached to one of said carbon atoms that is three atoms from a Y attached to the phosphorus;

or together Z and W are connected via an additional 3-5 atoms to form a cyclic group, wherein 0-1 atoms are heteroatoms and the remaining atoms are carbon, and V must be aryl, substituted aryl, heteroaryl, or substituted heteroaryl;

or together W and W′ are connected via an additional 2-5 atoms to form a cyclic group, wherein 0-2 atoms are heteroatoms and the remaining atoms are carbon, and V must be aryl, substituted aryl, heteroaryl, or substituted heteroaryl;

Z is selected from the group consisting of —CHR^{z}OH, —CHR^{z}OC(O)R^{y}, —CHR^{z}OC(S)R^{y}, —CHR^{z}OC(S)OR^{y}, —CHR^{z}OC(O)SR^{y}, —CHR^{z}OCO₂R^{y}, —OR^{z}, —SR^{z}, —CHR^{z}N₃, —CH₂-aryl, —CH(aryl)OH, —CH(CH═CR^{z}₂)OH, —CH(C≡CR^{z})OH, —R^{z}, —NR^{z}₂, —OCOR^{y}, —OCO₂R^{y}, —SCOR^{y}, —SCO₂R^{y}, —NHCOR^{z}, —NHCO₂R^{y}, —CH₂NH-aryl, —(CH₂)q-OR^{z}, and —(CH₂)q-SR^{z};

q is an integer 2 or 3;

each R^{z} is selected from the group consisting of R^{y} and —H;

each R^{y} is selected from the group consisting of alkyl, aryl, heterocycloalkyl, and aralkyl;

each R^{x} is independently selected from the group consisting of —H, and alkyl, or together R^{x} and R^{x} form a cyclic alkyl group; and

each R^{v} is selected from the group consisting of —H, lower alkyl, acyloxyalkyl, alkoxycarbonyloxyalkyl, and lower acyl.

In some embodiments, the TR-β agonist is a compound having the structure of Formula (A)

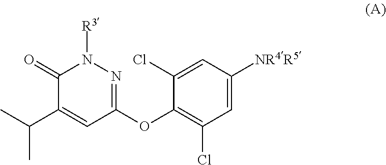

wherein

R^{3′} is H or CH₂R^{a}, in which R^{a} is hydroxyl, O-linked amino acid, —OP(O)(OH)₂ or OC(O)R^{b}, R^{b} being lower alkyl, alkoxy, alkyl acid, cycloalkyl, aryl, heteroaryl, or —(CH₂)_{n′}-heteroaryl and n′ being 0 or 1;

R^{4′} is H, and R^{5′} is CH₂COOH, C(O)CO₂H, or an ester or amide thereof, or R^{4′} and R^{5′} together are —N═C(R^{c′})—C—(O)—NH—C(O)—; in which R^{c′} is H or cyano;

or pharmaceutically acceptable salts thereof.

In some embodiments, the TR-β agonist is

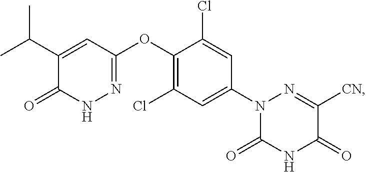 [SMILES: CC(C)c1cc(Oc2c(Cl)cc(-n3nc(C#N)c(=O)[nH]c3=O)cc2Cl)n[nH]c1=O]

or a pharmaceutically acceptable salt thereof.

Some embodiments disclosed herein relate to a method of preventing, treating, or ameliorating one or more fatty liver diseases in a subject in need thereof comprising administering to said subject in need thereof at least one compound selected from the group consisting of:

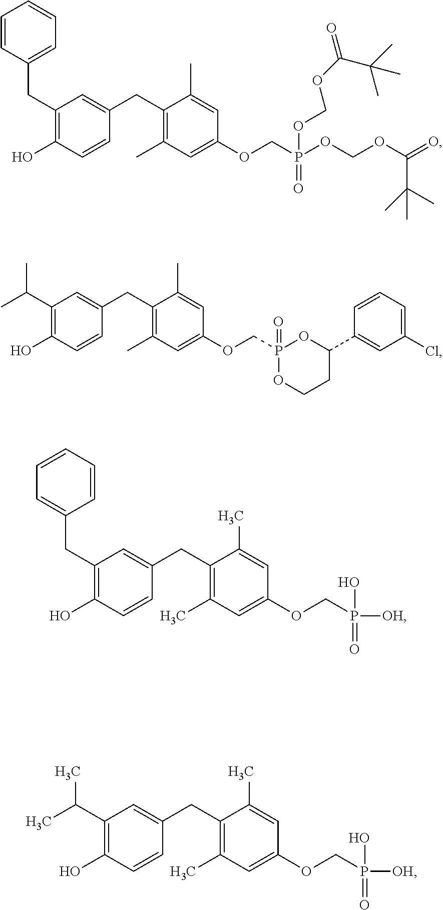

or pharmaceutically acceptable salts thereof to a subject in need thereof, in combination with a second pharmaceutical agent.

In some embodiments, the second pharmaceutical agent may be peroxisome proliferator-activated receptor (PPAR) modulator, a bile acid receptor modulator, an anti-inflammatory compound, an antifibrotic compound, a GLP-1 (Glucagon-like peptide-1) agonist, a metabolic modulator, or any combination of the foregoing.

In some embodiments, the second pharmaceutical agent may be a PPAR modulator. In some embodiments, the PPAR modulator may be:

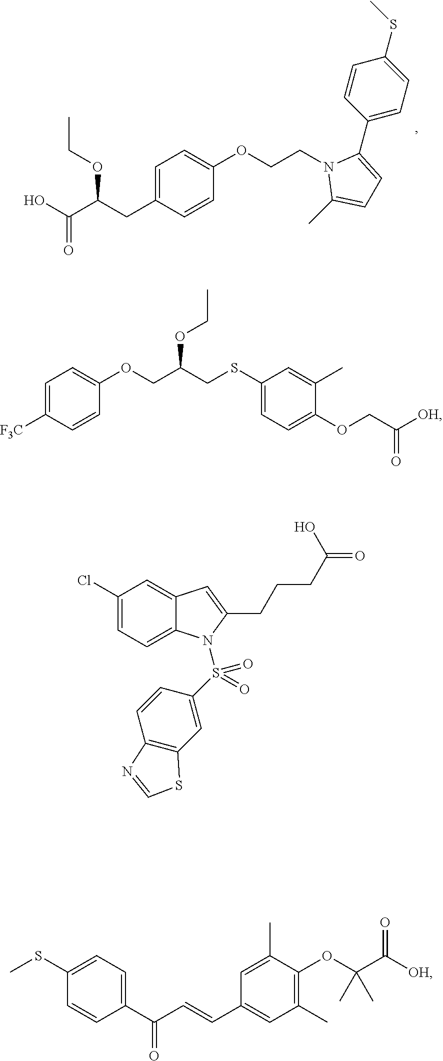 [SMILES: CCO[C@@H](Cc1ccc(OCCn2c(C)ccc2-c2ccc(SC)cc2)cc1)C(=O)O ; CCO[C@H](COc1ccc(C(F)(F)F)cc1)CSc1ccc(OCC(=O)O)c(C)c1 ; O=C(O)CCCc1cc2cc(Cl)ccc2n1S(=O)(=O)c1ccc2ncsc2c1 ; CSc1ccc(C(=O)/C=C/c2cc(C)c(OC(C)(C)C(=O)O)c(C)c2)cc1]

or a pharmaceutically acceptable salt thereof.

In some embodiments, the second pharmaceutical agent be a fibric acid derivative. In some embodiments, the fibric acid derivative may be fenofibrate, gemfibrozil, fenofibric acid, or clofibrate, or a pharmaceutically acceptable salt thereof.

In some embodiments, the second pharmaceutical agent may be a bile acid receptor modulator, such as the farnesoid X receptor (FXR). In some embodiments, the bile acid receptor modulator may be:

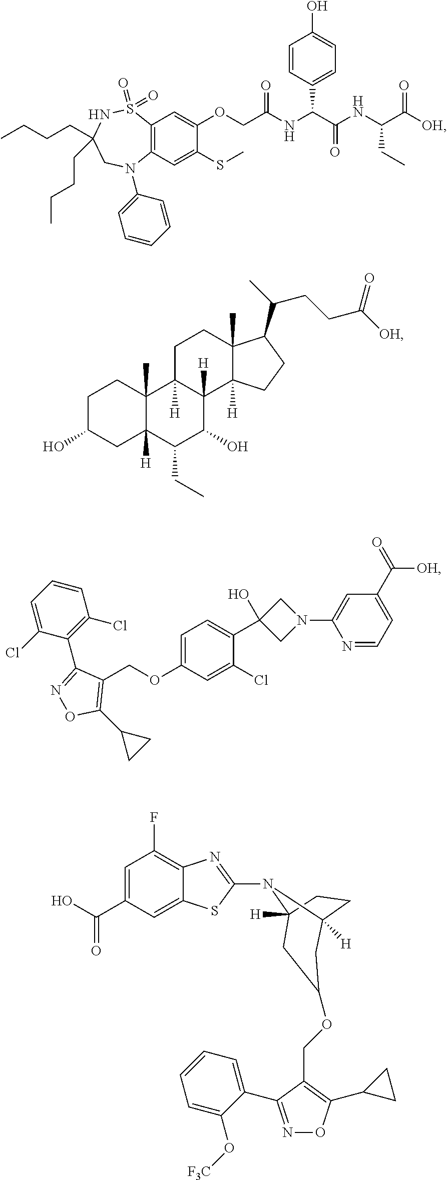

or a pharmaceutically acceptable salt thereof.

In some embodiments, the second pharmaceutical agent may be an anti-inflammatory compound. In some embodiments, the anti-inflammatory compound may be an inhibitor of apoptosis signal-regulating kinase 1 (ASK1). In some embodiments, the anti-inflammatory compound may be:

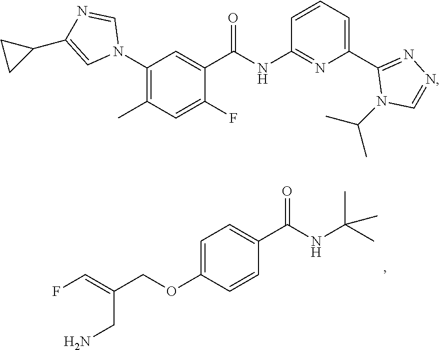

poly-clonal or mono-clonal anti-LPS immunoglobulins such as 1MM-124E,
or pharmaceutically acceptable salts thereof.

In some embodiments, the second pharmaceutical agent may be an anti-fibrotic compound. In some embodiments, the anti-fibrotic compound may be

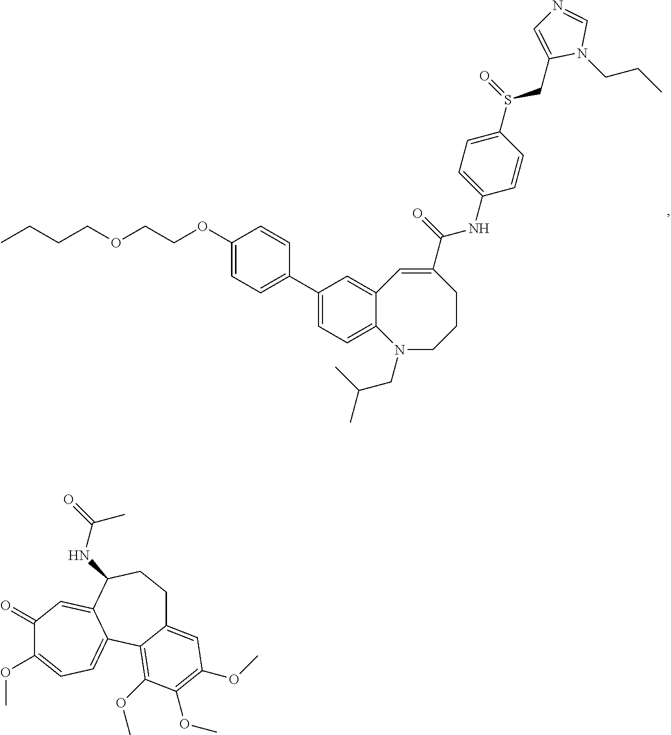 [SMILES: CCCCOCCOc1ccc(-c2ccc3c(c2)/C=C(/C(=O)Nc2ccc([S@@](=O)Cc4cncn4CCC)cc2)CCCN3CC(C)C)cc1 ; COc1cc2c(c(OC)c1OC)-c1ccc(OC)c(=O)cc1[C@@H](NC(C)=O)CC2]

or a pharmaceutically acceptable salt thereof.

In some embodiments, the second pharmaceutical agent may be a GLP-1 agonist. In some embodiments, the GLP-1 agonist is selected from dulaglutide, exenatide, liraglutide, albiglutide, lixisenatide, semaglutide, and insulin glargine. In some embodiments, the GLP-1 agonist is

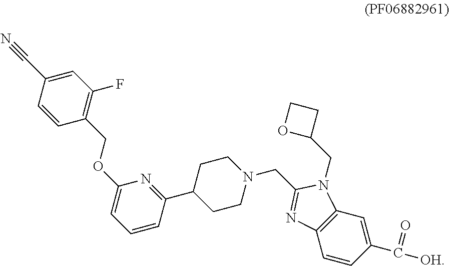 [SMILES: N#Cc1ccc(COc2cccc(C3CCN(Cc4nc5ccc(C(=O)O)cc5n4CC4CCO4)CC3)n2)c(F)c1]

In some embodiments, the second pharmaceutical agent has dual activity at GLP-1 and glucagon receptors (e.g., a dual acting GLP-1/glucagon agonist. In some embodiments, the second pharmaceutical agent has dual activity at GLP-1 and glucose-dependent insulinotropic polypeptide (GIP) (e.g., a dual acting GLP-1/GIP agonist).

In some embodiments, the second pharmaceutical agent is a metabolic modulator. In some embodiments, the metabolic modulator is a thyroid hormone receptor agonist, a selective androgen receptor modulator, a mitochondrial membrane transport protein modulator, a selective estrogen receptor modulator, an inhibitor of stearoyl-CoA desaturase 1 (SCD1), an inhibitor of dipeptidyl peptidase 4 (DPP-4), an inhibitor of sodium glucose cotransporters 1 and/or 2 (SGLT1, SGLT2, or dual SGLT1/SGLT2 inhibitors), recombinant fibroblast growth factor 19 (FGF19) or engineered analogs, or recombinant fibroblast growth factor 21 (FGF21) and pegylated variants. In some embodiments, the metabolic modulator is:

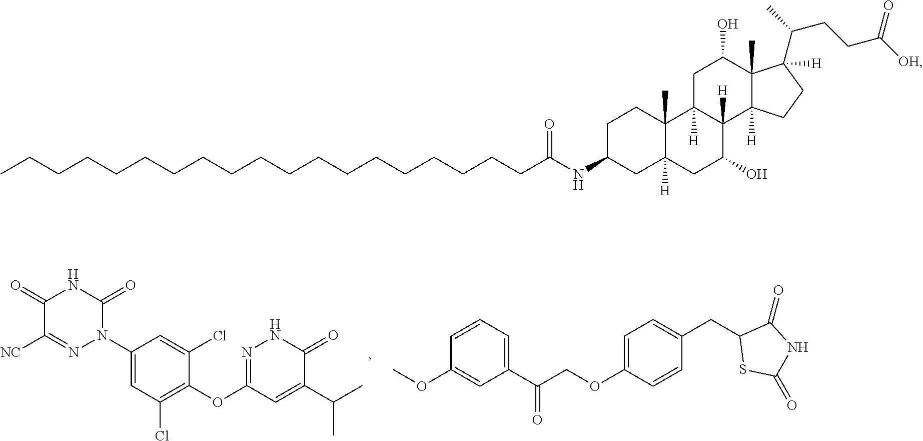

or a pharmaceutically acceptable salt thereof.

In some embodiments, the second pharmaceutical agent may be a fish oil derivative. In some embodiments, the fish oil derivative may be an omega-3-fatty acid alkyl ester or an omega-3-fatty acid trigylyceride. In some embodiments, the omega-3-fatty acid alkyl ester may be an omega-3-fatty acid ethyl ester. In some embodiments, the omega-3-fatty acid ethyl ester may be ethyl (5Z,8Z,11Z,14Z,17Z)-eicosa-5,8,11,14,17-pentaenoate, ethyl (4Z,7Z,10Z, 13Z, 16Z, 19Z)-docosa-4,7,10,13,16,19-hexaenoate, ethyl (7Z,10Z,13Z,16Z,19Z)-docosapentaenoate, ethyl hexadecatrienoate, α-linolenic acid ethyl ester, ethyl (6Z,9Z,12Z,15Z)-6,9,12,15-octadecatetraenoate, ethyl eicosatrienoate, ethyl eicosatetraenoate, ethyl heneicosapentaenoate, ethyl icosapentaenoate, ethyl heneicosapentaenoate, ethyl tetracosapentaenoate, or nisinic acid ethyl ester.

In some embodiments, the second pharmaceutical agent is an inhibitor of diacylglycerol acyltransferase (DGAT), such as compounds described in International Publication No. WO 2010/108051, which is incorporated herein by reference in its entirety.

In some embodiments, the compounds and second pharmaceutical agents provided herein may be used in a method of preventing, treating, or ameliorating one or more fatty liver diseases in a subject in need thereof. In some embodiments, the fatty liver disease can be of steatosis, non-alcoholic fatty liver disease (NAFLD), non-alcoholic steatohepatitis (NASH) and any combination of the foregoing.

Some embodiments provided herein the compounds and second pharmaceutical agents provided herein may be formulated into pharmaceutical compositions. In some embodiments, the compositions may formulated for oral, intravenous, intraarterial, intestinal, rectal, vaginal, nasal, pulmonary, topical, intradermal, transdermal, transbuccal, translingual, sublingual, or opthalmic administration, or any combination thereof.

In some embodiments, the compounds and second pharmaceutical agents provided herein may be administered sequentially. In some embodiments, the compounds and second pharmaceutical agents provided herein simultaneously. In some embodiments, the administration of the compounds and second pharmaceutical agents provided herein may result in the prevention, treatment, or amelioration, of a fibrosis, fibrotic condition, or fibrotic symptom in a subject. In some embodiments, the administration of the compounds and second pharmaceutical agents provided herein may result in the reduction in the amount of extracellular matrix proteins present in one or more tissues of a subject. In some embodiments, the administration of the compounds and second pharmaceutical agents provided herein may result in the reduction in the amount of collagen present in one or more tissues of a subject. In some embodiments, the administration of the compounds and second pharmaceutical agents provided herein may result in the reduction in the amount of Type I, Type Ia, or Type III collagen present in one or more tissues of the subject.

Some embodiments disclosed herein relate to a pharmaceutical composition comprising one or more compounds of Formula (I) and one or more second pharmaceutical agents.

### BRIEF DESCRIPTION OF THE DRAWINGS

FIG. 1 shows total liver hydroxyproline content in mice following 8 weeks of treatment with vehicle, Compound 2, Obeticholic Acid (OCA), Cenicriviroc (CVC), Elafibranor (ELA), Compound 2 and Obeticholic Acid (OCA), Compound 2 and Cenicriviroc (CVC), or Compound 2 and Elafibranor (ELA).

FIG. 2 shows representative images of liver stained with hematoxylin and eosin (HE staining at the end of the treatment period following 8 weeks of treatment vehicle, Compound 2, Obeticholic Acid (OCA), Cenicriviroc (CVC), Elafibranor (ELA), Compound 2 and Obeticholic Acid (OCA), Compound 2 and Cenicriviroc (CVC), or Compound 2 and Elafibranor (ELA) (magnification 20×, scale bar=100 μm).

FIG. 3 is a summary of Ballooning degeneration scores pre- and post-study biopsies following 8 weeks of treatment vehicle, Compound 2, Obeticholic Acid (OCA), Cenicriviroc (CVC), Elafibranor (ELA), Compound 2 and Obeticholic Acid (OCA), Compound 2 and Cenicriviroc (CVC), or Compound 2 and Elafibranor (ELA).

FIG. 4 shows relative liver weight as a percentage of body weight post-biopsy following 8 weeks of treatment vehicle, Compound 2, Obeticholic Acid (OCA), Cenicriviroc (CVC), Elafibranor (ELA), Compound 2 and Obeticholic Acid (OCA), Compound 2 and Cenicriviroc (CVC), or Compound 2 and Elafibranor (ELA). Total (mg/liver) liver collagen 1 and 3 were determined by morphometry following Picro-Sirius Red staining. Data expressed as mean±SEM (n=12).

FIG. 5 total liver cholesterol following 8 weeks of treatment vehicle, Compound 2, Obeticholic Acid (OCA), Cenicriviroc (CVC), Elafibranor (ELA), Compound 2 and Obeticholic Acid (OCA), Compound 2 and Cenicriviroc (CVC), or Compound 2 and Elafibranor (ELA). Total (mg/liver) liver collagen 1 and 3 were determined by morphometry following Picro-Sirius Red staining. Data expressed as mean±SEM (n=12).

FIG. 6 total liver triglycerides following 8 weeks of treatment vehicle, Compound 2, Obeticholic Acid (OCA), Cenicriviroc (CVC), Elafibranor (ELA), Compound 2 and Obeticholic Acid (OCA), Compound 2 and Cenicriviroc (CVC), or Compound 2 and Elafibranor (ELA). Total (mg/liver) liver collagen 1 and 3 were determined by morphometry following Picro-Sirius Red staining. Data expressed as mean±SEM (n=12).

FIG. 7 is a summary of NALFD activity scores pre- and post-study biopsies following 8 weeks of treatment vehicle, Compound 2, Obeticholic Acid (OCA), Cenicriviroc (CVC), Elafibranor (ELA), Compound 2 and Obeticholic Acid (OCA), Compound 2 and Cenicriviroc (CVC), or Compound 2 and Elafibranor (ELA).

FIG. 8 shows change in NALFD activity scores from baseline based on pre- and post-study biopsies following 8 weeks of treatment with vehicle, Compound 2, Cenicriviroc (CVC), and Compound 2 and Cenicriviroc (CVC).

FIG. 9 shows mean percentage change in NALFD activity scores from baseline based on pre- and post-study biopsies following 8 weeks of treatment with vehicle, Compound 2, Obeticholic Acid (OCA), and Compound 2 and Obeticholic Acid (OCA).

FIG. 10 shows an overview of the results of fibrosis stage scores following 8 weeks of treatment vehicle, Compound 2, Obeticholic Acid (OCA), Cenicriviroc (CVC), Elafibranor (ELA), Compound 2 and Obeticholic Acid (OCA), Compound 2 and Cenicriviroc (CVC), or Compound 2 and Elafibranor (ELA). The change from pre- to post-study biopsy is indicated by a line. The points at each scoring step is slightly shifted to allow visual separation of the animals (this is done for visualization purposes and does not reflect any difference in score).

FIG. 11 shows mean percentage change in fibrosis stage from baseline based on pre- and post-study biopsies following 8 weeks of treatment with vehicle, Compound 2, Cenicriviroc (CVC), and Compound 2 and Cenicriviroc (CVC).

FIG. 12 shows mean percentage change in fibrosis stage from baseline based on pre- and post-study biopsies following 8 weeks of treatment with vehicle, Compound 2, Obeticholic Acid (OCA), and Compound 2 and Obeticholic Acid (OCA).

FIG. 13 shows mean percentage change in plasma total cholesterol levels from baseline based on pre- and post-study biopsies following 12 weeks of treatment with vehicle, Compound 2, Tropifexor, and Compound 2 and Tropifexor.

FIG. 14 shows mean percentage change in plasma total triglycerides from baseline based on pre- and post-study biopsies following 12 weeks of treatment with vehicle, Compound 2, Tropifexor, and Compound 2 and Tropifexor.

FIG. 15 shows mean percentage change in liver weight from baseline based on pre- and post-study biopsies following 12 weeks of treatment with vehicle, Compound 2, Tropifexor, and Compound 2 and Tropifexor.

FIG. 16 shows mean percentage change in liver total chloesterol from baseline based on pre- and post-study biopsies following 12 weeks of treatment with vehicle, Compound 2, Tropifexor, and Compound 2 and Tropifexor.

FIG. 17 shows mean percentage change in liver hydroxyproline from baseline based on pre- and post-study biopsies following 12 weeks of treatment with vehicle, Compound 2, Tropifexor, and Compound 2 and Tropifexor.

FIG. 18 shows an overview of the results of fibrosis stage scores following 12 weeks of treatment with vehicle, Compound 2, Tropifexor, and Compound 2 and Tropifexor. The change from pre- to post-study biopsy is indicated by a line. The points at each scoring step is slightly shifted to allow visual separation of the animals (this is done for visualization purposes and does not reflect any difference in score).

FIG. 19 shows mean percentage change in liver fibrosis based on pre- and post-study biopsies following 12 weeks of treatment with vehicle, Compound 2, Tropifexor, and Compound 2 and Tropifexor by morphometry following Picro-Sirius Red staining. Liver fibrosis was determined by morphometry following Picro-Sirius Red staining.

FIG. 20 is a summary of NALFD activity scores pre- and post-study biopsies following 12 weeks of treatment with vehicle, Compound 2, Tropifexor, and Compound 2 and Tropifexor.

FIG. 21 shows mean percentage change in steatosis scores from baseline based on pre- and post-study biopsies following 12 weeks of treatment with vehicle, Compound 2, Tropifexor, and Compound 2 and Tropifexor.

FIG. 22 shows the ratio of liver steatosis relative to vehicle following 12 weeks of treatment with vehicle, Compound 2, Tropifexor, and Compound 2 and Tropifexor.

FIG. 23 shows mean percentage change in liver collagen 1a1 following 12 weeks of treatment with vehicle, Compound 2, Tropifexor, and Compound 2 and Tropifexor.

FIG. 24 shows mean percentage change in liver α-SMA from baseline based on pre- and post-study biopsies following 12 weeks of treatment with vehicle, Compound 2, Tropifexor, and Compound 2 and Tropifexor.

FIG. 25 shows mean percentage change in liver galectin from baseline based on pre- and post-study biopsies following 12 weeks of treatment with vehicle, Compound 2, Tropifexor, and Compound 2 and Tropifexor.

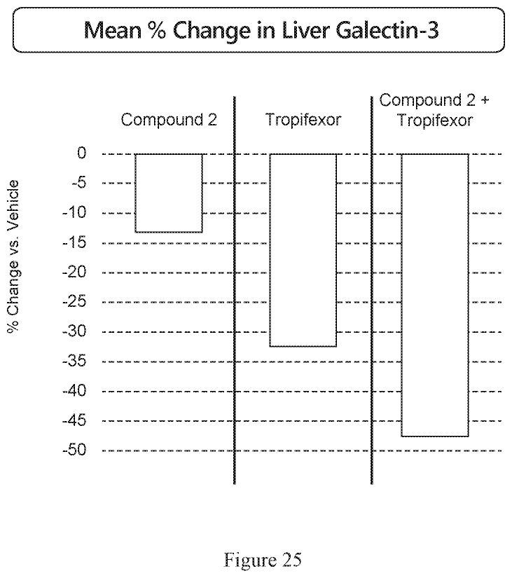

FIG. 26 shows plasma total cholesterol in mice following 12 weeks of treatment with vehicle, Compound 2, semaglutide, the combination of Compound 2 and semaglutide, tropifexor, and the combination of Compound 2 and tropifexor.

FIG. 27 shows total liver triglyceride content in mice following 12 weeks of treatment with vehicle, Compound 2, semaglutide, the combination of Compound 2 and semaglutide, tropifexor, and the combination of Compound 2 and tropifexor.

FIG. 28 shows total liver hydroxyproline content in mice following 12 weeks of treatment with vehicle, Compound 2, semaglutide, the combination of Compound 2 and semaglutide, tropifexor, and the combination of Compound 2 and tropifexor.

FIG. 29 shows total liver lipid content in mice following 12 weeks of treatment with vehicle, Compound 2, semaglutide, the combination of Compound 2 and semaglutide, tropifexor, and the combination of Compound 2 and tropifexor.

FIG. 30 shows mean percentage change in liver fibrosis based on pre- and post-study biopsies following 12 weeks of treatment with vehicle; Compound 2; semaglutide; the combination of Compound 2 and semaglutide; tropifexor; and the combination of Compound 2 and tropifexor.

FIG. 31 shows the ratio of liver triglycerides relative to vehicle following 12 weeks of treatment with vehicle, Compound 2, Tropifexor, and Compound 2 and Tropifexor.

FIG. 32 shows the change from baseline of NAFLD activity score following 12 weeks of treatment with vehicle, Compound 2, Tropifexor, and Compound 2 and Tropifexor.

FIG. 33 shows the ratio of liver steatosis relative to vehicle following 8 weeks of treatment with vehicle, Compound 2, Obeticholic Acid (OCA), and Compound 2 and Obeticholic Acid (OCA).

### DETAILED DESCRIPTION

Fatty acids consist of an alkyl chain with a terminal carboxyl group. Unsaturated fatty acids occur commonly in humans and contain up to six double bonds per chain. Most fatty acids in humans have a length of C16, C18 or C20. Fatty acids are stored primarily as esters of glycerol. Triglycerides (TGs) are triacylglycerols, i.e., where all three hydroxyls are esterified with a fatty acid, hi addition to TGs, glycerol esterified with only one fatty acid (monoacylglycerol) or two fatty acids (diacylgycerols, DAGs) are found. The distribution of esterification sites on glycerol is influenced by many factors and may have important biological function. Fatty acids are also used in the synthesis of other molecules, e.g., esters of cholesterol which can be degraded back to the parent molecule by esterases, and various phospholipids, including lysophosphatidic acid and phosphatide acid, which consist of phosphorylated acylated glycerols. Many of these products have biological activity suggesting that modulation of their levels may result in beneficial or detrimental effects.

Fatty acids are taken up by the liver from the circulation. Fatty acids derived from the diet enter the circulation after ingestion and passage through the lymphatic system. Once in the circulation the fatty acids are taken up by tissues and used as a source of energy either immediately or in the future. If not used immediately, the fatty acids are usually converted to TGs. Subsequently, TGs are hydrolyzed to generate the free fatty acids and glycerol. Both are often transported from cells such as adipocytes, which store large quantities of TGs, to the liver. Lipolysis of TGs occurs through the action of lipases. For example, lipoprotein lipase hydrolyzes triacylglycerols in plasma lipoproteins. Another example is hormone sensitive lipase (HSL), which hydrolyzes TGs stored in the adipocyte. HSL is very sensitive to certain hormones, such as insulin which inactivates the enzyme, glucagon, epinephrine, and ACTH.

Fatty acids in the liver are also supplied by de novo synthesis from small molecule intermediates derived from metabolic breakdown of sugars, amino acids and other fatty acids. Accordingly, excess dietary protein and carbohydrate are readily converted to fatty acids and stored as TGs. A key enzyme in fatty acid synthesis is acetyl-CoA carboxylase, which controls the overall synthesis of fatty acid by controlling the synthesis of malonyl CoA from acetyl CoA. Fatty acid synthase then catalyzes the addition of two carbon units to the activated carboxyl end of a growing chain. The result is the fatty acid palmitate. Palmitate is the precursor fatty acid for nearly all other fatty acids. Enzymes are available that lead to unsaturated fatty acids or elongated fatty acids.

Fatty acids are used for energy production primarily through oxidation in mitochondria. The first step entails conversion of the fatty acid to a fatty acyl CoA by acyl-CoA synthetase. Since the oxidizing enzymes are located inside the inner mitochondrial membrane and the membrane is impermeable to CoA and its derivatives, carnitine is used along with carnitine palmitoyltransferase (CPT) to transfer acyl-CoAs into the mitochondria. This step is rate-limiting in fatty acid oxidation. Two carbon units are removed from the carboxy terminus using four enzyme-catalyzed reactions. The product is acyl-CoA which can then be used in the synthesis of fatty acids (futile cycling), ketone bodies, or enters the TCA cycle where it is converted to CO₂ and ATP. Some of the energy generated by fatty acid oxidation is stored as ATP, some used in the biosynthesis of other molecules, while some is lost in the form of heat. Agents that increase heat production can enable net energy expenditure.

Fat accumulation occurs when there is net energy intake relative to energy expenditure. Energy is often stored as fat, more specifically TGs. Ideally, fat is stored in the adipocyte which is its natural storage site. When in excess, however, fat is stored in other tissues, some of which can be negatively affected. Fat accumulation in the liver will depend on a multitude of factors, including fatty acid delivery from the circulation, lipogenesis (i.e., de novo lipid synthesis) in the liver, and free fatty acid oxidation.

Nonalcoholic fatty liver disease (NAFLD) is a clinicopathological term that encompasses a disease spectrum ranging from simple TG accumulation in hepatocytes to hepatic steatosis with inflammation (nonalcoholic steatohepatitis, NASH) to fibrosis and cirrhosis. NAFLD is the most frequent cause of liver enzyme elevations. The prevalence of NAFLD in the population is estimated to be 14-28%. Hepatic insulin resistance is associated with hepatic steatosis.

Products from TG metabolism, e.g., DAGs and long chain AcylCoAs (LCACoA) are thought to negatively effect insulin response through effects on the insulin receptor phosphorylation. Long chain CoAs and DAG increase Ser/Thr phosphorylation of insulin receptor substrates (IRS 1-3) and thereby disrupt Tyr phosphorylation of these substrates by the insulin receptor. The resulting hepatic insulin resistance contributes to the development of compensatory hyperinsulinemia which further drives fat accumulation via SREBP1. Reduction in TGs may reduce the levels of DAGs and LCACoAs and therefore improve the response to insulin. Improved response to insulin may also diminish further fat accumulation.

Oxidative stress results from an imbalance between pro-oxidant and antioxidant chemical species that leads to oxidative damage. Oxidation of fatty acids is an important source of reactive oxygen species (ROS). Some of the consequences of increased ROS is depleted ATP, destruction of membranes via lipid peroxidation, and release of proinflammatory cytokines. An increase in liver triglycerides may lead to increased oxidative stress in the hepatocytes, and the progression of hepatic steatosis to NASH. Human livers with NASH have increased lipid peroxidation and impaired mitochondrial function. This can result in cell death, hepatic stellate cell activation and fibrosis and inflammation. All of these activities may cause patients with NAFLD to be at risk for NASH, a more serious disease with higher risk of liver cirrhosis and hepatocellular carcinoma. TH is known to increase fatty acid oxidation and mitochondrial enzyme activity which could result in increased ROS and liver damage. Prodrugs that are activated by P450s may also cause an increase in ROS.

The present disclosure relates to the use of TR-β agonists in combination with one or more second pharmaceutical agents, in methods of decreasing fat content in the liver of an animal comprising administering to said animal a therapeutically effective amount of a TR-β agonist compound, a pharmaceutically acceptable salt thereof, or prodrugs thereof or pharmaceutically acceptable salts of said prodrugs, and one or more second pharmaceutical agents. The disclosure further relates to methods of preventing, treating, or ameliorating fatty liver disease in an animal comprising administering to said animal a therapeutically effective amount of a TR-β agonist compound, a pharmaceutically acceptable salt thereof, or prodrugs thereof or pharmaceutically acceptable salts of said prodrugs, and one or more second pharmaceutical agents. The compounds of Formula I and/or the second pharmaceutical agent may be an active form or a prodrug thereof. Further included in the present disclosure is the use of pharmaceutically acceptable salts, including but not limited to acid addition salts and physiological salts, and co-crystals of said compounds of Formula I and/or the second pharmaceutical agents. Further included in the present disclosure is the use of prodrugs of compounds of Formula I and/or the second pharmaceutical agents that are active forms, and pharmaceutically acceptable salts, including but not limited to acid addition salts and physiological salts, and co-crystals thereof.

### Definitions

The term “mammal” is used in its usual biological sense. Thus, it specifically includes humans and non-human mammals such as dogs, cats, horses, donkeys, mules, cows, domestic buffaloes, camels, llamas, alpacas, bison, yaks, goats, sheep, pigs, elk, deer, domestic antelopes, and non-human primates as well as many other species.

“Subject” as used herein, means a human or a non-human mammal including but not limited to a dog, cat, horse, donkey, mule, cow, domestic buffalo, camel, llama, alpaca, bison, yak, goat, sheep, pig, elk, deer, domestic antelope, or a non-human primate selected for treatment or therapy.

“Subject in need thereof” means a subject identified as in need of a therapy or treatment.

A therapeutic effect relieves, to some extent, one or more of the symptoms of a disease or disorder, and includes curing the disease or disorder. “Curing” means that the symptoms of active disease are eliminated. However, certain long-term or permanent effects of the disease may exist even after a cure is obtained (such as extensive tissue damage).

The phrase “therapeutically effective amount” means an amount of a compound or a combination of compounds that ameliorates, attenuates or eliminates one or more of the symptoms of a particular disease or condition or prevents, modifies, or delays the onset of one or more of the symptoms of a particular disease or condition.

“Treat,” “treatment,” or “treating,” as used herein refers to administering a pharmaceutical composition for prophylactic and/or therapeutic purposes. The term “prophylactic treatment” refers to treating a patient who does not yet have the relevant disease or disorder, but who is susceptible to, or otherwise at risk of, a particular disease or disorder, whereby the treatment reduces the likelihood that the patient will develop the disease or disorder. The term “therapeutic treatment” refers to administering treatment to a patient already having a disease or disorder.

“Preventing” or “prevention” refers to delaying or forestalling the onset, development or progression of a condition or disease for a period of time, including weeks, months, or years.

“Amelioration” means a lessening of severity of at least one indicator of a condition or disease. In certain embodiments, amelioration includes a delay or slowing in the progression of one or more indicators of a condition or disease. The severity of indicators may be determined by subjective or objective measures which are known to those skilled in the art.

“Modulation” means a perturbation of function or activity. In certain embodiments, modulation means an increase in gene expression. In certain embodiments, modulation means a decrease in gene expression. In certain embodiments, modulation means an increase or decrease in total serum levels of a specific protein. In certain embodiments, modulation means an increase or decrease in free serum levels of a specific protein. In certain embodiments, modulation means an increase or decrease in total serum levels of a specific non-protein factor. In certain embodiments, modulation means an increase or decrease in free serum levels of a specific non-protein factor. In certain embodiments, modulation means an increase or decrease in total bioavailability of a specific protein. In certain embodiments, modulation means an increase or decrease in total bioavailability of a specific non-protein factor.

“Administering” means providing a pharmaceutical agent or composition to a subject, and includes, but is not limited to, administering by a medical professional and self-administering.

Administration of the compounds disclosed herein or the pharmaceutically acceptable salts thereof, or the second pharmaceutical agents disclosed herein can be via any of the accepted modes of administration for agents that serve similar utilities including, but not limited to, orally, subcutaneously, intravenously, intranasally, topically, transdermally, intraperitoneally, intramuscularly, intrapulmonarilly, vaginally, rectally, or intraocularly. Oral and parenteral administrations are customary in treating the indications that are the subject of the preferred embodiments.

“Parenteral administration,” means administration through injection or infusion. Parenteral administration includes, but is not limited to, subcutaneous administration, intravenous administration, intramuscular administration, intraarterial administration, and intracranial administration.

“Subcutaneous administration” means administration just below the skin.

“Intravenous administration” means administration into a vein.

“Intraarterial administration” means administration into an artery.

The term “agent” includes any substance, molecule, element, compound, entity, or a combination thereof. It includes, but is not limited to, e.g., protein, polypeptide, peptide or mimetic, small organic molecule, polysaccharide, polynucleotide, and the like. It can be a natural product, a synthetic compound, or a chemical compound, or a combination of two or more substances.

“Pharmaceutical agent” means a substance that provides a therapeutic effect when administered to a subject.

“Pharmaceutical composition” means a mixture of substances suitable for administering to an individual that includes a pharmaceutical agent. For example, a pharmaceutical composition may comprise a modified oligonucleotide and a sterile aqueous solution.

“Active pharmaceutical ingredient” means the substance in a pharmaceutical composition that provides a desired effect.

The term “pharmaceutically acceptable salt” refers to salts that retain the biological effectiveness and properties of the compounds with which they are associated and, which are not biologically or otherwise undesirable. In many cases, the compounds herein are capable of forming acid and/or base salts by virtue of the presence of phenol and/or phosphonate groups or groups similar thereto. One of ordinary skill in the art will be aware that the protonation state of any or all of these compounds may vary with pH and ionic character of the surrounding solution, and thus the present disclosure contemplates multiple charge states of each compound. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297, Johnston et al., published Sep. 11, 1987 (incorporated by reference herein in its entirety).

“Solvate” refers to the compound formed by the interaction of a solvent and an EPI, a metabolite, or salt thereof. Suitable solvates are pharmaceutically acceptable solvates including hydrates.

The term “prodrug” as used herein refers to any compound that when administered to a biological system generates a biologically active compound as a result of spontaneous chemical reaction(s), enzyme catalyzed chemical reaction(s), and/or metabolic chemical reaction(s), or a combination of each. Standard prodrugs are formed using groups attached to functionality, e.g., HO—, HS—, HOOC—, R₂N—, associated with the drug, that cleave in vivo. Standard prodrugs include but are not limited to carboxylate esters where the group is alkyl, aryl, aralkyl, acyloxyalkyl, alkoxycarbonyloxyalkyl as well as esters of hydroxyl, thiol and amines where the group attached is an acyl group, an alkoxycarbonyl, aminocarbonyl, phosphate or sulfate. The groups illustrated are exemplary, not exhaustive, and one skilled in the art could prepare other known varieties of prodrugs. Such prodrugs of the compounds of the present disclosure fall within this scope. Prodrugs must undergo some form of a chemical transformation to produce the compound that is biologically active or is a precursor of the biologically active compound. In some cases, the prodrug is biologically active, usually less than the drug itself, and serves to improve drug efficacy or safety through improved oral bioavailability, and/or pharmacodynamic half-life, etc. Prodrug forms of compounds may be utilized, for example, to improve bioavailability, improve subject acceptability such as by masking or reducing unpleasant characteristics such as bitter taste or gastrointestinal irritability, alter solubility such as for intravenous use, provide for prolonged or sustained release or delivery, improve ease of formulation, or provide site-specific delivery of the compound. Prodrugs are described in The Organic Chemistry of Drug Design and Drug Action, by Richard B. Silverman, Academic Press, San Diego, 1992. Chapter 8: “Prodrugs and Drug delivery Systems” pp. 352-401; Design of Prodrugs, edited by H. Bundgaard, Elsevier Science, Amsterdam, 1985; Design of Biopharmaceutical Properties through Prodrugs and Analogs, Ed. by E. B. Roche, American Pharmaceutical Association, Washington, 1977; and Drug Delivery Systems, ed. by R. L. Juliano, Oxford Univ. Press, Oxford, 1980.

T groups that have more than one atom are read from left to right wherein the left atom of the T group is connected to the phenyl group bearing the R¹ and R² groups, and the right atom of the T group is linked to the carbon, phosphorus, or other atom in X or E. For example, when T is —O—CH₂— or —N(H)C(O)— it means -phenyl-O—CH₂—X and -phenyl-N(H)C(O)—X.

The term “alkyl” refers to a straight or branched or cyclic chain hydrocarbon radical with only single carbon-carbon bonds. Representative examples include methyl, ethyl, propyl, isopropyl, cyclopropyl, butyl, isobutyl, tert-butyl, cyclobutyl, pentyl, cyclopentyl, hexyl, and cyclohexyl, all of which may be optionally substituted. Alkyl groups are C₁-C₂₀.

The term “aryl” refers to aromatic groups which have 5-14 ring atoms and at least one ring having a conjugated pi electron system and includes carbocyclic aryl, heterocyclic aryl and biaryl groups, all of which may be optionally substituted.

Carbocyclic aryl groups are groups which have 6-14 ring atoms wherein the ring atoms on the aromatic ring are carbon atoms. Carbocyclic aryl groups include monocyclic carbocyclic aryl groups and polycyclic or fused compounds such as optionally substituted naphthyl groups.

Heterocyclic aryl or heteroaryl groups are groups which have 5-14 ring atoms wherein 1 to 4 heteroatoms are ring atoms in the aromatic ring and the remainder of the ring atoms being carbon atoms. Suitable heteroatoms include oxygen, sulfur, nitrogen, and selenium. Suitable heteroaryl groups include furanyl, thienyl, pyridyl, pyrrolyl, N-lower alkyl pyrrolyl, pyridyl-N-oxide, pyrimidyl, pyrazinyl, imidazolyl, and the like, all optionally substituted.

The term “biaryl” represents aryl groups which have 5-14 atoms containing more than one aromatic ring including both fused ring systems and aryl groups substituted with other aryl groups. Such groups may be optionally substituted. Suitable biaryl groups include naphthyl and biphenyl.

The term “optionally substituted” or “substituted” includes groups substituted by one, two, three, four, five, or six substituents, independently selected from lower alkyl, lower aryl, lower aralkyl, lower cyclic alkyl, lower heterocycloalkyl, hydroxy, lower alkoxy, lower aryloxy, perhaloalkoxy, aralkoxy, lower heteroaryl, lower heteroaryloxy, lower heteroarylalkyl, lower heteroaralkoxy, azido, amino, halo, lower alkylthio, oxo, lower acylalkyl, lower carboxy esters, carboxyl, -carboxamido, nitro, lower acyloxy, lower aminoalkyl, lower alkylaminoaryl, lower alkylaryl, lower alkylaminoalkyl, lower alkoxyaryl, lower arylamino, lower aralkylamino, sulfonyl, lower-carboxamidoalkylaryl, lower-carboxamidoaryl, lower hydroxyalkyl, lower haloalkyl, lower alkylaminoalkylcarboxy-, lower aminocarboxamidoalkyl-, cyano, lower alkoxyalkyl, lower perhaloalkyl, and lower arylalkyloxyalkyl.

“Substituted aryl” and “substituted heteroaryl” refers to aryl and heteroaryl groups substituted with 1-3 substituents. These substituents are selected from the group consisting of lower alkyl, lower alkoxy, lower perhaloalkyl, halo, hydroxy, and amino.

The term “-aralkyl” refers to an alkylene group substituted with an aryl group. Suitable aralkyl groups include benzyl, picolyl, and the like, and may be optionally substituted. “Heteroarylalkyl” refers to an alkylene group substituted with a heteroaryl group.

The term “alkylaryl-” refers to an aryl group substituted with an alkyl group. “Lower alkylaryl-” refers to such groups where alkyl is lower alkyl.

The term “lower” referred to herein in connection with organic radicals or compounds respectively refers to 6 carbon atoms or less. Such groups may be straight chain, branched, or cyclic.

The term “higher” referred to herein in connection with organic radicals or compounds respectively refers to 7 or more carbon atoms. Such groups may be straight chain, branched, or cyclic.

The term “cyclic alkyl” or “cycloalkyl” refers to alkyl groups that are cyclic of 3 to 10 carbon atoms, and in one aspect are 3 to 6 carbon atoms Suitable cyclic groups include norbornyl and cyclopropyl. Such groups may be substituted.

The term “heterocyclic,” “heterocyclic alkyl” or “heterocycloalkyl” refer to cyclic groups of 3 to 10 atoms, and in one aspect are 3 to 6 atoms, containing at least one heteroatom, in a further aspect are 1 to 3 heteroatoms. Suitable heteroatoms include oxygen, sulfur, and nitrogen. Heterocyclic groups may be attached through a nitrogen or through a carbon atom in the ring. The heterocyclic alkyl groups include unsaturated cyclic, fused cyclic and spirocyclic groups. Suitable heterocyclic groups include pyrrolidinyl, morpholino, morpholinoethyl, and pyridyl.

The terms “arylamino” (a), and “aralkylamino” (b), respectively, refer to the group —NRR′ wherein respectively, (a) R is aryl and R′ is hydrogen, alkyl, aralkyl, heterocycloalkyl, or aryl, and (b) R′ is aralkyl and R′ is hydrogen, aralkyl, aryl, alkyl or heterocycloalkyl.

The term “acyl” refers to —C(O)R where R is alkyl, heterocycloalkyl, or aryl.

The term “carboxy esters” refers to —C(O)OR where R is alkyl, aryl, aralkyl, cyclic alkyl, or heterocycloalkyl, all optionally substituted.

The term “carboxyl” refers to —C(O)OH.

The term “oxo” refers to ═O in an alkyl or heterocycloalkyl group.

The term “amino” refers to —NRR′ where R and R′ are independently selected from hydrogen, alkyl, aryl, aralkyl and heterocycloalkyl, all except H are optionally substituted; and R and R′ can form a cyclic ring system.

The term “-carboxylamido” refers to —CONR₂ where each R is independently hydrogen or alkyl.

The term “-sulphonylamido” or “-sulfonylamido” refers to —S(═O)₂NR₂ where each R is independently hydrogen or alkyl.

The term “halogen” or “halo” refers to —F, —Cl, —Br and —I.

The term “alkylaminoalkylcarboxy” refers to the group alkyl-NR-alk-C(O)—O— where “alk” is an alkylene group, and R is a H or lower alkyl.

The term “sulphonyl” or “sulfonyl” refers to —SO₂R, where R is H, alkyl, aryl, aralkyl, or heterocycloalkyl.

The term “sulphonate” or “sulfonate” refers to —SO₂OR, where R is —H, alkyl, aryl, aralkyl, or heterocycloalkyl.

The term “alkenyl” refers to unsaturated groups which have 2 to 12 atoms and contain at least one carbon-carbon double bond and includes straight-chain, branched-chain and cyclic groups. Alkenyl groups may be optionally substituted. Suitable alkenyl groups include allyl. “1-Alkenyl” refers to alkenyl groups where the double bond is between the first and second carbon atom. If the 1-alkenyl group is attached to another group, e.g., it is a W substituent attached to the cyclic phosphonate, it is attached at the first carbon.

The term “alkynyl” refers to unsaturated groups which have 2 to 12 atoms and contain at least one carbon-carbon triple bond and includes straight-chain, branched-chain and cyclic groups. Alkynyl groups may be optionally substituted. Suitable alkynyl groups include ethynyl. “1-Alkynyl” refers to alkynyl groups where the triple bond is between the first and second carbon atom. If the 1-alkynyl group is attached to another group, e.g., it is a W substituent attached to the cyclic phosphonate, it is attached at the first carbon.

The term “alkylene” refers to a divalent straight chain, branched chain or cyclic saturated aliphatic group. In one aspect the alkylene group contains up to and including 10 atoms. In another aspect the alkylene group contains up to and including 6 atoms. In a further aspect the alkylene group contains up to and including 4 atoms. The alkylene group can be either straight, branched or cyclic.

The term “acyloxy” refers to the ester group —O—C(O)R, where R is H, alkyl, alkenyl, alkynyl, aryl, aralkyl, or heterocycloalkyl.

The term “aminoalkyl-” refers to the group NR₂-alk- wherein “alk” is an alkylene group and R is selected from —H, alkyl, aryl, aralkyl, and heterocycloalkyl.

The term “alkylaminoalkyl-” refers to the group alkyl-NR-alk- wherein each “alk” is an independently selected alkylene, and R is H or lower alkyl. “Lower alkylaminoalkyl-” refers to groups where the alkyl and the alkylene group is lower alkyl and alkylene, respectively.

The term “arylaminoalkyl-” refers to the group aryl-NR-alk- wherein “alk” is an alkylene group and R is —H, alkyl, aryl, aralkyl, or heterocycloalkyl. In “lower arylaminoalkyl-,” the alkylene group is lower alkylene.

The term “alkylaminoaryl-” refers to the group alkyl-NR-aryl- wherein “aryl” is a divalent group and R is —H, alkyl, aralkyl, or heterocycloalkyl. In “lower alkylaminoaryl-,” the alkyl group is lower alkyl.

The term “alkoxyaryl-” refers to an aryl group substituted with an alkyloxy group. In “lower alkyloxyaryl-,” the alkyl group is lower alkyl.

The term “aryloxyalkyl-” refers to an alkyl group substituted with an aryloxy group.

The term “aralkyloxyalkyl-” refers to the group aryl-alk-O-alk- wherein “alk” is an alkylene group. “Lower aralkyloxyalkyl-” refers to such groups where the alkylene groups are lower alkylene.

The term “alkoxy-” or “alkyloxy-” refers to the group alkyl-O—.

The term “alkoxyalkyl-” or “alkyloxyalkyl-” refer to the group alkyl-O-alk- wherein “alk” is an alkylene group. In “lower alkoxyalkyl-,” each alkyl and alkylene is lower alkyl and alkylene, respectively.

The term “alkylthio-” refers to the group alkyl-S—.

The term “alkylthioalkyl-” refers to the group alkyl-5-alk- wherein “alk” is an alkylene group. In “lower alkylthioalkyl-,” each alkyl and alkylene is lower alkyl and alkylene, respectively.

The term “alkoxycarbonyloxy-” refers to alkyl-O—C(O)—O—.

The term “aryloxycarbonyloxy-” refers to aryl-O—C(O)—O—.

The term “alkylthiocarbonyloxy-” refers to alkyl-S—C(O)—O—.

The term “amido” refers to the NR₂ group next to an acyl or sulfonyl group as in NR₂—C(O)—, RC(O)—NR¹—, NR₂—S(═O)₂— and RS(═O)₂—NR¹—, where R and R¹ include —H, alkyl, aryl, aralkyl, and heterocycloalkyl.

The term “carboxamido” refer to NR₂—C(O)— and RC(O)—NR¹—, where R and R¹ include —H, alkyl, aryl, aralkyl, and heterocycloalkyl. The tern does not include urea, —NR—C(O)—NR—.

The terms “sulphonamido” or “sulfonamido” refer to NR₂—S(═O)₂— and RS(═O)₂—NR¹—, where R and R¹ include —H, alkyl, aryl, aralkyl, and heterocycloalkyl. The term does not include sulfonylurea, —NR—S(═O)₂—NR—.

The term “carboxamidoalkylaryl” and “carboxamidoaryl” refers to an aryl-alk-NR¹—C(O), and ar-NR¹—C(O)-alk-, respectively where “ar” is aryl, “alk” is alkylene, R¹ and R include H, alkyl, aryl, aralkyl, and heterocycloalkyl.

The term “sulfonamidoalkylaryl” and “sulfonamidoaryl” refers to an aryl-alk-NR¹—S(═O)₂—, and ar-NR¹—S(═O)₂—, respectively where “ar” is aryl, “alk” is alkylene, R¹ and R include —H, alkyl, aryl, aralkyl, and heterocycloalkyl.

The term “hydroxyalkyl” refers to an alkyl group substituted with one —OH.

The term “haloalkyl” refers to an alkyl group substituted with halo.

The term “cyano” refers to —C≡N.

The term “nitro” refers to —NO₂.

The term “acylalkyl” refers to an alkyl-C(O)-alk-, where “alk” is alkylene.

The term “aminocarboxamidoalkyl-” refers to the group NR₂—C(O)—N(R)-alk- wherein R is an alkyl group or H and “alk” is an alkylene group. “Lower aminocarboxamidoalkyl-” refers to such groups wherein “alk” is lower alkylene.

The term “heteroarylalkyl” refers to an alkylene group substituted with a heteroaryl group.

The term “perhalo” refers to groups wherein every C—H bond has been replaced with a C-halo bond on an aliphatic or aryl group. Suitable perhaloalkyl groups include —CF₃ and —CFCl₂.

### Compounds

In some embodiments, the compounds for use as described herein include compounds according to Formula I:

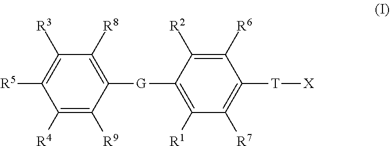

wherein:

G is selected from the group consisting of —O—, —S—, —S(═O)—, —S(═O)₂—, —Se—, —CH₂—, —CF₂—, —CHF—, —C(O)—, —CH(OH)—, —CH(C₁-C₄ alkyl)-, —CH(C₁-C₄ alkoxy)-, —C(═CH₂)—, —NH—, and —N(C₁-C₄ alkyl)-;

T is selected from the group consisting of —(CR^{a}₂)ₖ—, —CR^{b}═CR^{b}—(CR^{a}₂)ₙ—, —(CR^{a}₂)ₙ—CR^{b}═CR^{b}—, —(CR^{a}₂)—CR^{b}═CR^{b}—(CR^{a}₂)—, —O(CR^{b}₂)(CR^{a}₂)ₙ—, —S(CR^{b}₂)(CR^{a}₂)ₙ—, N(R^{c})(CR^{b}₂)(CR^{a}₂)ₙ—, N(R^{b})C(O)(CR^{a}₂)ₙ, —C(O)(CR^{a}₂)ₘ—, —(CR^{a}₂)ₘC(O)—, —(CR^{a}₂)C(O)(CR^{a}₂)ₙ, —(CR^{a}₂)ₙC(O)(CR^{a}₂)—, and —C(O)NH(CR^{b}₂)(CR^{a}₂)_{P}—;

k is an integer from 1-4;

m is an integer from 0-3;

n is an integer from 0-2;

p is an integer from 0-1;

each R^{a} is independently selected from the group consisting of hydrogen, optionally substituted —C₁-C₄ alkyl, halogen, —OH, optionally substituted —O—C₁-C₄ alkyl, —OCF₃, optionally substituted —S—C₁-C₄ alkyl, —NR^{b}R^{c}, optionally substituted —C₂-C₄ alkenyl, and optionally substituted —C₂-C₄ alkynyl; with the proviso that when one R^{a} is attached to C through an O, S, or N atom, then the other R^{a} attached to the same C is a hydrogen, or attached via a carbon atom;

each R^{b} is independently selected from the group consisting of hydrogen and optionally substituted —C₁-C₄ alkyl;

each R^{c} is independently selected from the group consisting of hydrogen and optionally substituted —C₁-C₄ alkyl, optionally substituted —C(O)—C₁-C₄ alkyl, and —C(O)H;

R¹, and R² are each independently selected from the group consisting of halogen, optionally substituted —C₁-C₄ alkyl, optionally substituted —S—C₁-C₃ alkyl, optionally substituted —C₂-C₄ alkenyl, optionally substituted —C₂-C₄ alkynyl, —CF₃, —OCF₃, optionally substituted-O—C₁-C₃ alkyl, and cyano;

R⁶, R⁷, R⁸, and R⁹ are each independently selected from the group consisting of are each independently selected from the group consisting of hydrogen, halogen, optionally substituted —C C₁-C₄ alkyl, optionally substituted —S—C₁-C₃ alkyl, optionally substituted —C₂-C₄ alkenyl, optionally substituted —C₂-C₄ alkynyl, —CF₃, —OCF₃, optionally substituted-O—C₁-C₃ alkyl, and cyano; or R⁶ and T are taken together along with the carbons they are attached to form a ring of 5 to 6 atoms including 0 to 2 heteroatoms independently selected from —NRⁱ—, —O—, and —S—, with the proviso that when there are 2 heteroatoms in the ring and both heteroatoms are different than nitrogen then both heteroatoms have to be separated by at least one carbon atom; and X is attached to this ring by a direct bond to a ring carbon, or by —(CR^{a}₂)— or —C(O)— bonded to a ring carbon or a ring nitrogen;

Rⁱ is selected from the group consisting of hydrogen, —C(O)C₁-C₄ alkyl, —C₁-C₄ alkyl, and —C₁-C₄-aryl;

R³ and R⁴ are independently selected from the group consisting of hydrogen, halogen, —CF₃, —OCF₃, cyano, optionally substituted —C₁-C₁₂ alkyl, optionally substituted —C₂-C₁₂ alkenyl, optionally substituted —C₂-C₁₂ alkynyl, —SR^{d}, —S(═O)R^{e}, —S(═O)₂R^{e}, —S(═O)₂NR^{f}R^{g}, —C(O)OR^{h}, —C(O)R^{e}, —N(R^{b})C(O)NR^{f}R^{g}, —N(R^{b})S(═O)₂R^{e}, —N(R^{b})S(═O)₂NR^{f}R^{g}, and —NR^{f}R^{g};

each R^{d} is selected from the group consisting of optionally substituted —C₁-C₁₂ alkyl, optionally substituted —C₂-C₁₂ alkenyl, optionally substituted —C₂-C₁₂ alkynyl, optionally substituted —(CR^{b}₂)ₙ aryl, optionally substituted —(CR^{b}₂)ₙ cycloalkyl, optionally substituted —(CR^{b}₂)₁₁ heterocycloalkyl, and —C(O)NR^{f}R^{g};

each R^{e} is selected from the group consisting of optionally substituted —C₁-C₁₂ alkyl, optionally substituted —C₂-C₁₂ alkenyl, optionally substituted —C₂-C₁₂ alkynyl, optionally substituted —(CR^{a}₂)ₙ aryl, optionally substituted —(CR^{a}₂)ₙ cycloalkyl, and optionally substituted —(CR^{a}₂)ₙ heterocycloalkyl;

R^{f} and R^{g} are each independently selected from the group consisting of hydrogen, optionally substituted —C₁-C₁₂ alkyl, optionally substituted —C₂-C₁₂ alkenyl, optionally substituted —C₂-C₁₂ alkynyl, optionally substituted —(CR^{b}₂)ₙ aryl, optionally substituted —(CR^{b}₂)₁₁ cycloalkyl, and optionally substituted —(CR^{b}₂)ₙ heterocycloalkyl, or R^{f} and R^{g} may together form an optionally substituted heterocyclic ring, which may contain a second heterogroup selected from the group consisting of O, NR^{C}, and S, wherein said optionally substituted heterocyclic ring may be substituted with 0-4 substituents selected from the group consisting of optionally substituted —C₁-C₄ alkyl, —OR^{b}, oxo, cyano, —CF₃, optionally substituted phenyl, and —C(O)OR^{h};

each R^{h} is selected from the group consisting of optionally substituted —C₁-C₁₂ alkyl, optionally substituted —C₂-C₁₂ alkenyl, optionally substituted —C₂-C₁₂ alkynyl, optionally substituted —(CR^{b}₂)ₙ aryl, optionally substituted —(CR^{b}₂)ₙ cycloalkyl, and optionally substituted —(CR^{b}₂)ₙ heterocycloalkyl;

R⁵ is selected from the group consisting of —OH, optionally substituted —OC₁-C₆ alkyl, OC(O)R^{e}, —OC(O)OR^{h}, —F, —NHC(O)R^{e}, —NHS(═O)R^{e}, —NHS(═O)₂R^{e}, —NHC(═S)NH(R^{h}), and —NHC(O)NH(R^{h});

X is P(O)YR¹¹Y′R¹¹;

Y and Y′ are each independently selected from the group consisting of —O—, and —NR^{v}—; when Y and Y′ are —O—, R¹¹ attached to —O— is independently selected from the group consisting of —H, alkyl, optionally substituted aryl, optionally substituted heterocycloalkyl, optionally substituted CH₂-heterocycloakyl wherein the cyclic moiety contains a carbonate or thiocarbonate, optionally substituted -alkylaryl, —C(R^{z})₂OC(O)NR^{z}₂, —NR^{z}—C(O)—R^{y}, —C(R^{z})₂—OC(O)R^{y}, —C(R^{z})₂—O—C(O)OR^{y}, —C(R^{z})₂OC(O)SR^{y}, -alkyl-S—C(O)R^{y}, -alkyl-S—S-alkylhydroxy, and -alkyl-S—S—S-alkylhydroxy;

when Y and Y′ are —NR^{v}—, then R¹¹ attached to —NR^{v}— is independently selected from the group consisting of —H, —[C(R^{z})₂]q-COOR^{y}, —C(R^{x})₂COOR^{Y}, —[C(R^{z})₂]q-C(O)SR^{y}, and -cycloalkylene-COOR^{y};

when Y is —O— and Y′ is NR^{v}, then R¹¹ attached to —O— is independently selected from the group consisting of —H, alkyl, optionally substituted aryl, optionally substituted heterocycloalkyl, optionally substituted CH₂-heterocycloakyl wherein the cyclic moiety contains a carbonate or thiocarbonate, optionally substituted -alkylaryl, —C(R^{z})₂OC(O)NR^{z}₂, —NR^{z}—C(O)—R^{y}, —C(R^{z})₂—OC(O)R^{y}, —C(R^{z})₂—O—C(O)OR^{y}, —C(R^{z})₂OC(O)SR^{y},

-alkyl-S—C(O)R^{y}, -alkyl-S—S-alkylhydroxy, and -alkyl-S—S—S-alkylhydroxy; and R¹¹ attached to —NR^{v}— is independently selected from the group consisting of H, —[C(R^{z})₂]_{q}—COOR^{y}, —C(R^{x})₂COOR^{y}, —[C(R^{z})₂]_{q}—C(O)SR^{y}, and -cycloalkylene-COOR^{y};

or when Y and Y′ are independently selected from —O— and NR^{v}, then together R¹¹ and R¹¹ are -alkyl-S—S-alkyl- to form a cyclic group, or together R¹¹ and R¹¹ are the group:

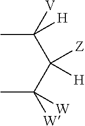

wherein:

V, W, and W′ are independently selected from the group consisting of hydrogen, optionally substituted alkyl, optionally substituted aralkyl, heterocycloalkyl, aryl, substituted aryl, heteroaryl, substituted heteroaryl, optionally substituted 1-alkenyl, and optionally substituted 1-alkynyl;

or together V and Z are connected via an additional 3-5 atoms to form a cyclic group containing 5-7 atoms, wherein 0-1 atoms are heteroatoms and the remaining atoms are carbon, substituted with hydroxy, acyloxy, alkylthiocarbonyloxy, alkoxycarbonyloxy, or aryloxycarbonyloxy attached to a carbon atom that is three atoms from both Y groups attached to the phosphorus;

or together V and Z are connected via an additional 3-5 atoms to form a cyclic group, wherein 0-1 atoms are heteroatoms and the remaining atoms are carbon, that is fused to an aryl group at the beta and gamma position to the Y attached to the phosphorus;

or together V and W are connected via an additional 3 carbon atoms to form an optionally substituted cyclic group containing 6 carbon atoms and substituted with one substituent selected from the group consisting of hydroxy, acyloxy, alkoxycarbonyloxy, alkylthiocarbonyloxy, and aryloxycarbonyloxy, attached to one of said carbon atoms that is three atoms from a Y attached to the phosphorus;

or together Z and W are connected via an additional 3-5 atoms to form a cyclic group, wherein 0-1 atoms are heteroatoms and the remaining atoms are carbon, and V must be aryl, substituted aryl, heteroaryl, or substituted heteroaryl;

or together W and W′ are connected via an additional 2-5 atoms to form a cyclic group, wherein 0-2 atoms are heteroatoms and the remaining atoms are carbon, and V must be aryl, substituted aryl, heteroaryl, or substituted heteroaryl;

Z is selected from the group consisting of —CHR^{z}OH, —CHR^{z}OC(O)R^{y}, —CHR^{z}OC(S)R^{y}, —CHR^{z}OC(S)OR^{y}, —CHR^{z}OC(O)SR^{y}, —CHR^{z}OCO₂R^{y}, —OR^{z}, —SR^{z}, —CHR^{z}N₃, —CH₂-aryl, —CH(aryl)OH, —CH(CH═CR^{z}₂)OH, —CH(C≡CR^{z})OH, —R^{z}, —NR^{z}₂, —OCOR^{y}, —OCO₂R^{y}, —SCOR^{y}, —SCO₂R^{y}, —NHCOR^{z}, —NHCO₂R^{y}, —CH₂NH-aryl, —(CH₂)q-OR^{z}, and —(CH₂)q-SR^{z};

q is an integer 2 or 3;

each R^{z} is selected from the group consisting of R^{y} and —H;

each R^{y} is selected from the group consisting of alkyl, aryl, heterocycloalkyl, and aralkyl;

each R^{x} is independently selected from the group consisting of —H, and alkyl, or together R^{x} and R^{x} form a cyclic alkyl group;

each R^{v} is selected from the group consisting of —H, lower alkyl, acyloxyalkyl, alkoxycarbonyloxyalkyl, and lower acyl;

and pharmaceutically acceptable salts thereof.

In some embodiments, the compound of Formula I has the following provisos:

a) when G is —O—, T is —CH₂—, R¹ and R² are each bromo, R³ is iso-propyl, R⁴ is hydrogen, and R⁵ is —OH, then X is not P(O)(OH)₂ or P(O)(OCH₂CH₃)₂;

b) V, Z, W, W′ are not all —H; and

c) when Z is —R^{z}, then at least one of V, W, and W′ is not —H, alkyl, aralkyl, or heterocycloalkyl;

d) when G is —O—, T is —(CH₂)₁₋₄—, R¹ and R² are independently halogen, alkyl, and cycloalkyl, R³ is alkyl, R⁴ is hydrogen, and R⁵ is —OH, then X is not —P(O)(OH)₂ or —P(O)(O-lower alkyl)₂; and

e) when G is —O—, R⁵ is —NHC(O)R^{e}, —NHS(═O)₁₋₂R^{e}, —NHC(S)NH(R^{b}), or —NHC(O)NH(R^{h}), T is —(CH₂)^{m}—, —CH═CH—, —O(CH₂)₁₋₂—, or —NH(CH₂)₁₋₂—, then X is not —P(O)(OH)₂ or —P(O)(OH)NH₂.

In some embodiments, the compound is selected from one or more of the following:

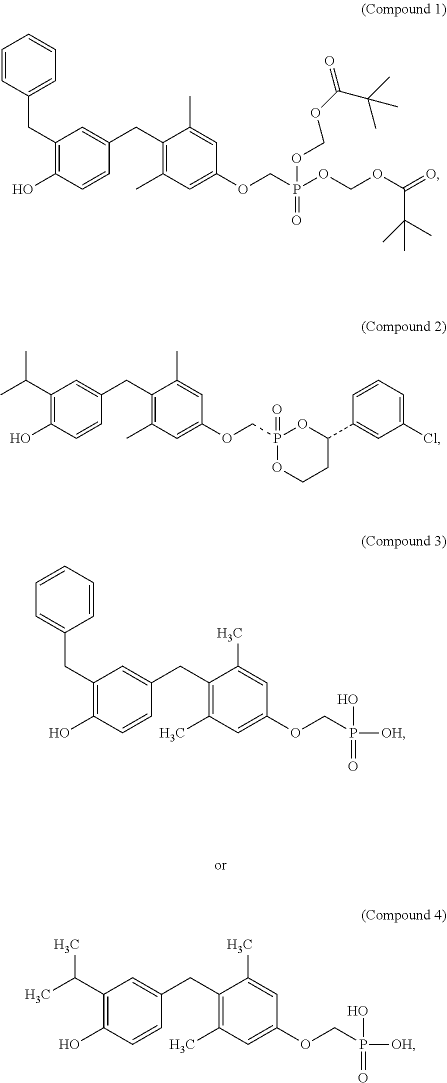

or pharmaceutically acceptable salts thereof.

In other embodiments, the compound is selected from:

| Structure | Compound Number |
|---|---|
| 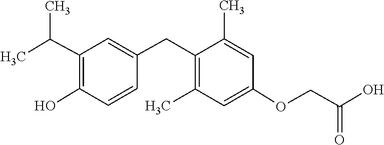 [SMILES: Cc1cc(OCC(=O)O)cc(C)c1Cc1ccc(O)c(C(C)C)c1] | 17 |
| 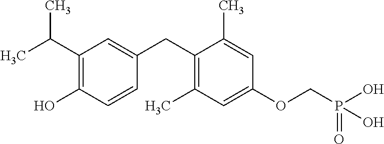 [SMILES: Cc1cc(OCP(=O)(O)O)cc(C)c1Cc1ccc(O)c(C(C)C)c1] | 7 |
| 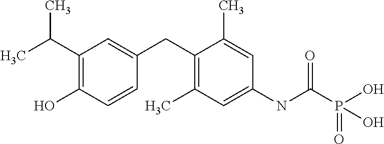 [SMILES: Cc1cc(NC(=O)P(=O)(O)O)cc(C)c1Cc1ccc(O)c(C(C)C)c1] | 1a |
| 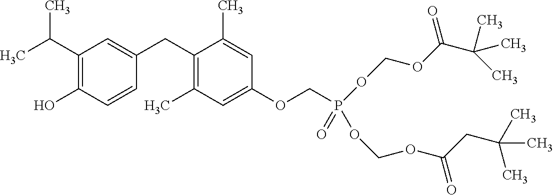 [SMILES: Cc1cc(OCP(=O)(OCOC(=O)CC(C)(C)C)OCOC(=O)C(C)(C)C)cc(C)c1Cc1ccc(O)c(C(C)C)c1] | 12-1 |
| 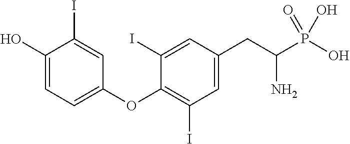 [SMILES: NC(Cc1cc(I)c(Oc2ccc(O)c(I)c2)c(I)c1)P(=O)(O)O] | 2a |
| 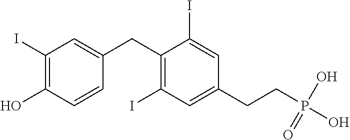 [SMILES: O=P(O)(O)CCc1cc(I)c(Cc2ccc(O)c(I)c2)c(I)c1] | 3a |
| 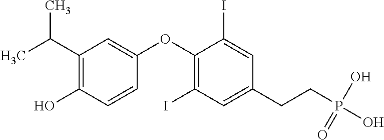 [SMILES: CC(C)c1cc(Oc2c(I)cc(CCP(=O)(O)O)cc2I)ccc1O] | 4a |
| 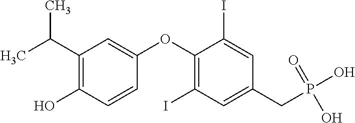 [SMILES: CC(C)c1cc(Oc2c(I)cc(CP(=O)(O)O)cc2I)ccc1O] | 5 |
| 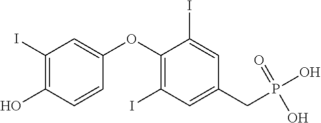 [SMILES: O=P(O)(O)Cc1cc(I)c(Oc2ccc(O)c(I)c2)c(I)c1] | 6 |
| 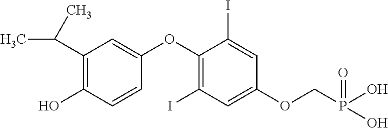 [SMILES: CC(C)c1cc(Oc2c(I)cc(OCP(=O)(O)O)cc2I)ccc1O] | 8 |
| 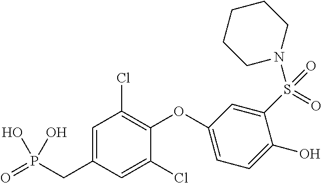 [SMILES: O=P(O)(O)Cc1cc(Cl)c(Oc2ccc(O)c(S(=O)(=O)N3CCCCC3)c2)c(Cl)c1] | 9 |
| 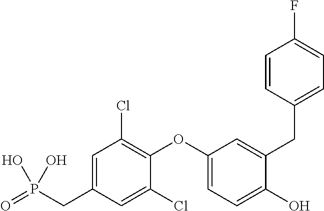 [SMILES: O=P(O)(O)Cc1cc(Cl)c(Oc2ccc(O)c(Cc3ccc(F)cc3)c2)c(Cl)c1] | 11 |
| 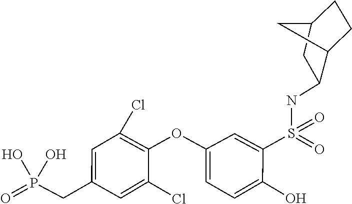 | 10 |
| 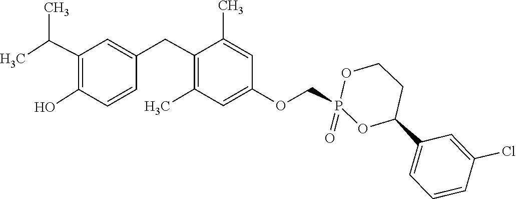 [SMILES: Cc1cc(OC[P@@]2(=O)OCC[C@@H](c3cccc(Cl)c3)O2)cc(C)c1Cc1ccc(O)c(C(C)C)c1] | cis-13-1 |
| 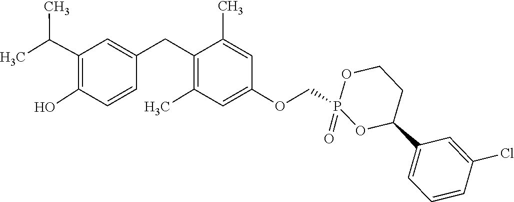 [SMILES: Cc1cc(OC[P@]2(=O)OCC[C@@H](c3cccc(Cl)c3)O2)cc(C)c1Cc1ccc(O)c(C(C)C)c1] | trans-13-1 |
| 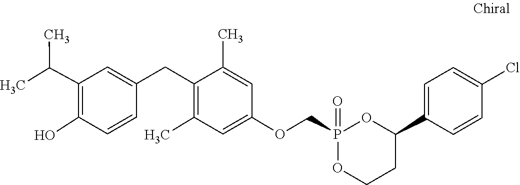 [SMILES: Cc1cc(OC[P@]2(=O)OCC[C@H](c3ccc(Cl)cc3)O2)cc(C)c1Cc1ccc(O)c(C(C)C)c1] | cis-13-6 |
| 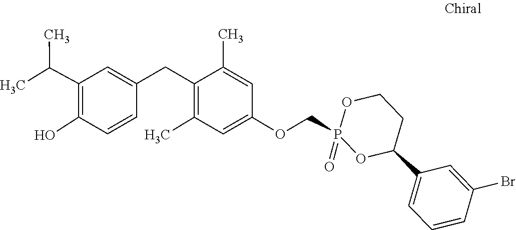 [SMILES: Cc1cc(OC[P@@]2(=O)OCC[C@@H](c3cccc(Br)c3)O2)cc(C)c1Cc1ccc(O)c(C(C)C)c1] | cis-13-2 |
| 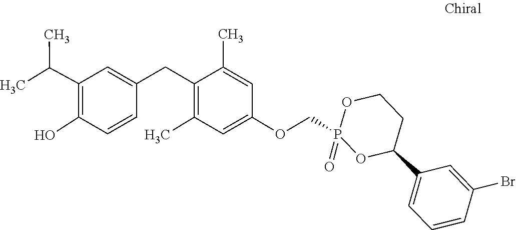 [SMILES: Cc1cc(OC[P@]2(=O)OCC[C@@H](c3cccc(Br)c3)O2)cc(C)c1Cc1ccc(O)c(C(C)C)c1] | trans-13-2 |
| 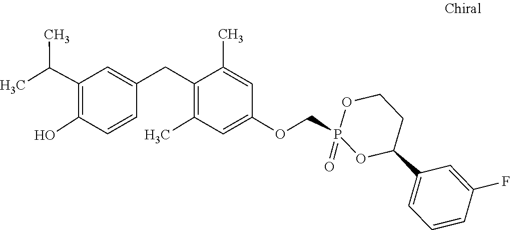 [SMILES: Cc1cc(OC[P@@]2(=O)OCC[C@@H](c3cccc(F)c3)O2)cc(C)c1Cc1ccc(O)c(C(C)C)c1] | cis-13-3 |
| 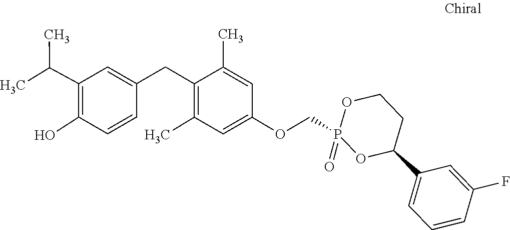 [SMILES: Cc1cc(OC[P@]2(=O)OCC[C@@H](c3cccc(F)c3)O2)cc(C)c1Cc1ccc(O)c(C(C)C)c1] | trans-13-3 |
| 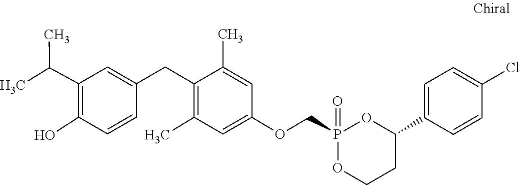 [SMILES: Cc1cc(OC[P@]2(=O)OCC[C@@H](c3ccc(Cl)cc3)O2)cc(C)c1Cc1ccc(O)c(C(C)C)c1] | trans-13-6 |
| 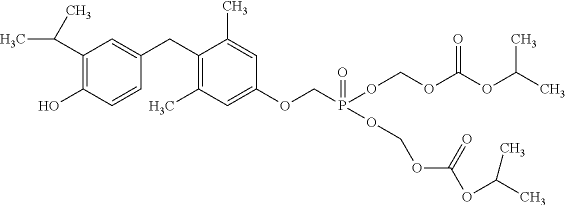 [SMILES: Cc1cc(OCP(=O)(OCOC(=O)OC(C)C)OCOC(=O)OC(C)C)cc(C)c1Cc1ccc(O)c(C(C)C)c1] | 12-3 |
| 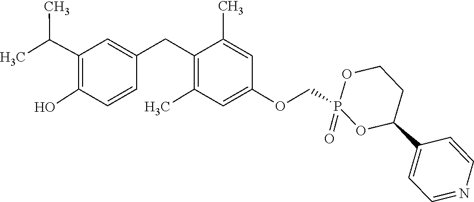 [SMILES: Cc1cc(OC[P@]2(=O)OCC[C@@H](c3ccncc3)O2)cc(C)c1Cc1ccc(O)c(C(C)C)c1] | trans-13-5 |
| 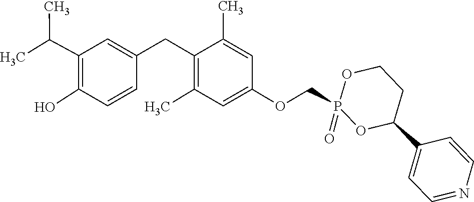 [SMILES: Cc1cc(OC[P@@]2(=O)OCC[C@@H](c3ccncc3)O2)cc(C)c1Cc1ccc(O)c(C(C)C)c1] | cis-13-5 |
| 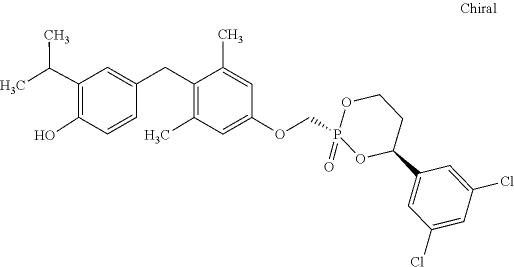 [SMILES: Cc1cc(OC[P@]2(=O)OCC[C@@H](c3cc(Cl)cc(Cl)c3)O2)cc(C)c1Cc1ccc(O)c(C(C)C)c1] | trans-13-7 |
| 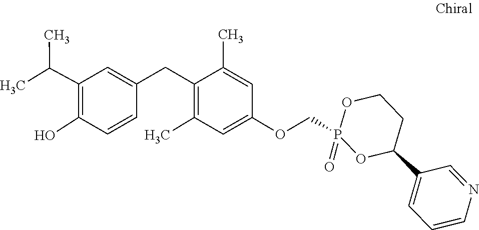 [SMILES: Cc1cc(OC[P@]2(=O)OCC[C@@H](c3cccnc3)O2)cc(C)c1Cc1ccc(O)c(C(C)C)c1] | trans-13-4 |
| 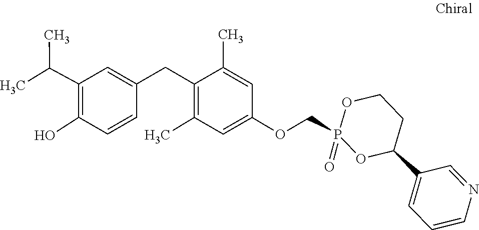 [SMILES: Cc1cc(OC[P@@]2(=O)OCC[C@@H](c3cccnc3)O2)cc(C)c1Cc1ccc(O)c(C(C)C)c1] | cis-13-4 |
| 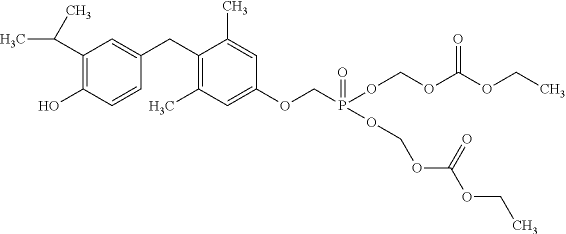 [SMILES: CCOC(=O)OCOP(=O)(COc1cc(C)c(Cc2ccc(O)c(C(C)C)c2)c(C)c1)OCOC(=O)OCC] | 12-2 |
| 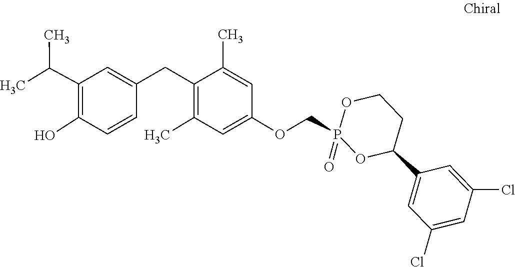 [SMILES: Cc1cc(OC[P@@]2(=O)OCC[C@@H](c3cc(Cl)cc(Cl)c3)O2)cc(C)c1Cc1ccc(O)c(C(C)C)c1] | cis-13-7 |
| 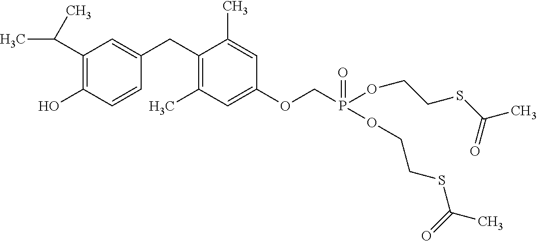 [SMILES: CC(=O)SCCOP(=O)(COc1cc(C)c(Cc2ccc(O)c(C(C)C)c2)c(C)c1)OCCSC(C)=O] | 14 |
| 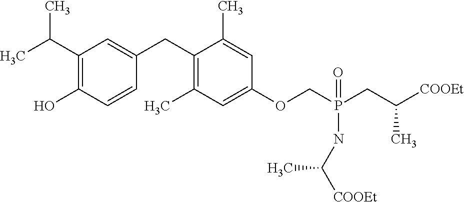 [SMILES: CCOC(=O)[C@H](C)CP(=O)(COc1cc(C)c(Cc2ccc(O)c(C(C)C)c2)c(C)c1)N[C@@H](C)C(=O)OCC] | 15-1 |
| 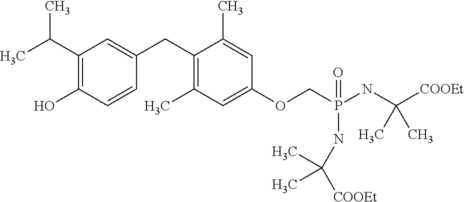 [SMILES: CCOC(=O)C(C)(C)NP(=O)(COc1cc(C)c(Cc2ccc(O)c(C(C)C)c2)c(C)c1)NC(C)(C)C(=O)OCC] | 15-2 |
| 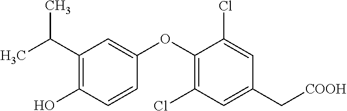 [SMILES: CC(C)c1cc(Oc2c(Cl)cc(CC(=O)O)cc2Cl)ccc1O] | 18 |
| 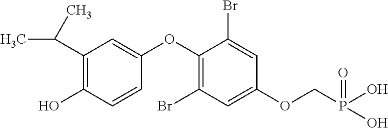 [SMILES: CC(C)c1cc(Oc2c(Br)cc(OCP(=O)(O)O)cc2Br)ccc1O] | 8-1 |
| 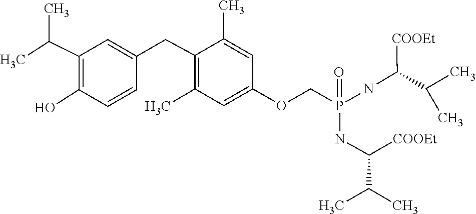 | 15-3 |
| 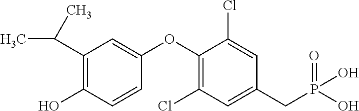 [SMILES: CC(C)c1cc(Oc2c(Cl)cc(CP(=O)(O)O)cc2Cl)ccc1O] | 19 |
| 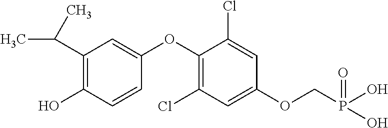 [SMILES: CC(C)c1cc(Oc2c(Cl)cc(OCP(=O)(O)O)cc2Cl)ccc1O] | 8-2 |
| 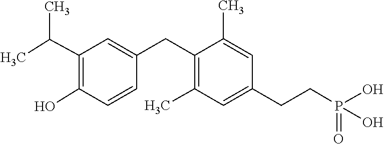 [SMILES: Cc1cc(CCP(=O)(O)O)cc(C)c1Cc1ccc(O)c(C(C)C)c1] | 24-1 |
| 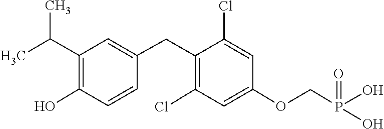 [SMILES: CC(C)c1cc(Cc2c(Cl)cc(OCP(=O)(O)O)cc2Cl)ccc1O] | 7-5 |
| 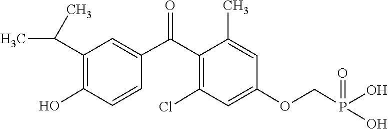 [SMILES: Cc1cc(OCP(=O)(O)O)cc(Cl)c1C(=O)c1ccc(O)c(C(C)C)c1] | 25 |
| 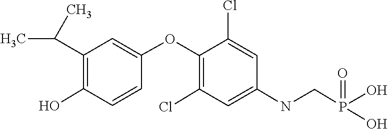 [SMILES: CC(C)c1cc(Oc2c(Cl)cc(NCP(=O)(O)O)cc2Cl)ccc1O] | 22 |
| 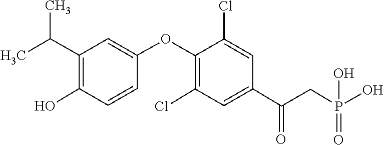 [SMILES: CC(C)c1cc(Oc2c(Cl)cc(C(=O)CP(=O)(O)O)cc2Cl)ccc1O] | 21 |
| 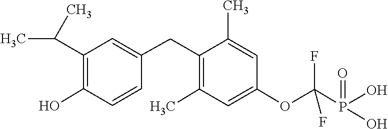 [SMILES: Cc1cc(OC(F)(F)P(=O)(O)O)cc(C)c1Cc1ccc(O)c(C(C)C)c1] | 7-6 |
| 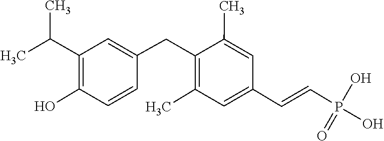 [SMILES: Cc1cc(/C=C/P(=O)(O)O)cc(C)c1Cc1ccc(O)c(C(C)C)c1] | 24-2 |
| 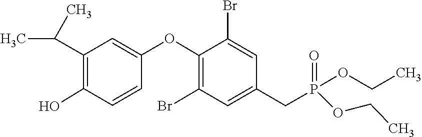 [SMILES: CCOP(=O)(Cc1cc(Br)c(Oc2ccc(O)c(C(C)C)c2)c(Br)c1)OCC] | 19-1 |
| 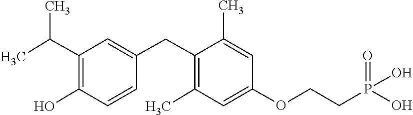 [SMILES: Cc1cc(OCCP(=O)(O)O)cc(C)c1Cc1ccc(O)c(C(C)C)c1] | 26 |
| 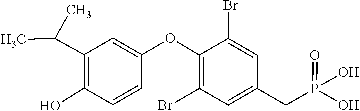 [SMILES: CC(C)c1cc(Oc2c(Br)cc(CP(=O)(O)O)cc2Br)ccc1O] | 19-2 |
| 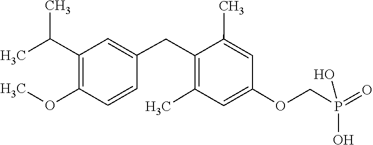 [SMILES: COc1ccc(Cc2c(C)cc(OCP(=O)(O)O)cc2C)cc1C(C)C] | 7-4 |
| 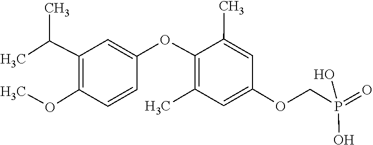 [SMILES: COc1ccc(Oc2c(C)cc(OCP(=O)(O)O)cc2C)cc1C(C)C] | 30 |
| 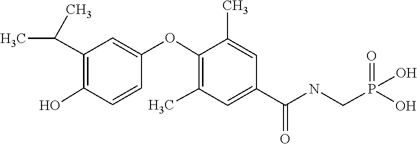 | 23 |
| 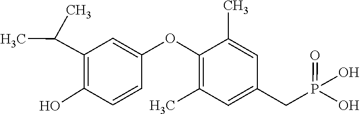 [SMILES: Cc1cc(CP(=O)(O)O)cc(C)c1Oc1ccc(O)c(C(C)C)c1] | 19-3 |
| 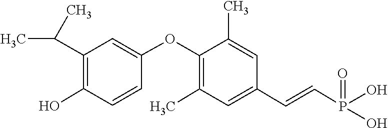 [SMILES: Cc1cc(/C=C/P(=O)(O)O)cc(C)c1Oc1ccc(O)c(C(C)C)c1] | 28 |
| 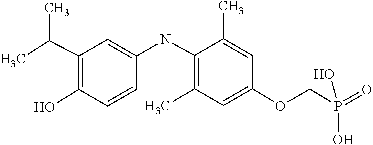 [SMILES: Cc1cc(OCP(=O)(O)O)cc(C)c1Nc1ccc(O)c(C(C)C)c1] | 20 |
| 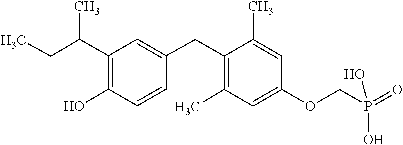 [SMILES: CCC(C)c1cc(Cc2c(C)cc(OCP(=O)(O)O)cc2C)ccc1O] | 7-3 |
| 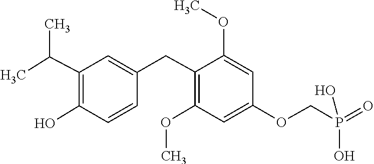 [SMILES: COc1cc(OCP(=O)(O)O)cc(OC)c1Cc1ccc(O)c(C(C)C)c1] | 7-2 |
| 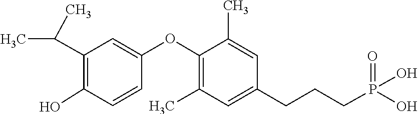 [SMILES: Cc1cc(CCCP(=O)(O)O)cc(C)c1Oc1ccc(O)c(C(C)C)c1] | 29 |
| 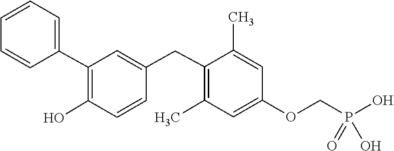 [SMILES: Cc1cc(OCP(=O)(O)O)cc(C)c1Cc1ccc(O)c(-c2ccccc2)c1] | 7-1 |
| 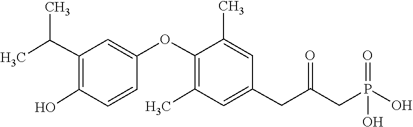 [SMILES: Cc1cc(CC(=O)CP(=O)(O)O)cc(C)c1Oc1ccc(O)c(C(C)C)c1] | 32 |
| 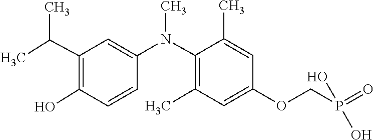 [SMILES: Cc1cc(OCP(=O)(O)O)cc(C)c1N(C)c1ccc(O)c(C(C)C)c1] | 20-1 |
| 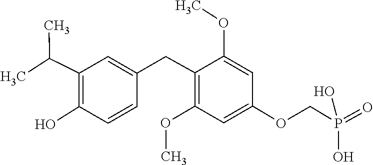 [SMILES: COc1cc(OCP(=O)(O)O)cc(OC)c1Cc1ccc(O)c(C(C)C)c1] | 24 |
| 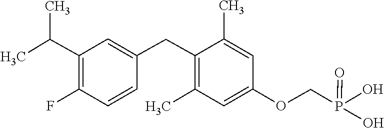 [SMILES: Cc1cc(OCP(=O)(O)O)cc(C)c1Cc1ccc(F)c(C(C)C)c1] | 27 |
| 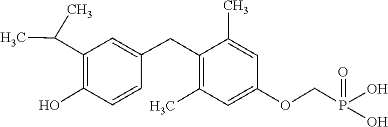 [SMILES: Cc1cc(OCP(=O)(O)O)cc(C)c1Cc1ccc(O)c(C(C)C)c1] | 31 |
| 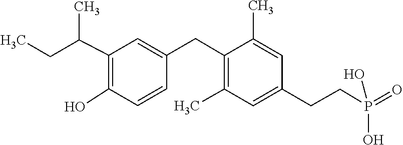 [SMILES: CCC(C)c1cc(Cc2c(C)cc(CCP(=O)(O)O)cc2C)ccc1O] | 24-3 |
| 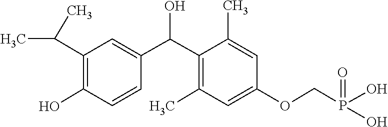 [SMILES: Cc1cc(OCP(=O)(O)O)cc(C)c1C(O)c1ccc(O)c(C(C)C)c1] | 33 |
| 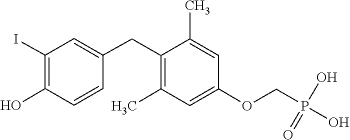 [SMILES: Cc1cc(OCP(=O)(O)O)cc(C)c1Cc1ccc(O)c(I)c1] | 34 |
| 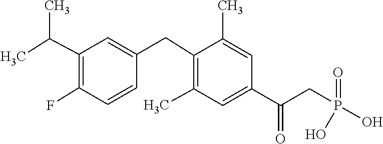 [SMILES: Cc1cc(C(=O)CP(=O)(O)O)cc(C)c1Cc1ccc(F)c(C(C)C)c1] | 41-2 |
| 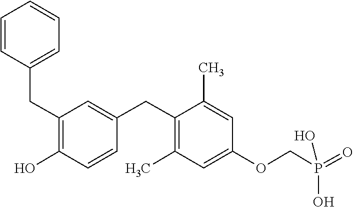 [SMILES: Cc1cc(OCP(=O)(O)O)cc(C)c1Cc1ccc(O)c(Cc2ccccc2)c1] | 38 |
| 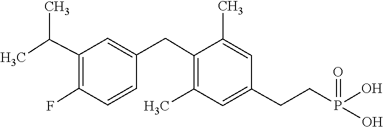 [SMILES: Cc1cc(CCP(=O)(O)O)cc(C)c1Cc1ccc(F)c(C(C)C)c1] | 42-2 |
| 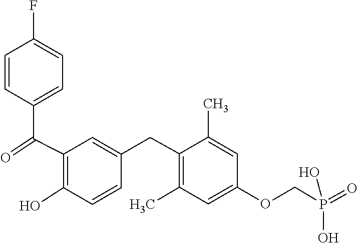 [SMILES: Cc1cc(OCP(=O)(O)O)cc(C)c1Cc1ccc(O)c(C(=O)c2ccc(F)cc2)c1] | 39 |
| 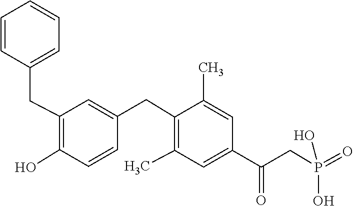 [SMILES: Cc1cc(C(=O)CP(=O)(O)O)cc(C)c1Cc1ccc(O)c(Cc2ccccc2)c1] | 41 |
| 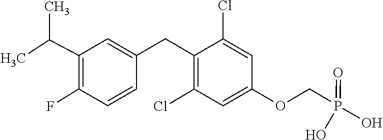 [SMILES: CC(C)c1cc(Cc2c(Cl)cc(OCP(=O)(O)O)cc2Cl)ccc1F] | 27-2 |
| 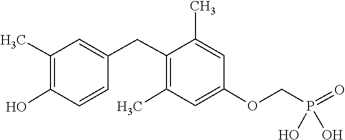 [SMILES: Cc1cc(Cc2c(C)cc(OCP(=O)(O)O)cc2C)ccc1O] | 7-7 |
| 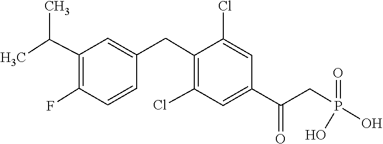 [SMILES: CC(C)c1cc(Cc2c(Cl)cc(C(=O)CP(=O)(O)O)cc2Cl)ccc1F] | 41-3 |
| 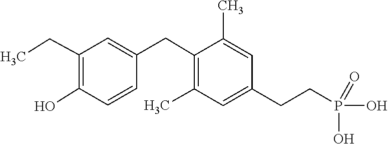 [SMILES: CCc1cc(Cc2c(C)cc(CCP(=O)(O)O)cc2C)ccc1O] | 24-4 |
| 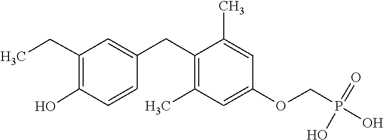 [SMILES: CCc1cc(Cc2c(C)cc(OCP(=O)(O)O)cc2C)ccc1O] | 7-8 |
| 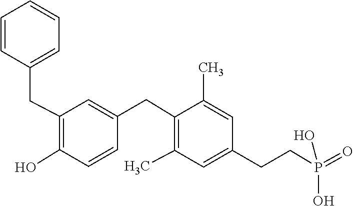 [SMILES: Cc1cc(CCP(=O)(O)O)cc(C)c1Cc1ccc(O)c(Cc2ccccc2)c1] | 42 |
| 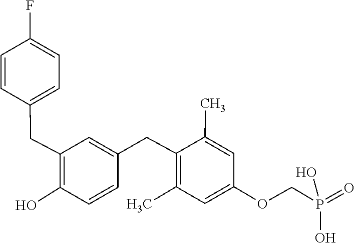 [SMILES: Cc1cc(OCP(=O)(O)O)cc(C)c1Cc1ccc(O)c(Cc2ccc(F)cc2)c1] | 40 |
| 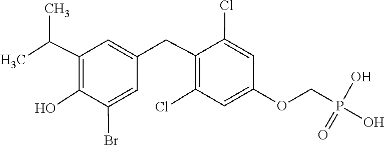 [SMILES: CC(C)c1cc(Cc2c(Cl)cc(OCP(=O)(O)O)cc2Cl)cc(Br)c1O] | 7-14 |
| 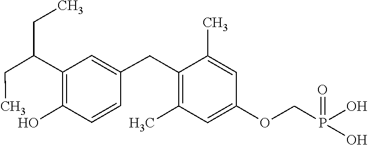 [SMILES: CCC(CC)c1cc(Cc2c(C)cc(OCP(=O)(O)O)cc2C)ccc1O] | 7-9 |
| 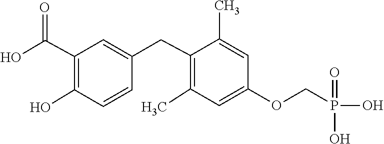 [SMILES: Cc1cc(OCP(=O)(O)O)cc(C)c1Cc1ccc(O)c(C(=O)O)c1] | 35 |
| 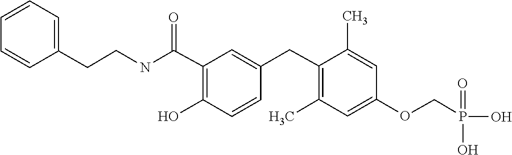 | 37 |
| 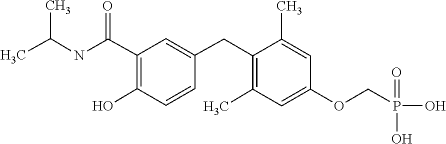 | 36 |
| 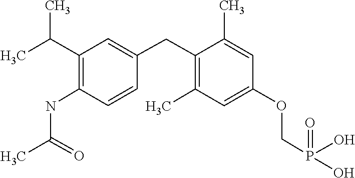 | 7-12 |
| 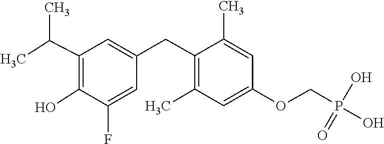 [SMILES: Cc1cc(OCP(=O)(O)O)cc(C)c1Cc1cc(F)c(O)c(C(C)C)c1] | 7-11 |
| 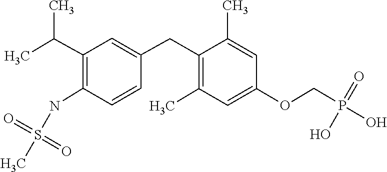 | 7-13 |
| 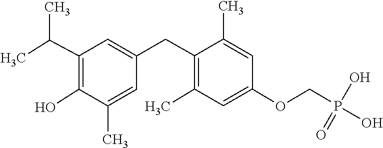 [SMILES: Cc1cc(Cc2c(C)cc(OCP(=O)(O)O)cc2C)cc(C(C)C)c1O] | 7-10 |
| 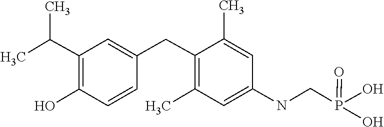 | 47 |
| 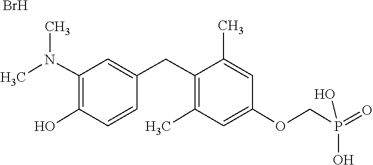 [SMILES: Br ; Cc1cc(OCP(=O)(O)O)cc(C)c1Cc1ccc(O)c(N(C)C)c1] | 49 |
| 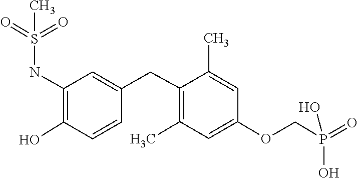 | 51-1 |
| 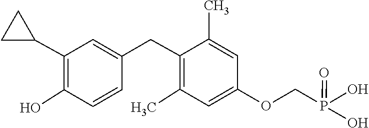 [SMILES: Cc1cc(OCP(=O)(O)O)cc(C)c1Cc1ccc(O)c(C2CC2)c1] | 48 |
| 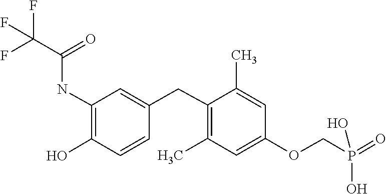 | 51-2 |
| 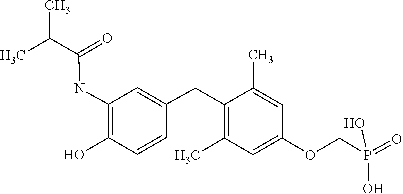 | 51-3 |
| 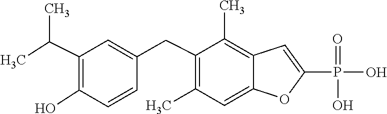 [SMILES: Cc1cc2oc(P(=O)(O)O)cc2c(C)c1Cc1ccc(O)c(C(C)C)c1] | 45 |
| 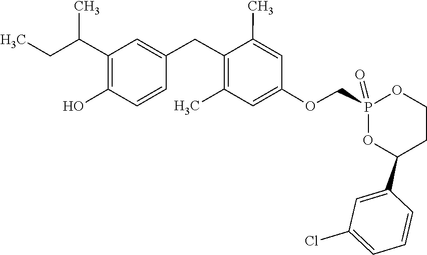 [SMILES: CCC(C)c1cc(Cc2c(C)cc(OC[P@@]3(=O)OCC[C@@H](c4cccc(Cl)c4)O3)cc2C)ccc1O] | 13-8 |
| 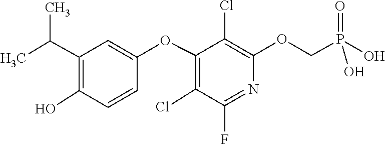 [SMILES: CC(C)c1cc(Oc2c(Cl)c(F)nc(OCP(=O)(O)O)c2Cl)ccc1O] | 57 |
| 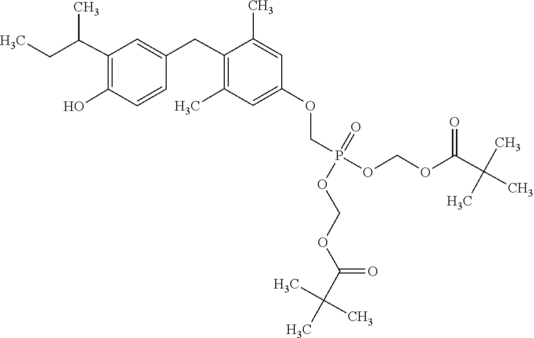 [SMILES: CCC(C)c1cc(Cc2c(C)cc(OCP(=O)(OCOC(=O)C(C)(C)C)OCOC(=O)C(C)(C)C)cc2C)ccc1O] | 12-4 |
| 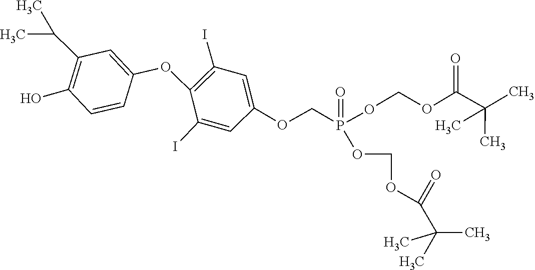 [SMILES: CC(C)c1cc(Oc2c(I)cc(OCP(=O)(OCOC(=O)C(C)(C)C)OCOC(=O)C(C)(C)C)cc2I)ccc1O] | 12-7 |
| 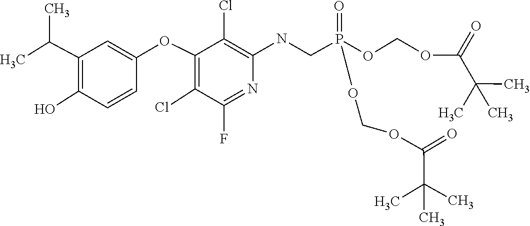 | 12-9 |
| 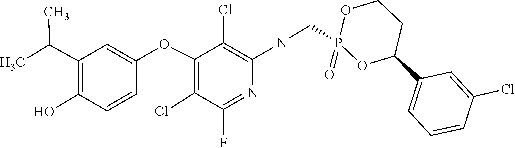 | 13-12-trans |
| 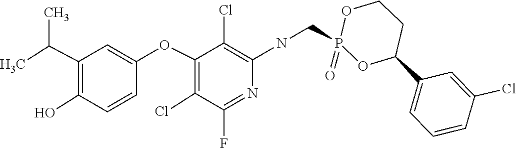 | 13-12-cis |
| 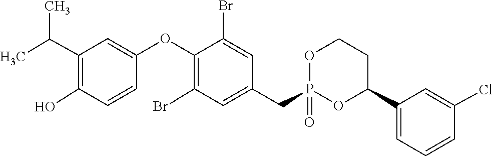 [SMILES: CC(C)c1cc(Oc2c(Br)cc(C[P@@]3(=O)OCC[C@@H](c4cccc(Cl)c4)O3)cc2Br)ccc1O] | 13-9 |
| 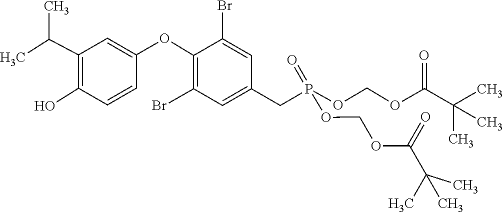 [SMILES: CC(C)c1cc(Oc2c(Br)cc(CP(=O)(OCOC(=O)C(C)(C)C)OCOC(=O)C(C)(C)C)cc2Br)ccc1O] | 12-5 |
| 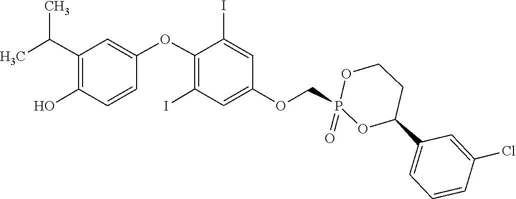 [SMILES: CC(C)c1cc(Oc2c(I)cc(OC[P@@]3(=O)OCC[C@@H](c4cccc(Cl)c4)O3)cc2I)ccc1O] | 13-10 |
| 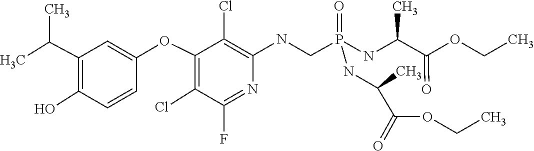 | 15-6 |
| 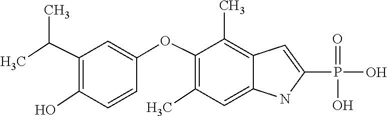 | 66 |
| 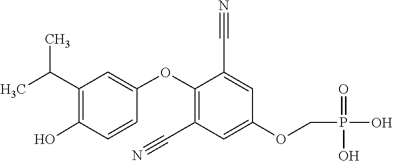 [SMILES: CC(C)c1cc(Oc2c(C#N)cc(OCP(=O)(O)O)cc2C#N)ccc1O] | 56 |
| 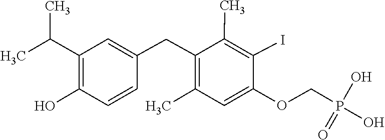 [SMILES: Cc1cc(OCP(=O)(O)O)c(I)c(C)c1Cc1ccc(O)c(C(C)C)c1] | 46 |
| 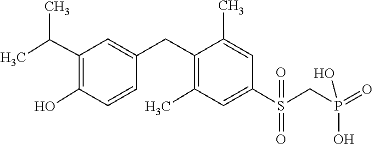 [SMILES: Cc1cc(S(=O)(=O)CP(=O)(O)O)cc(C)c1Cc1ccc(O)c(C(C)C)c1] | 52 |
| 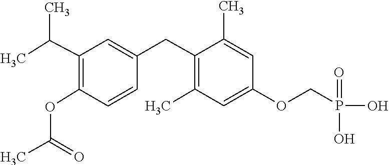 [SMILES: CC(=O)Oc1ccc(Cc2c(C)cc(OCP(=O)(O)O)cc2C)cc1C(C)C] | 58 |
| 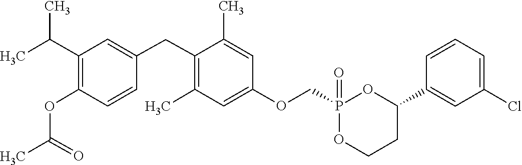 [SMILES: CC(=O)Oc1ccc(Cc2c(C)cc(OC[P@@]3(=O)OCC[C@@H](c4cccc(Cl)c4)O3)cc2C)cc1C(C)C] | 59 |
| 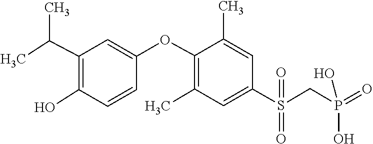 [SMILES: Cc1cc(S(=O)(=O)CP(=O)(O)O)cc(C)c1Oc1ccc(O)c(C(C)C)c1] | 53 |
| 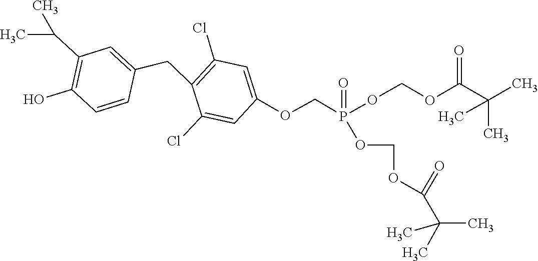 [SMILES: CC(C)c1cc(Cc2c(Cl)cc(OCP(=O)(OCOC(=O)C(C)(C)C)OCOC(=O)C(C)(C)C)cc2Cl)ccc1O] | 12-8 |
| 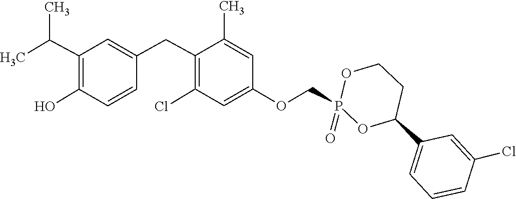 [SMILES: Cc1cc(OC[P@@]2(=O)OCC[C@@H](c3cccc(Cl)c3)O2)cc(Cl)c1Cc1ccc(O)c(C(C)C)c1] | 13-11 |
| 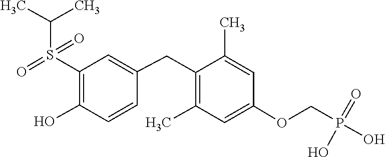 [SMILES: Cc1cc(OCP(=O)(O)O)cc(C)c1Cc1ccc(O)c(S(=O)(=O)C(C)C)c1] | 44 |
| 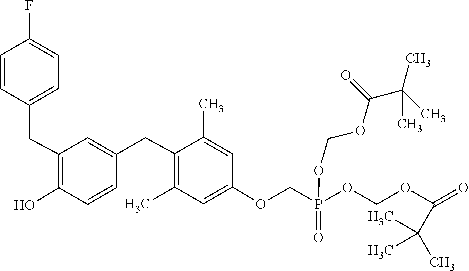 [SMILES: Cc1cc(OCP(=O)(OCOC(=O)C(C)(C)C)OCOC(=O)C(C)(C)C)cc(C)c1Cc1ccc(O)c(Cc2ccc(F)cc2)c1] | 12-6 |
| 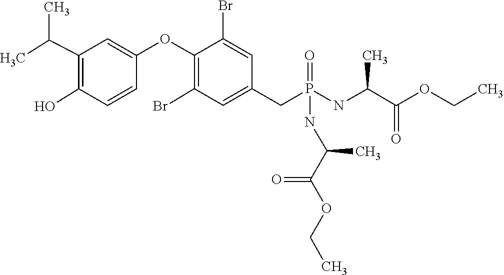 | 15-5 |
| 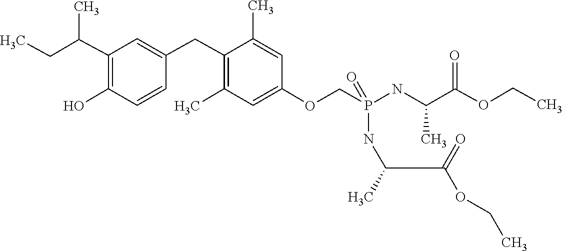 | 15-4 |
| 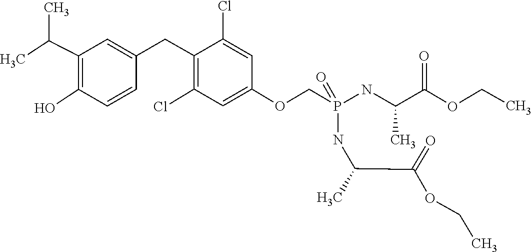 | 15-7 |
| 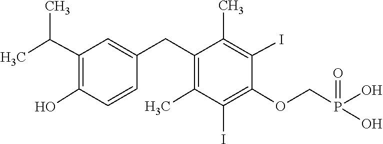 [SMILES: Cc1c(I)c(OCP(=O)(O)O)c(I)c(C)c1Cc1ccc(O)c(C(C)C)c1] | 65-1 |
| 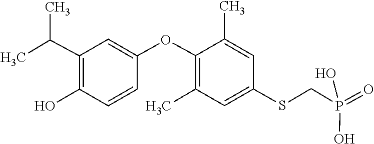 [SMILES: Cc1cc(SCP(=O)(O)O)cc(C)c1Oc1ccc(O)c(C(C)C)c1] | 54 |
| 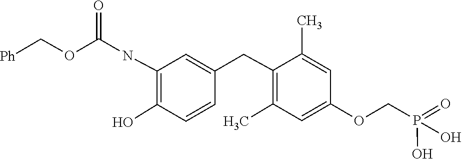 | 50 |
| 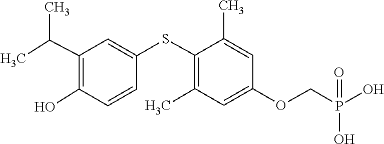 [SMILES: Cc1cc(OCP(=O)(O)O)cc(C)c1Sc1ccc(O)c(C(C)C)c1] | 43 |
| 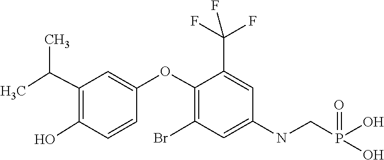 [SMILES: CC(C)c1cc(Oc2c(Br)cc(NCP(=O)(O)O)cc2C(F)(F)F)ccc1O] | 63 |
| 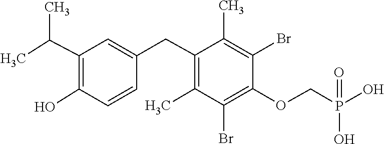 [SMILES: Cc1c(Br)c(OCP(=O)(O)O)c(Br)c(C)c1Cc1ccc(O)c(C(C)C)c1] | 65-2 |
| 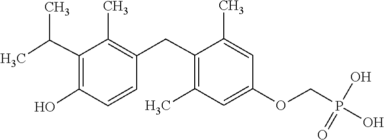 [SMILES: Cc1cc(OCP(=O)(O)O)cc(C)c1Cc1ccc(O)c(C(C)C)c1C] | 7-16 |
| 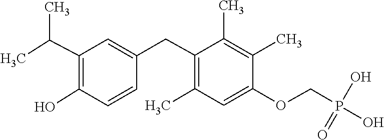 [SMILES: Cc1cc(OCP(=O)(O)O)c(C)c(C)c1Cc1ccc(O)c(C(C)C)c1] | 61 |
| 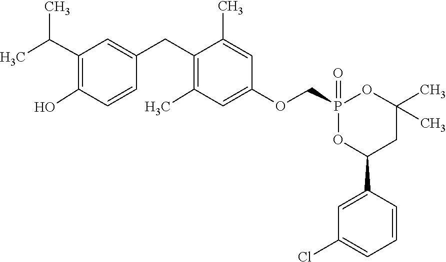 [SMILES: Cc1cc(OC[P@]2(=O)O[C@H](c3cccc(Cl)c3)CC(C)(C)O2)cc(C)c1Cc1ccc(O)c(C(C)C)c1] | 13-13-cis |
| 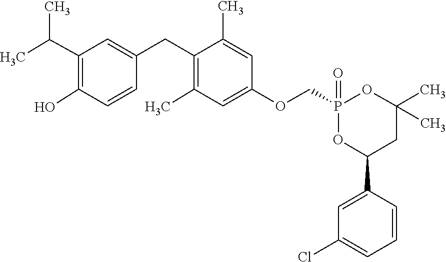 [SMILES: Cc1cc(OC[P@@]2(=O)O[C@H](c3cccc(Cl)c3)CC(C)(C)O2)cc(C)c1Cc1ccc(O)c(C(C)C)c1] | 13-13-trans |
| 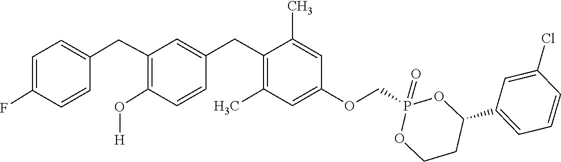 [SMILES: Cc1cc(OC[P@@]2(=O)OCC[C@@H](c3cccc(Cl)c3)O2)cc(C)c1Cc1ccc(O)c(Cc2ccc(F)cc2)c1] | 13-14-cis |
| 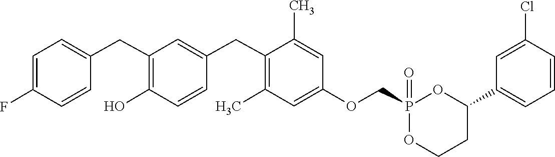 [SMILES: Cc1cc(OC[P@]2(=O)OCC[C@@H](c3cccc(Cl)c3)O2)cc(C)c1Cc1ccc(O)c(Cc2ccc(F)cc2)c1] | 13-14-trans |
| 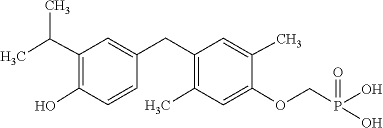 [SMILES: Cc1cc(OCP(=O)(O)O)c(C)cc1Cc1ccc(O)c(C(C)C)c1] | 7-17 |
| 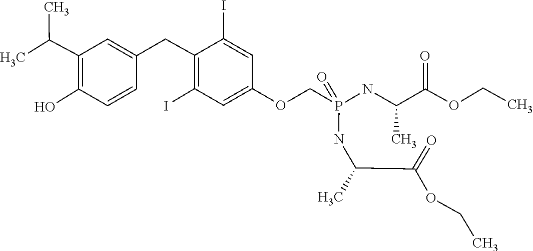 | 15-8 |
| 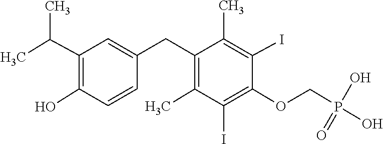 [SMILES: Cc1c(I)c(OCP(=O)(O)O)c(I)c(C)c1Cc1ccc(O)c(C(C)C)c1] | 62 |
| 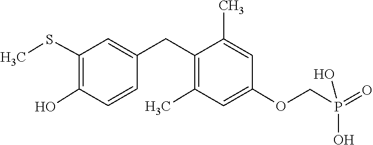 [SMILES: CSc1cc(Cc2c(C)cc(OCP(=O)(O)O)cc2C)ccc1O] | 55 |
| 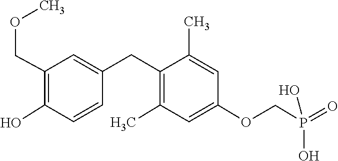 [SMILES: COCc1cc(Cc2c(C)cc(OCP(=O)(O)O)cc2C)ccc1O] | 7-15 |

or pharmaceutically acceptable salts thereof.

The compounds described above may be prepared according to known methods, including those described in U.S. Pat. No. 7,829,552, which is incorporated herein by reference in its entirety. Additional thyroid receptor agonists are described in U.S. Pat. No. 7,514,419; U.S. Application Publication No. 2009/002895; U.S. Application Publication No. 2010/0081634; U.S Application Publication No. 2012/0046364; and PCT Application Publication No. WO 2011/038207, all of which are incorporated herein by reference in their entirety.

In some embodiments, the TR-β agonist is a compound having the structure of Formula (A)

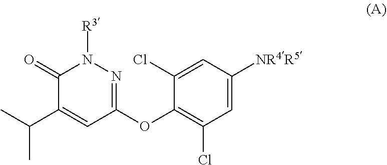

wherein

R^{3′} is H or CH₂R^{a′}, in which R^{a′} is hydroxyl, O-linked amino acid, —OP(O)(OH)₂ or OC(O)R^{b′}, R^{b′} being lower alkyl, alkoxy, alkyl acid, cycloalkyl, aryl, heteroaryl, or —(CH₂)_{n′}-heteroaryl and n′ being 0 or 1;

R^{4′} is H, and R^{5′} is CH₂COOH, C(O)CO₂H, or an ester or amide thereof, or R^{4′} and R^{5′} together are —N═C(R^{c′})—C—(O)—NH—C(O)—; in which R^{c′} is H or cyano; or pharmaceutically acceptable salts thereof.

In some embodiments, the TR-β agonist is selected from the group consisting of:

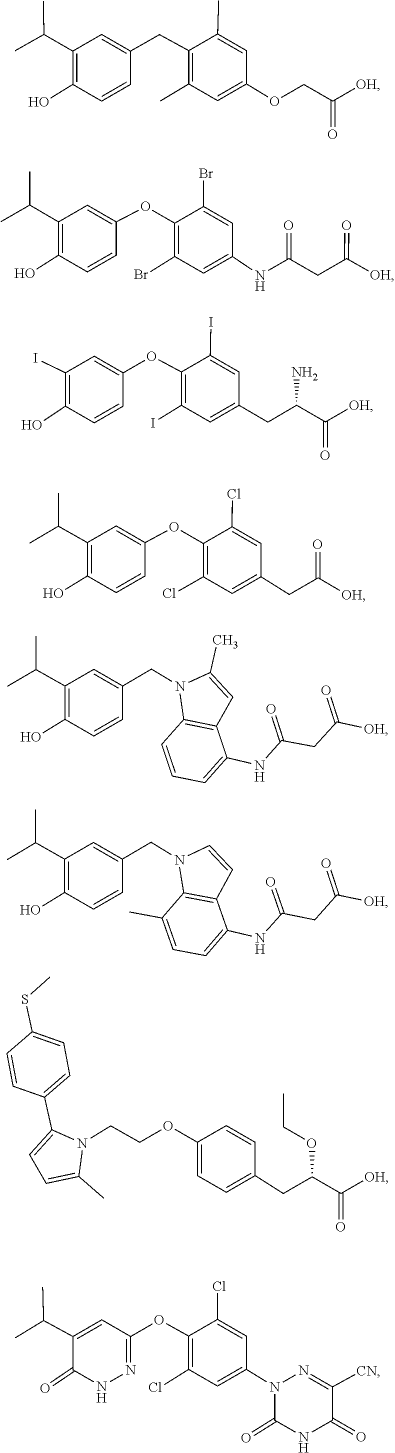

and pharmaceutically acceptable salts thereof.

### Second Pharmaceutical Agents

The TR-β agonist compounds presented herein may be administered in combination with one or more second pharmaceutical agents. In some embodiments, the compounds described above may be administered in combination with one second pharmaceutical agent. In some embodiments, the compounds described above may be administered in combination with two second pharmaceutical agents. In some embodiments, the compounds described above may be administered in combination with three or more second pharmaceutical agents.

In some embodiments, the TR-β agonist compounds presented herein may be administered simultaneously with one or more second pharmaceutical agents. In other embodiments, the compounds of the present disclosure may be administered sequentially with one or more second pharmaceutical agents.

In one aspect, the TR-β agonist compounds presented herein may be administered in combination with a peroxisome proliferator-activated receptor (PPAR) modulator. PPAR modulators are pharmaceutical compounds that may be used e.g., to lower triglyceride levels and blood sugar levels in a subject. PPAR modulators may be classified as PPARα modulators, PPARγ modulators, or PPARδ agonists. In some embodiments, the PPAR modulator may be

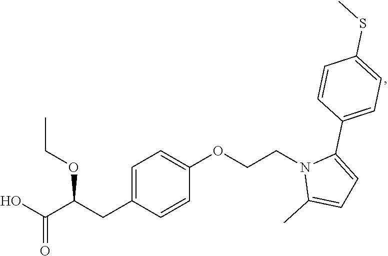 [SMILES: CCO[C@@H](Cc1ccc(OCCn2c(C)ccc2-c2ccc(SC)cc2)cc1)C(=O)O]

In some embodiments, the PPAR modulator may be

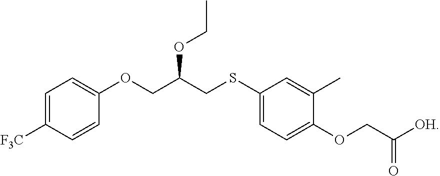 [SMILES: CCO[C@H](COc1ccc(C(F)(F)F)cc1)CSc1ccc(OCC(=O)O)c(C)c1]

In some embodiments, the PPAR modulator may be

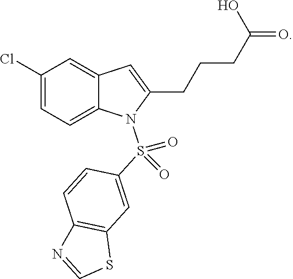 [SMILES: O=C(O)CCCc1cc2cc(Cl)ccc2n1S(=O)(=O)c1ccc2ncsc2c1]

In some embodiments, the PPAR modulator may be

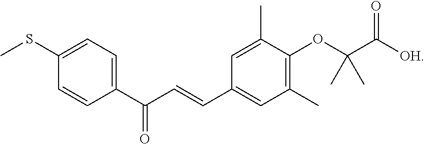 [SMILES: CSc1ccc(C(=O)/C=C/c2cc(C)c(OC(C)(C)C(=O)O)c(C)c2)cc1]

In some embodiments, the PPAR modulator may be a pharmaceutically acceptable salt or prodrug of any of the foregoing.

In some embodiments, the TR-β agonist compounds presented herein may be administered in combination with a fibric acid derivative. Fibric acid derivatives are a class of lipid-lowering drugs that have the ability to lower a subject's lipid profile. In some embodiments, the fibric acid derivative may be fenofibrate. In some embodiments, the fibric acid derivative may be gemfibrozil. In some embodiments, the fibric acid derivative may be fenofibric acid. In some embodiments, the fibric acid derivative may be clofibrate. In some embodiments, the fibric acid derivative may be a pharmaceutically acceptable salt or prodrug of any of the foregoing.

In some embodiments, the TR-β agonist compounds presented herein may be administered in combination with a bile acid receptor modulator. Bile acid receptors include, but are not limited to FXR (farnesoid X receptor) and TGR5. In some embodiments, the bile acid receptor modulator may be

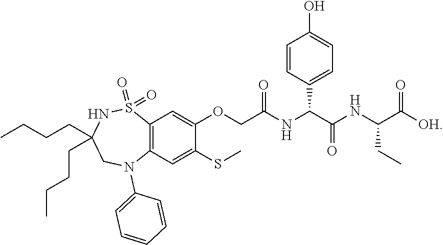 [SMILES: CCCCC1(CCCC)CN(c2ccccc2)c2cc(SC)c(OCC(=O)N[C@@H](C(=O)N[C@@H](CC)C(=O)O)c3ccc(O)cc3)cc2S(=O)(=O)N1]

In some embodiments, the bile acid receptor modulator may be

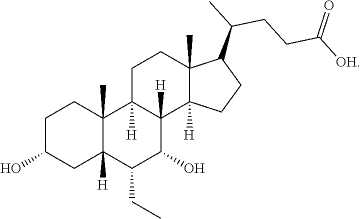

In some embodiments, the bile acid receptor modulator may be

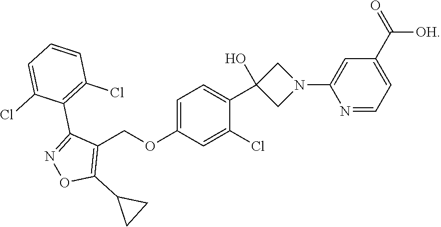 [SMILES: O=C(O)c1ccnc(N2CC(O)(c3ccc(OCc4c(-c5c(Cl)cccc5Cl)noc4C4CC4)cc3Cl)C2)c1]

In some embodiments, the bile acid receptor modulator may be

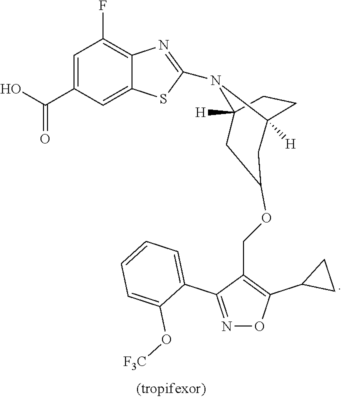 [SMILES: O=C(O)c1cc(F)c2nc(N3[C@@H]4CC[C@H]3CC(OCc3c(-c5ccccc5OC(F)(F)F)noc3C3CC3)C4)sc2c1]

In some embodiments, the bile acid modulator may be a pharmaceutically acceptable salt or prodrug of any of the foregoing.

In some embodiments, the TR-β agonist compounds presented herein may be administered in combination with a bile acid receptor modulator. In some embodiments, the bile acid receptor modulator may be selected from the group consisting of an FXR agonist, an FXR antagonist, a TGR agonist, and a dual FXR/TGR agonist. In some embodiments, the bile receptor acid modulator may be selected from a compound disclosed in Xu, J. Med Chem. 2016, 59, 6553-6579, which is incorporated herein by reference in its entirety, including compounds selected from:

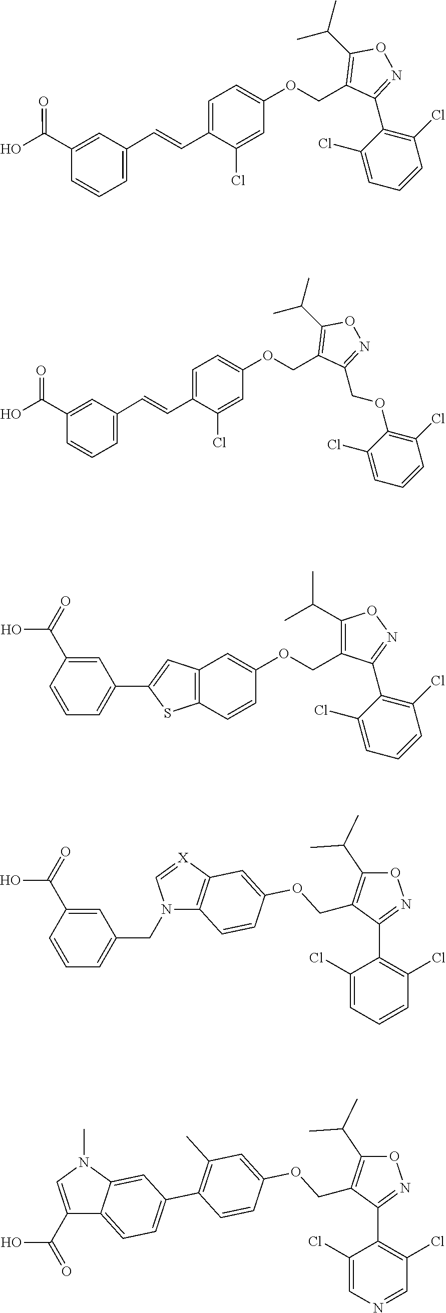

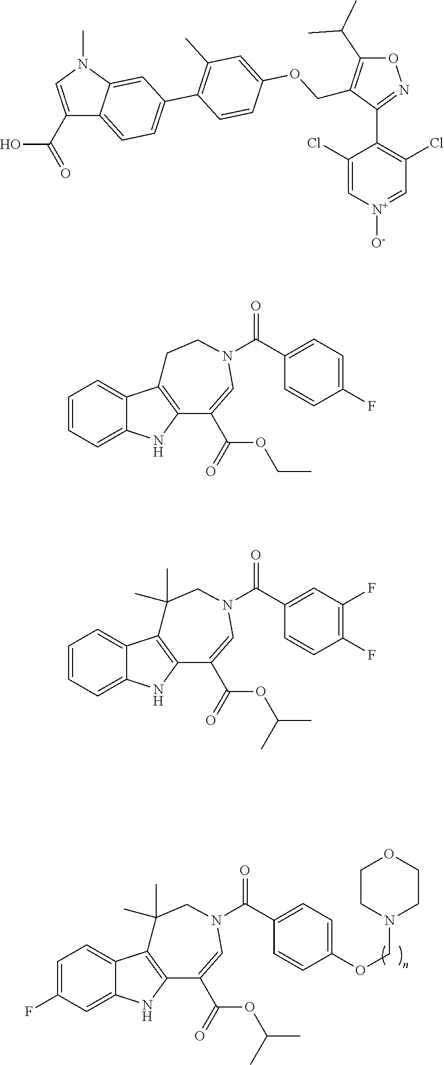

where n is 2 or 3

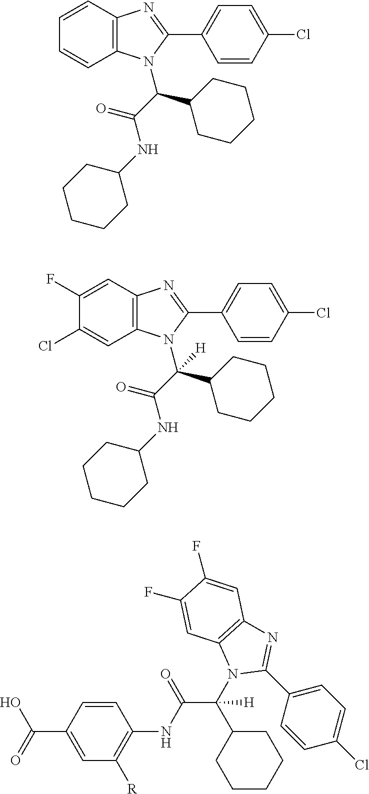

where R is H or F

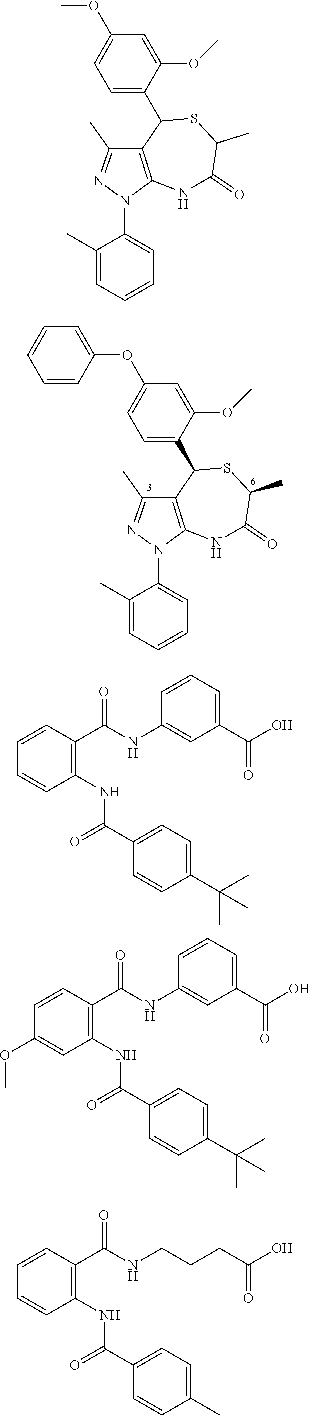

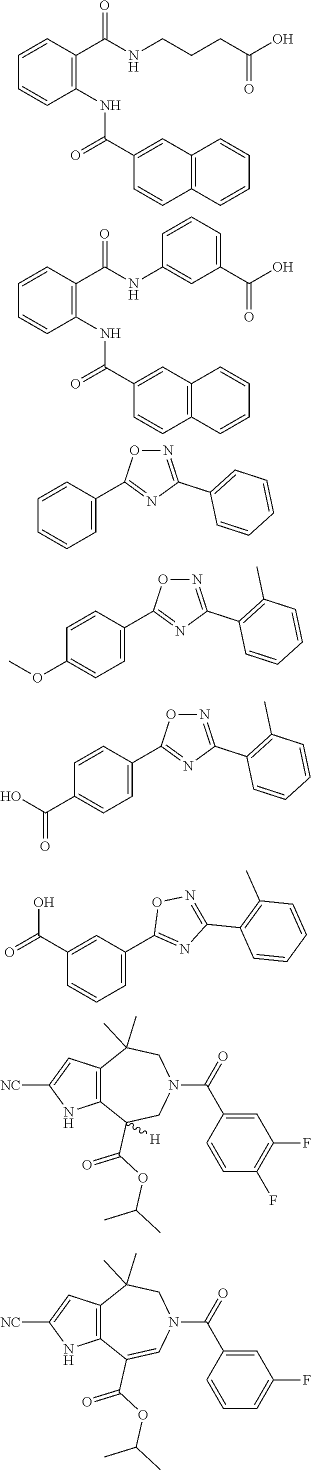

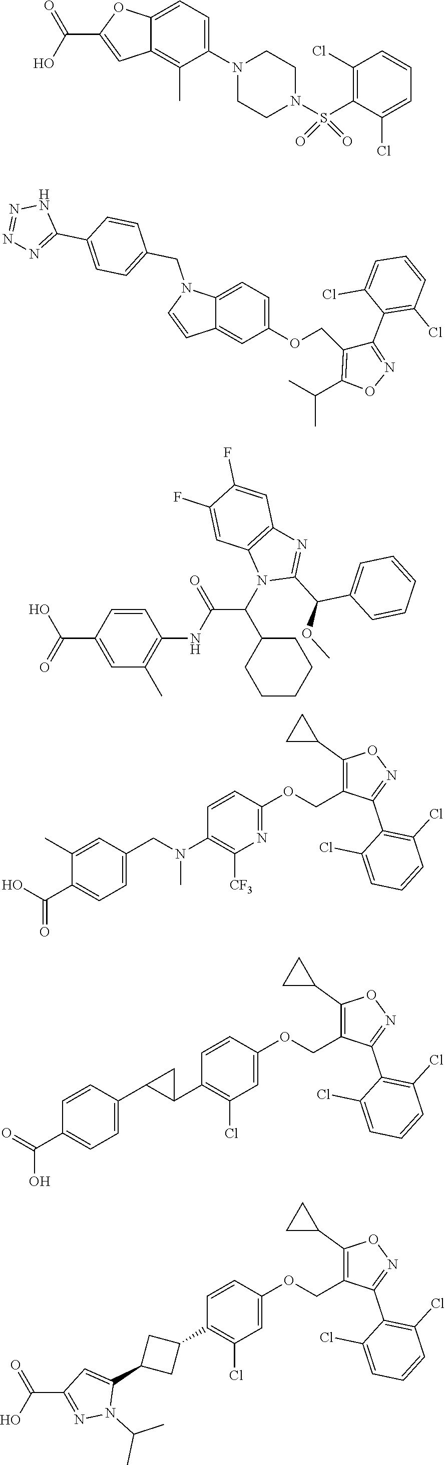

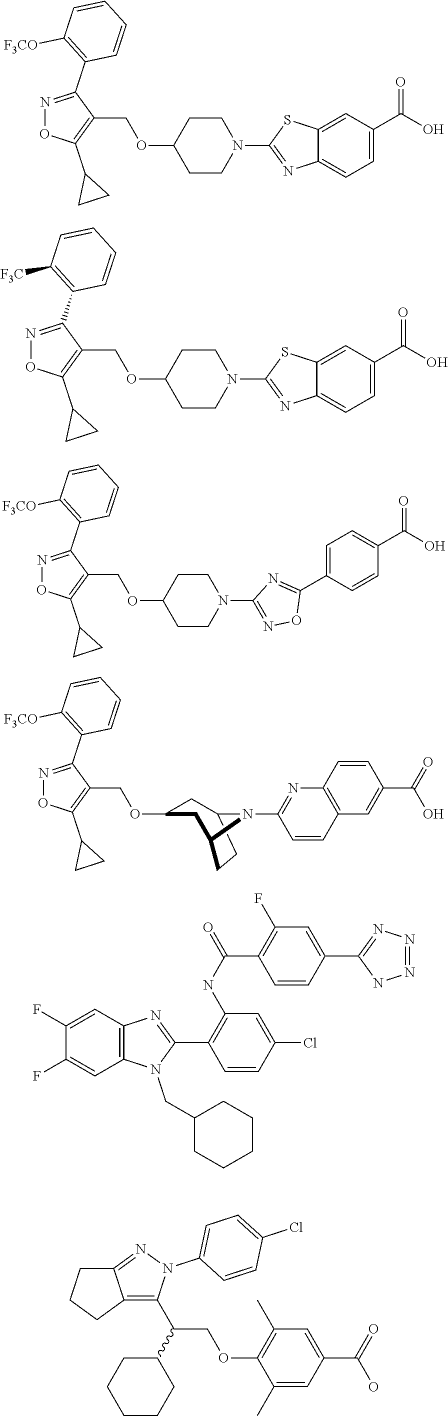

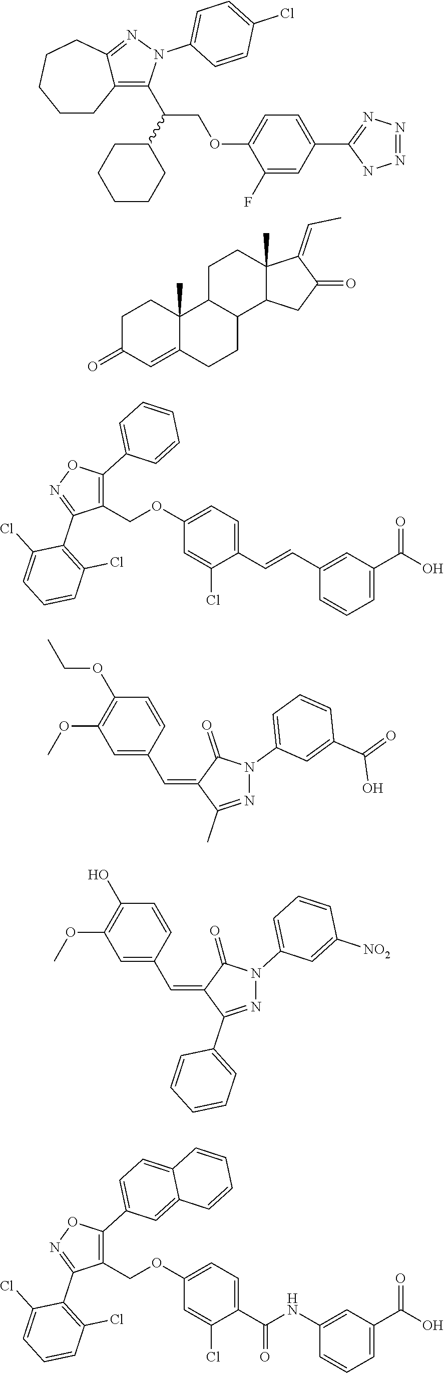

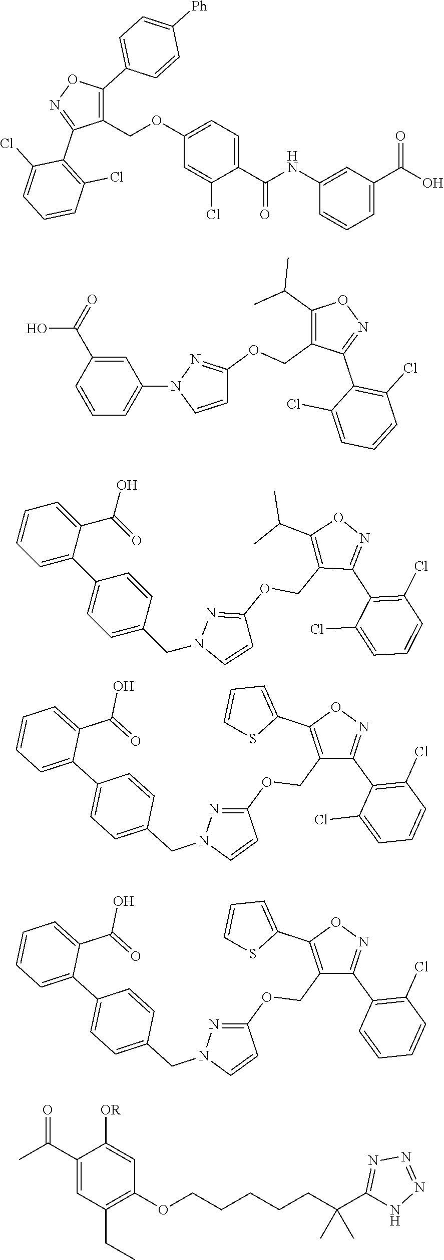

where R is H or benzyl

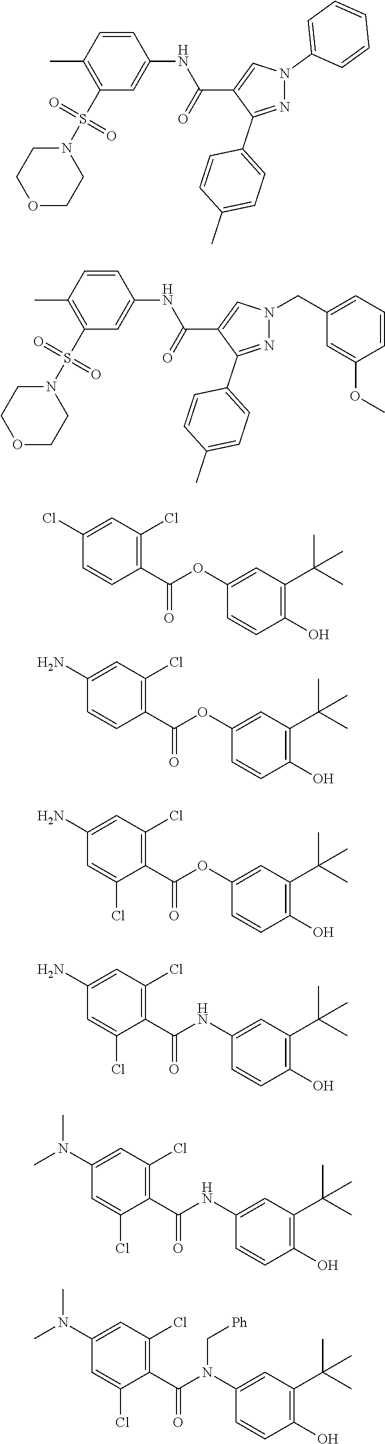

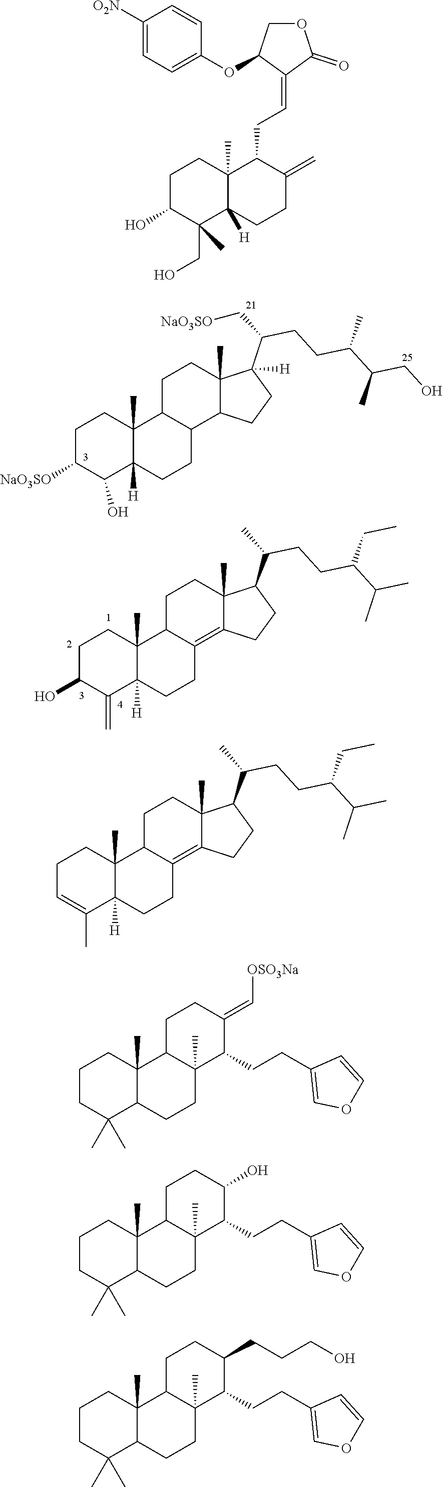

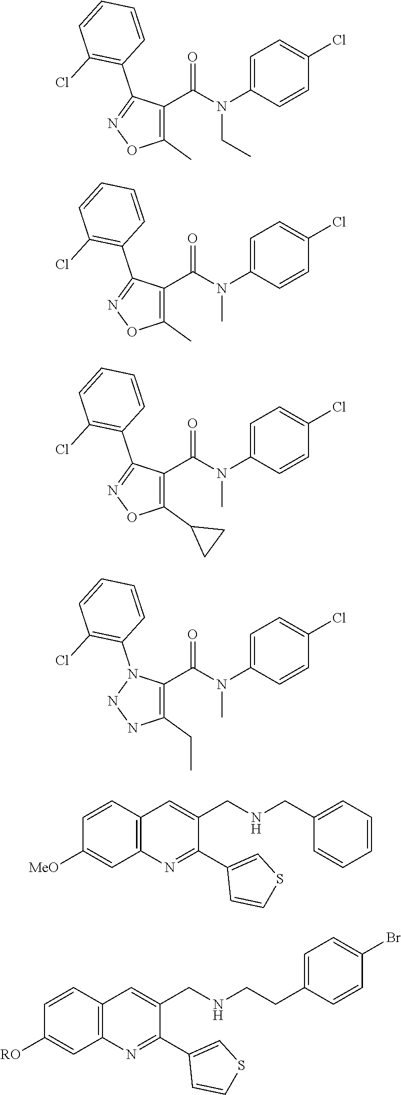

where R is H or methyl

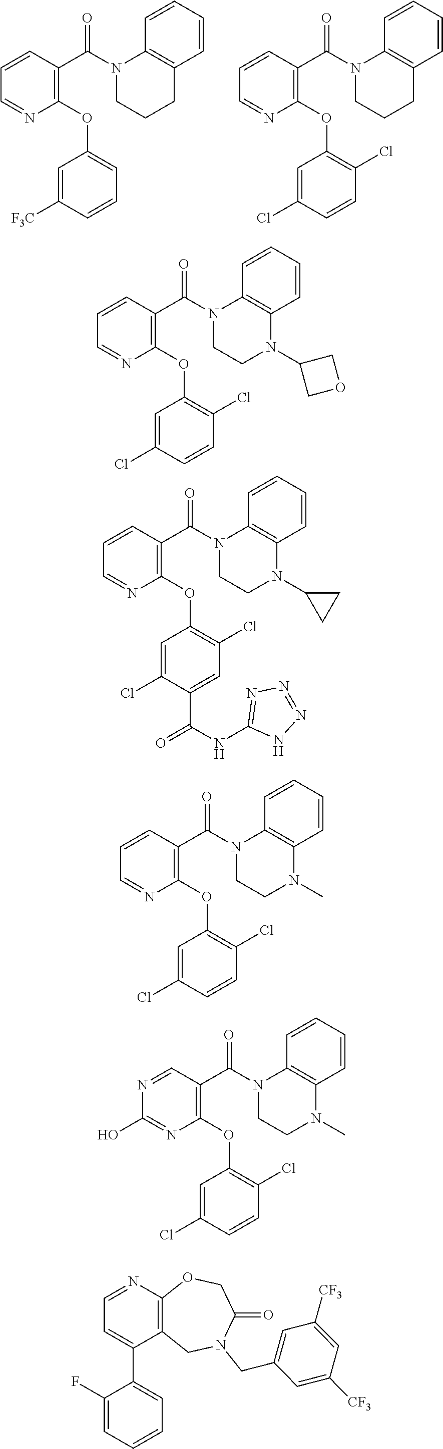

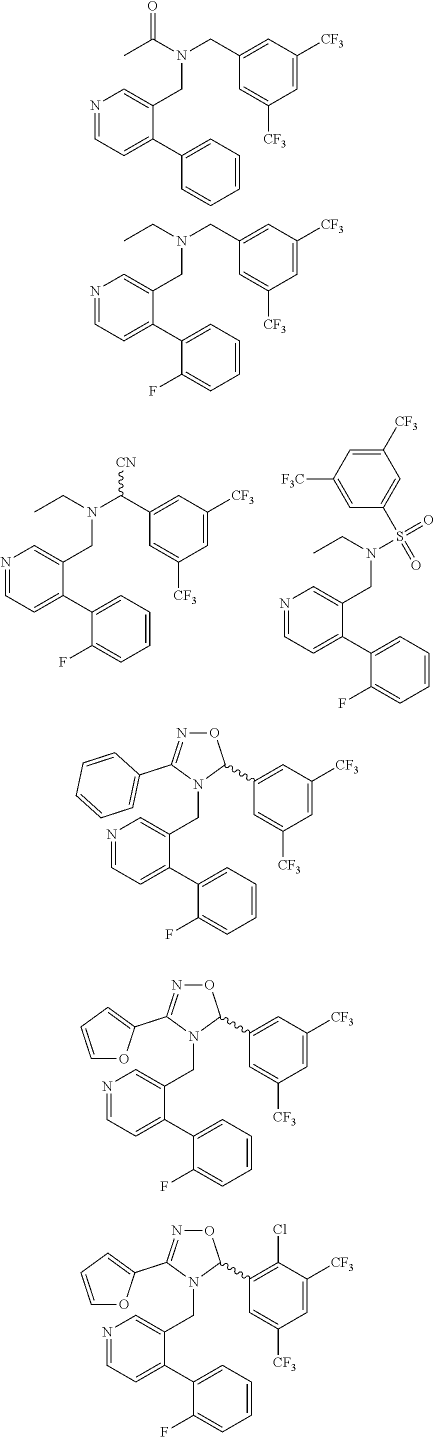

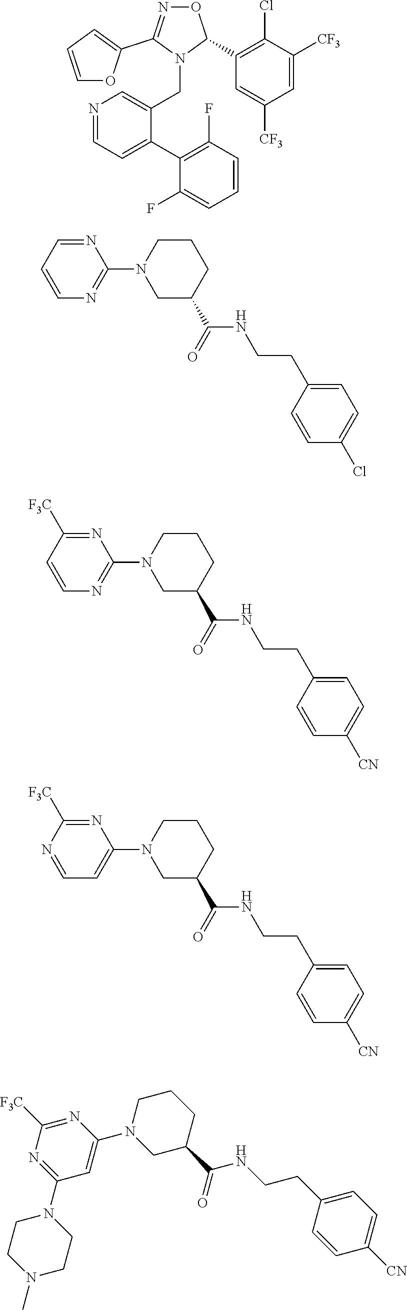

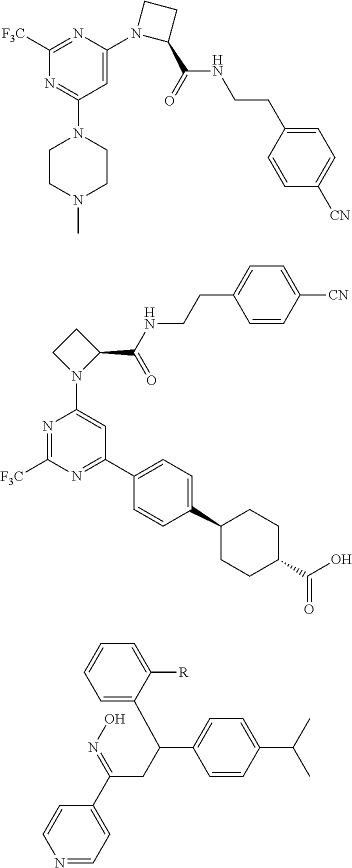

where R is H or methyl

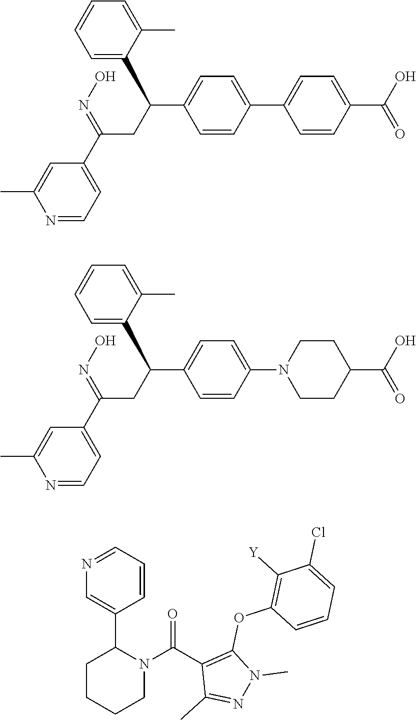

where Y is F or Cl

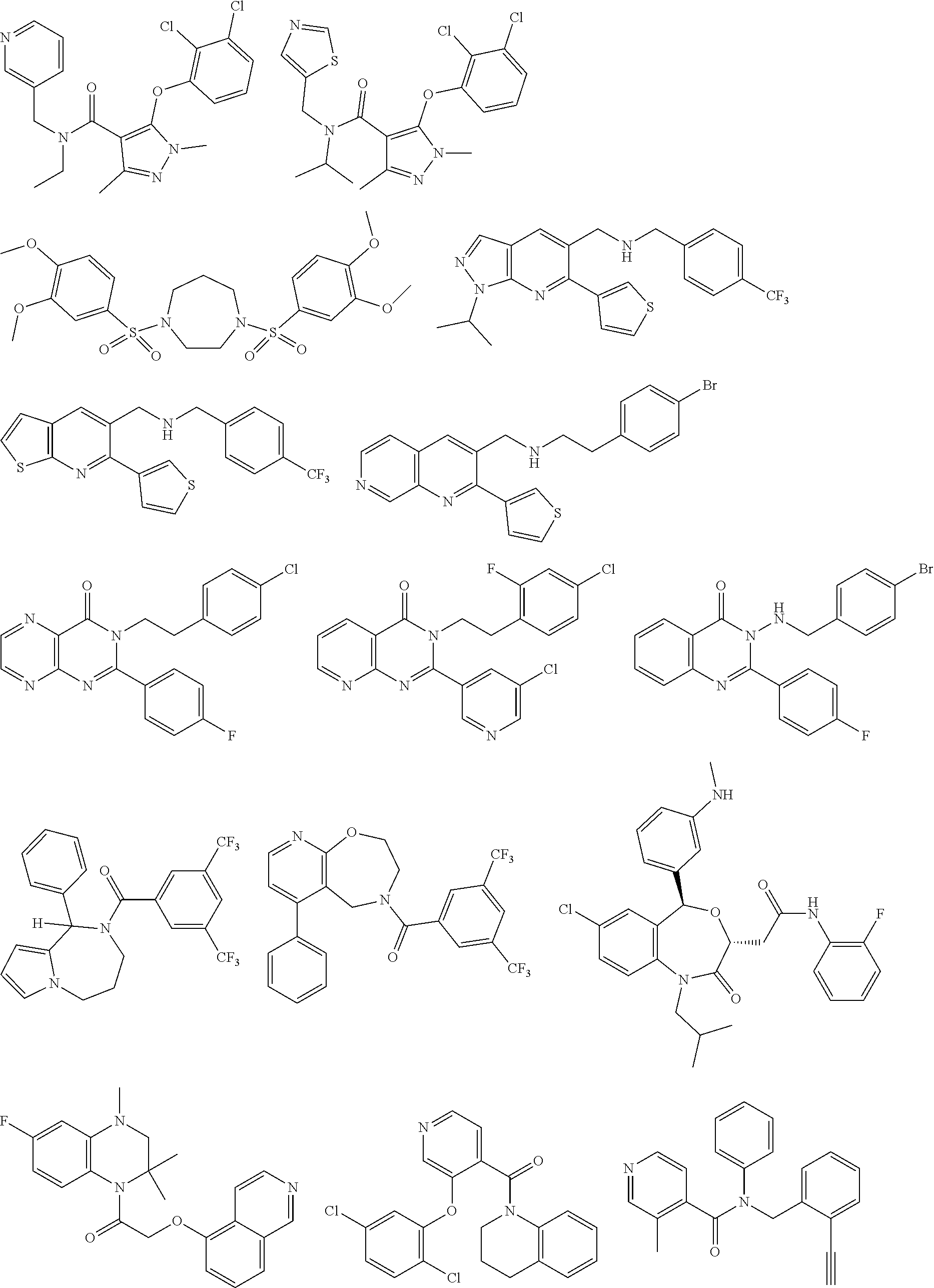

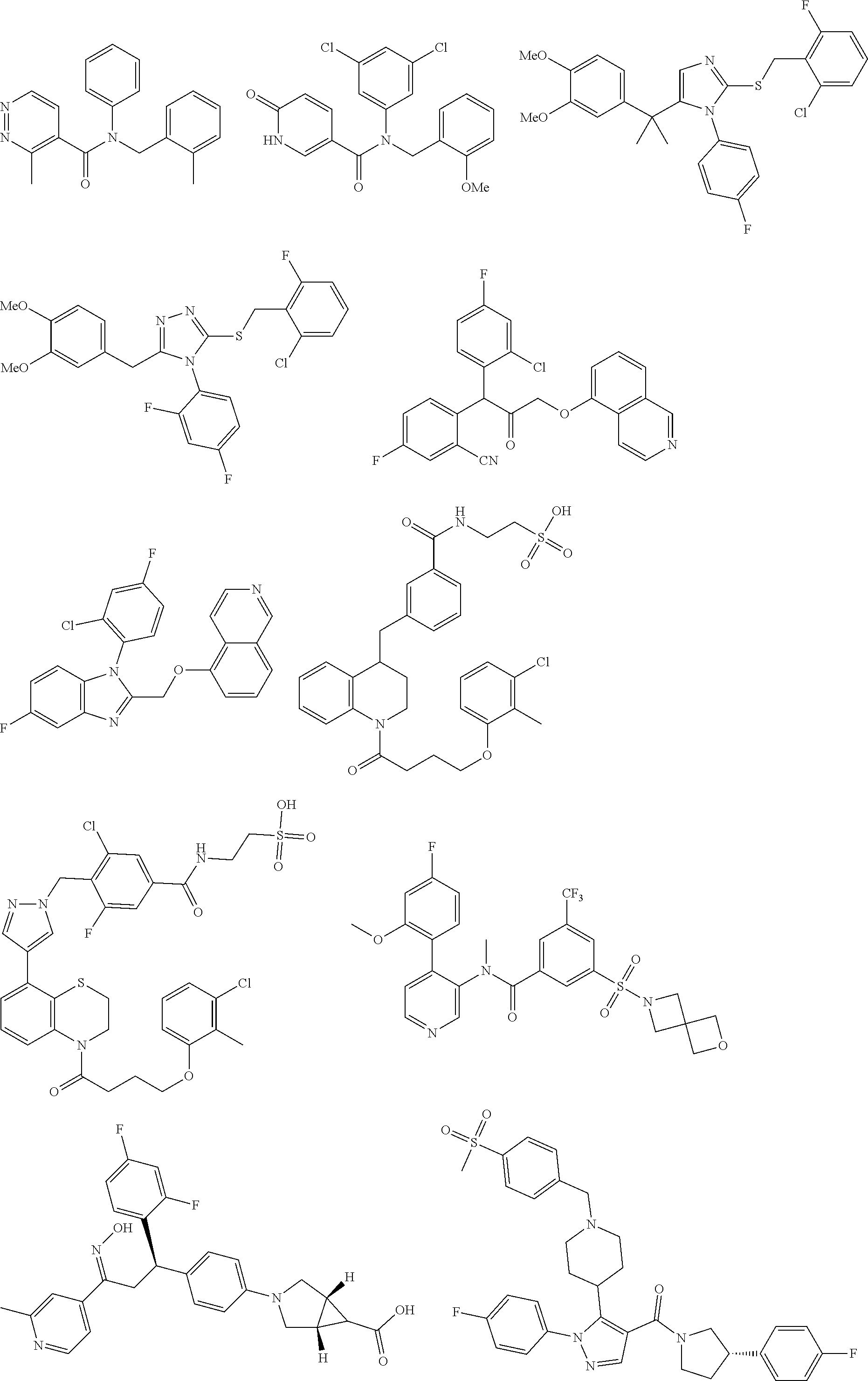

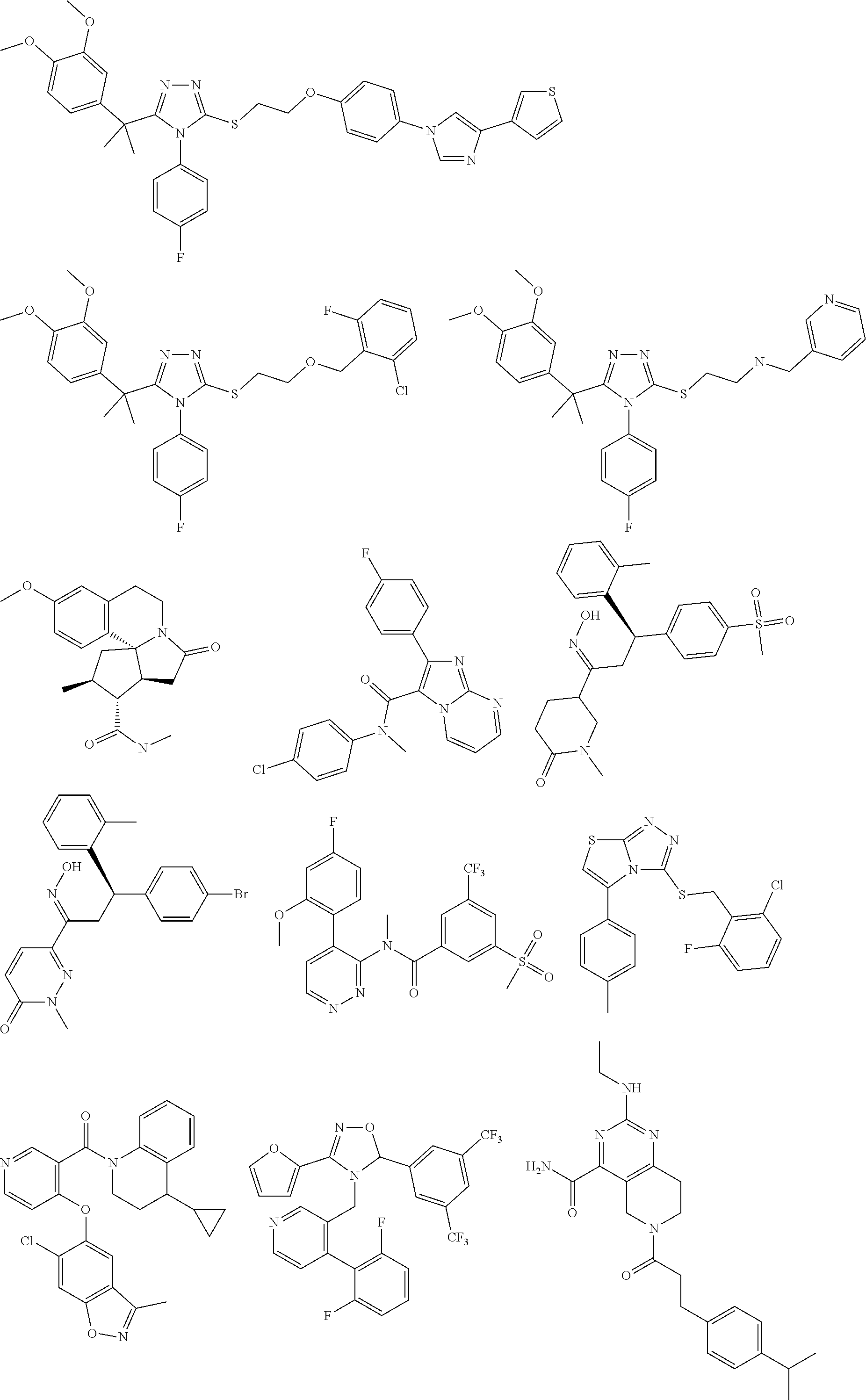

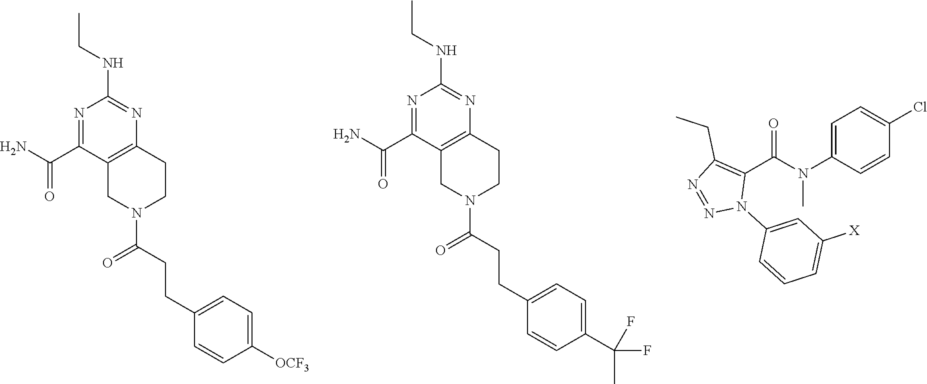

where X is H or OMe

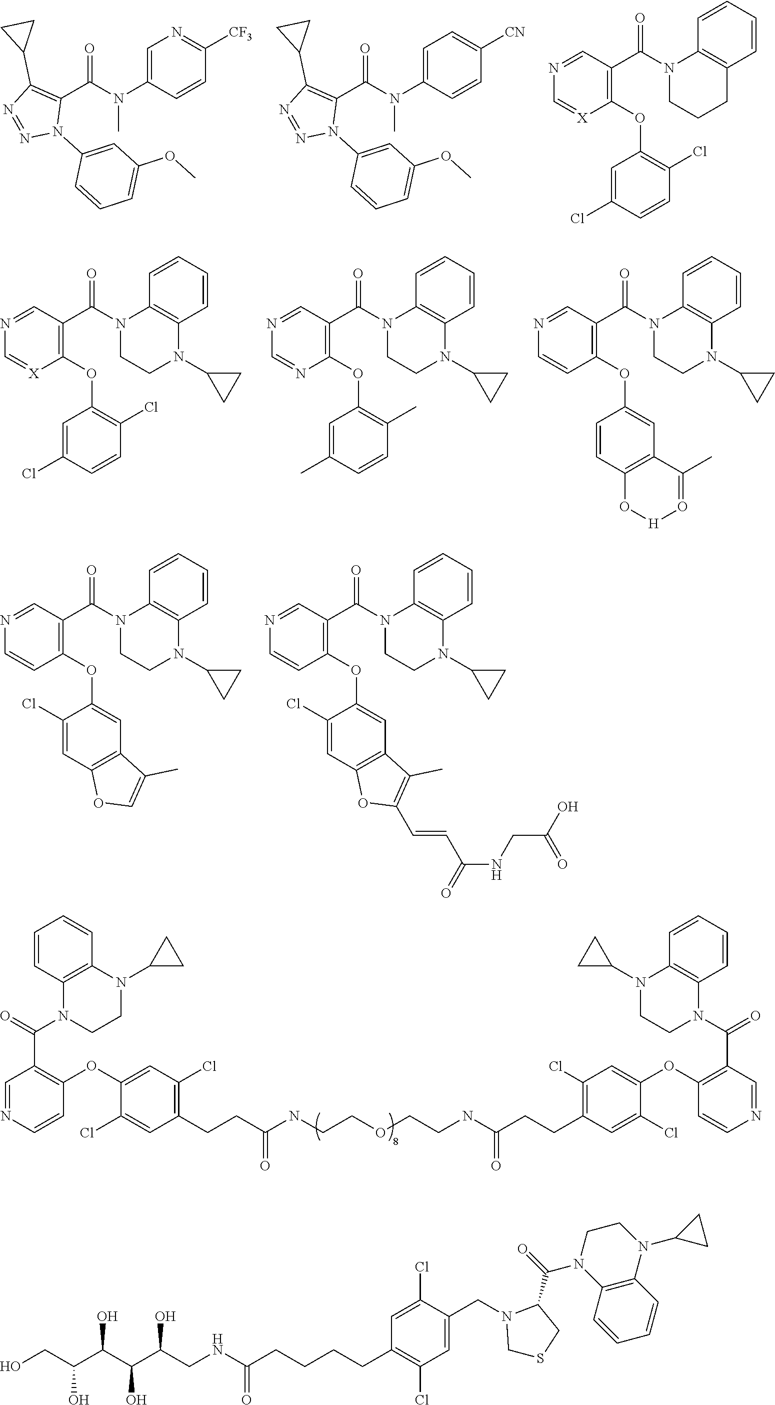

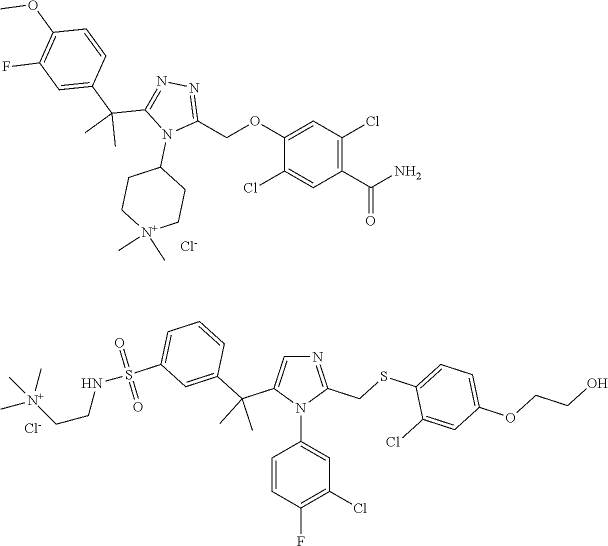

where R is H or ethyl

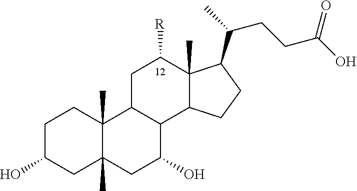

where R is H or OH

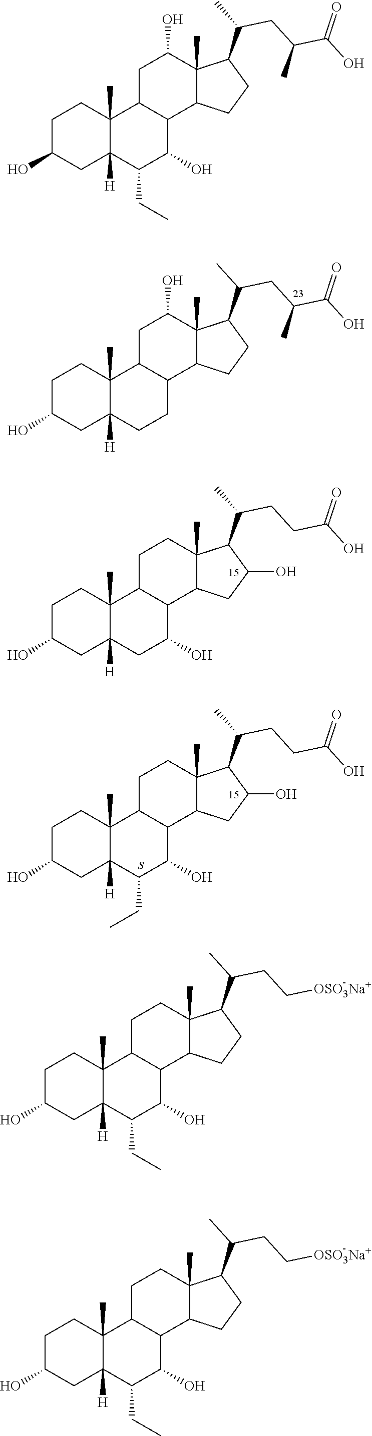

or pharmaceutically acceptable salts of any of the foregoing.

In some embodiments, the TR-β agonist compounds presented herein may be administered in combination with an anti-inflammatory compound. In some embodiments, the anti-inflammatory compound may be

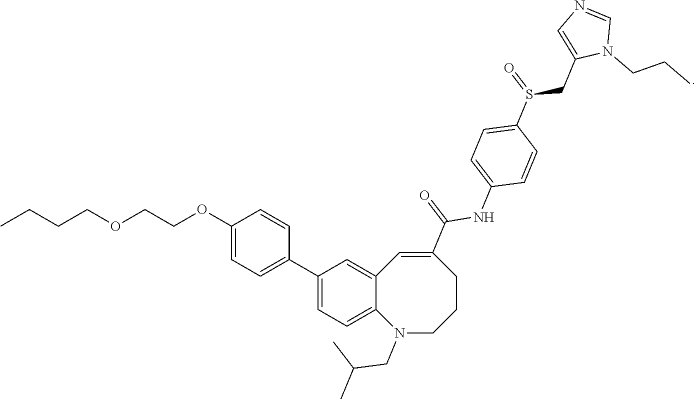 [SMILES: CCCCOCCOc1ccc(-c2ccc3c(c2)/C=C(/C(=O)Nc2ccc([S@@](=O)Cc4cncn4CCC)cc2)CCCN3CC(C)C)cc1]

In some embodiments, the anti-inflammatory compound may be

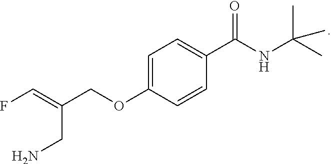 [SMILES: CC(C)(C)NC(=O)c1ccc(OC/C(=C/F)CN)cc1]

In some embodiments, the anti-inflammatory compound may be a poly-clonal or mono-clonal anti-LPS immunoglobulin. In some embodiments, the anti-LPS immunoglobulin may be IMM-124E. In some embodiments, the anti-inflammatory compound may be a pharmaceutically acceptable salt or prodrug of any of the foregoing.

In some embodiments, the second pharmaceutical agent may be an anti-fibrotic compound. In some embodiments, the anti-fibrotic compound may be

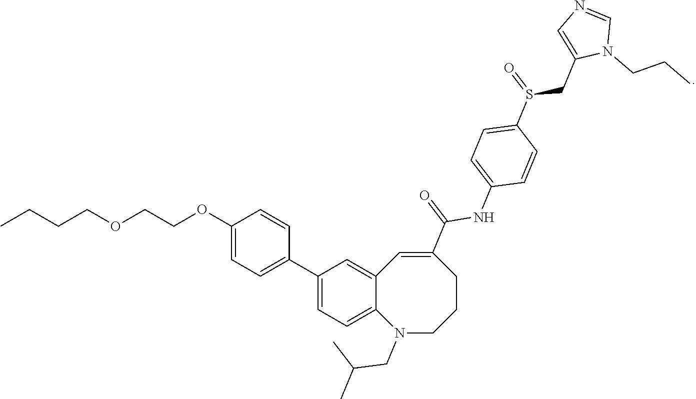 [SMILES: CCCCOCCOc1ccc(-c2ccc3c(c2)/C=C(/C(=O)Nc2ccc([S@@](=O)Cc4cncn4CCC)cc2)CCCN3CC(C)C)cc1]

In some embodiments, the anti-fibrotic compound may be

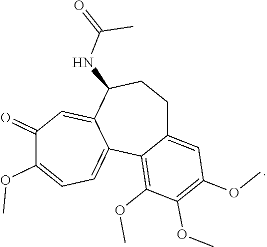

In some embodiments, the anti-fibrotic compound may be a pharmaceutically acceptable salt or prodrug of any of the foregoing.

In some embodiments, TR-β agonist compounds presented herein may be administered in combination with a GLP-1 agonist. GLP-1 are pharmaceutical compounds that may be used e.g., to treat type 2 diabetes in a subject. In some embodiments, the GLP-1 agonist may be dulaglutide. In some embodiments, the GLP-1 agonist may be exenatide. In some embodiments, the GLP-1 agonist may be liraglutide. In some embodiments, the GLP-1 agonist may be albiglutide. In some embodiments, the GLP-1 agonist may be lixisenatide. In some embodiments, the GLP-1 agonist may be semaglutide. In some embodiments, the GLP-1 agonist may be insulin glargine. In some embodiments, the GLP-1 agonist is

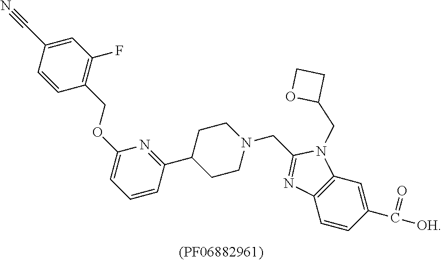 [SMILES: N#Cc1ccc(COc2cccc(C3CCN(Cc4nc5ccc(C(=O)O)cc5n4CC4CCO4)CC3)n2)c(F)c1]

In some embodiments, the GLP-1 agonist may be a pharmaceutically acceptable salt or prodrug of any of the foregoing.

In some embodiments, TR-β agonist compounds presented herein may be administered in combination with a GLP-1 metabolic modulator. In some embodiments, the metabolic modulator may be a thyroid hormone receptor agonist. In other embodiments, the metabolic modulator may be a selective androgen receptor modulator. In some embodiments, the metabolic modulator may be a mitochondrial membrane transport protein modulator. In other embodiments, the metabolic modulator may be a selective estrogen receptor modulator. In some embodiments, the metabolic modulator may be an inhibitor of stearoyl-CoA desaturase 1 (SCD1). In some embodiments, the metabolic modulator may be an inhibitor of dipeptidyl peptidase 4 (DPP-4). In some embodiments, the metabolic modulator may be an inhibitor of sodium glucose cotransporters 1 and/or 2 (SGLT1, SGLT2, or dual SGLT1/SGLT2 inhibitors). In some embodiments, the metabolic modulator may be recombinant fibroblast growth factor 19 (FGF19) or engineered analogs, or recombinant fibroblast growth factor 21 (FGF21) or pegylated variants thereof. In some embodiments, the metabolic modulator may be

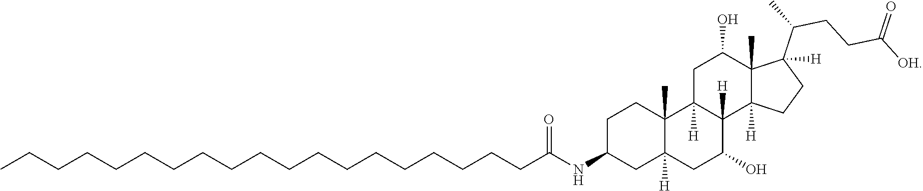

In some embodiments, the metabolic modulator may be

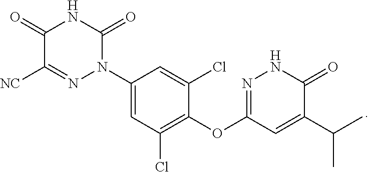 [SMILES: CC(C)c1cc(Oc2c(Cl)cc(-n3nc(C#N)c(=O)[nH]c3=O)cc2Cl)n[nH]c1=O]

In some embodiments, the metabolic modulator may be

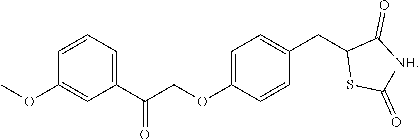 [SMILES: COc1cccc(C(=O)COc2ccc(CC3SC(=O)NC3=O)cc2)c1]

In some embodiments, the metabolic modulator may be a pharmaceutically acceptable salt or prodrug of any of the foregoing.

In some embodiments, TR-β agonist compounds presented herein may be administered in combination with a fish oil derivative. Fish oils contain omega-3-fatty acids, which are polyunsaturated fatty acids (PUFAs) characterized by a double bond three atoms away from the terminal methyl group. They are widely distributed in nature and play an important role in the human diet and in human physiology, particularly with regard to lipid metabolism. In some embodiments, the fish oil derivative may be an omega-3-fatty acid alkyl ester. For example, the fish oil derivative may be an omega-3-fatty acid methyl ester, ethyl ester, n-propyl ester, or isopropyl ester. In some embodiments, the fish oil derivative may be an omega-3-fatty acid triglyceride. In some embodiments, the fish oil derivative may be ethyl (5Z,8Z,11Z,14Z,17Z)-eicosa-5,8,11,14,17-pentaenoate. In some embodiments, the fish oil derivative may be ethyl (4Z,7Z,10Z,13Z,16Z,19Z)-docosa-4,7,10,13,16,19-hexaenoate. In some embodiments, the fish oil derivative may be ethyl (7Z,10Z,13Z,16Z,19Z)-docosapentaenoate. In some embodiments, the fish oil derivative may be ethyl hexadecatrienoate. In some embodiments, the fish oil derivative may be α-linolenic acid ethyl ester. In some embodiments, the fish oil derivative may be ethyl (6Z,9Z,12Z,15Z)-6,9,12,15-octadecatetraenoate. In some embodiments, the fish oil derivative may be ethyl eicosatrienoate. In some embodiments, the fish oil derivative may be ethyl eicosatetraenoate. In some embodiments, the fish oil derivative may be ethyl heneicosapentaenoate. In some embodiments, the fish oil derivative may be ethyl icosapentaenoate. In some embodiments, the fish oil derivative may be ethyl heneicosapentaenoate. In some embodiments, the fish oil derivative may be ethyl tetracosapentaenoate. In some embodiments, the fish oil derivative may be nisinic acid ethyl ester. In some embodiments, the fish oil derivative may be a pharmaceutically acceptable salt or prodrug of any of the foregoing.

### Pharmaceutical Compositions

The TR-β agonist compounds as described above and/or the second pharmaceutical agents described above can be formulated into pharmaceutical compositions for use in treatment of the conditions described herein. Standard pharmaceutical formulation techniques are used, such as those disclosed in Remington's The Science and Practice of Pharmacy, 21st Ed., Lippincott Williams & Wilkins (2005), incorporated herein by reference in its entirety. Accordingly, some embodiments include pharmaceutical compositions comprising: (a) a safe and therapeutically effective amount of a compound described herein, or pharmaceutically acceptable salts thereof; and (b) a pharmaceutically acceptable carrier, diluent, excipient or combination thereof.

In some embodiments, the TR-β agonist compounds provided herein and the second pharmaceutical agents provided herein may be formulated into a single pharmaceutical composition for use in treatment of the conditions described herein. In some embodiments, a formulation comprising the TR-β agonist compounds provided herein may be administered in combination with one or more second pharmaceutical agents provided herein or a pharmaceutical composition comprising one or more second pharmaceutical agents provided herein.

The term “pharmaceutically acceptable carrier” or “pharmaceutically acceptable excipient” includes any and all solvents, diluents, emulsifiers, binders, buffers, dispersion media, coatings, antibacterial and antifungal agents, isotonic and absorption delaying agents and the like, or any other such compound as is known by those of skill in the art to be useful in preparing pharmaceutical formulations. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions. In addition, various adjuvants such as are commonly used in the art may be included. These and other such compounds are described in the literature, e.g., in the Merck Index, Merck & Company, Rahway, N.J. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such as sodium lauryl sulfate; coloring agents; flavoring agents; tableting agents, stabilizers; antioxidants; preservatives; pyrogen-free water; isotonic saline; and phosphate buffer solutions.

The choice of a pharmaceutically-acceptable carrier to be used in conjunction with the subject compound is determined by the way the compound is to be administered.

The compositions described herein are preferably provided in unit dosage form. As used herein, a “unit dosage form” is a composition containing an amount of a compound that is suitable for administration to a subject, in a single dose, according to good medical practice. The preparation of a single or unit dosage form however, does not imply that the dosage form is administered once per day or once per course of therapy. A unit dosage form may comprise a single daily dose or a fractional sub-dose wherein several unit dosage forms are to be administered over the course of a day in order to complete a daily dose. According to the present disclosure, a unit dosage form may be given more or less often that once daily, and may be administered more than once during a course of therapy. Such dosage forms may be administered in any manner consistent with their formulation, including orally, parenterally, and may be administered as an infusion over a period of time (e.g., from about 30 minutes to about 2-6 hours). While single administrations are specifically contemplated, the compositions administered according to the methods described herein may also be administered as a continuous infusion or via an implantable infusion pump.

The methods as described herein may utilize any of a variety of suitable forms for a variety of routes for administration, for example, for oral, nasal, rectal, topical (including transdermal), ocular, intracerebral, intracranial, intrathecal, intra-arterial, intravenous, intramuscular, or other parental routes of administration. The skilled artisan will appreciate that oral and nasal compositions include compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropes, surface-active agents, and encapsulating substances. Optional pharmaceutically-active materials may be included, which do not substantially interfere with the activity of the compound. The amount of carrier employed in conjunction with the compound is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

Various oral dosage forms can be used, including such solid forms as tablets, capsules, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carriers suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid, microcrystalline cellulose, carboxymethyl cellulose, and talc. Tablets may also comprise solubilizers or emulsifiers, such as poloxamers, cremophor/Kolliphor®/Lutrol®, methylcellulose, hydroxypropylmethylcellulose, or others as are known in the art. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which can be readily made by a person skilled in the art.

Peroral (PO) compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Compositions described herein may optionally include other drug actives.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

A liquid composition, which is formulated for topical ophthalmic use, is formulated such that it can be administered topically to the eye. The comfort may be maximized as much as possible, although sometimes formulation considerations (e.g. drug stability) may necessitate less than optimal comfort. In the case that comfort cannot be maximized, the liquid may be formulated such that the liquid is tolerable to the patient for topical ophthalmic use. Additionally, an ophthalmically acceptable liquid may either be packaged for single use, or contain a preservative to prevent contamination over multiple uses.

For ophthalmic application, solutions or medicaments are often prepared using a physiological saline solution as a major vehicle. Ophthalmic solutions may preferably be maintained at a comfortable pH with an appropriate buffer system. The formulations may also contain conventional, pharmaceutically acceptable preservatives, stabilizers and surfactants.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. For many compositions, the pH will be between 4 and 9. Accordingly, buffers include acetate buffers, citrate buffers, phosphate buffers and borate buffers. Acids or bases may be used to adjust the pH of these formulations as needed.

Ophthalmically acceptable antioxidants include, but are not limited to, sodium metabisulfite, sodium thiosulfate, acetylcysteine, butylated hydroxyanisole and butylated hydroxytoluene.

Other excipient components, which may be included in the ophthalmic preparations, are chelating agents. A useful chelating agent is edetate disodium, although other chelating agents may also be used in place or in conjunction with it.

For topical use, including for transdermal administration, creams, ointments, gels, solutions or suspensions, etc., containing the compound disclosed herein are employed. Topical formulations may generally be comprised of a pharmaceutical carrier, co-solvent, emulsifier, penetration enhancer, preservative system, and emollient.

For intravenous administration, the compounds and compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J. Pharm. Sci. Tech. 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol.

The compositions for intravenous administration may be provided to caregivers in the form of one more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration. In embodiments that include administering a combination of a compound described herein and another agent, the combination may be provided to caregivers as a mixture, or the caregivers may mix the two agents prior to administration, or the two agents may be administered separately.

The actual unit dose of the TR-β agonist compounds described herein and/or second pharmaceutical agents described herein depends on the specific compound, and on the condition to be treated. In some embodiments, the dose may be from about 0.01 mg/kg to about 120 mg/kg or more of body weight, from about 0.05 mg/kg or less to about 70 mg/kg, from about 0.1 mg/kg to about 50 mg/kg of body weight, from about 1.0 mg/kg to about 10 mg/kg of body weight, from about 5.0 mg/kg to about 10 mg/kg of body weight, or from about 10.0 mg/kg to about 20.0 mg/kg of body weight. In some embodiments, the dose may be less than 100 mg/kg, 90 mg/kg, 80 mg/kg, 70 mg/kg, 60 mg/kg, 50 mg/kg, 40 mg/kg, 30 mg/kg, 25 mg/kg, 20 mg/kg, 10 mg/kg, 7.5 mg/kg, 6 mg/kg, 5 mg/kg, 4 mg/kg, 3 mg/kg, 2.5 mg/kg, 1 mg/kg, 0.5 mg/kg, 0.1 mg/kg, 0.05 mg/kg or 0.005 mg/kg of body weight. In some embodiments, the actual unit dose is 0.05, 0.07, 0.1, 0.3, 1.0, 3.0, 5.0, 10.0 or 25.0 mg/kg of body weight. Thus, for administration to a 70 kg person, the dosage range would be from about 0.1 mg to 70 mg, from about 1 mg to about 50 mg, from about 0.5 mg to about 10 mg, from about 1 mg to about 10 mg, from about 2.5 mg to about 30 mg, from about 35 mg or less to about 700 mg or more, from about 7 mg to about 600 mg, from about 10 mg to about 500 mg, or from about 20 mg to about 300 mg, or from about 200 mg to about 2000 mg. In some embodiments, the actual unit dose is 0.1 mg. In some embodiments, the actual unit dose is 0.5 mg. In some embodiments, the actual unit dose is 1 mg. In some embodiments, the actual unit dose is 1.5 mg. In some embodiments, the actual unit dose is 2 mg. In some embodiments, the actual unit dose is 2.5 mg. In some embodiments, the actual unit dose is 3 mg. In some embodiments, the actual unit dose is 3.5 mg. In some embodiments, the actual unit dose is 4 mg. In some embodiments, the actual unit dose is 4.5 mg. In some embodiments, the actual unit dose is 5 mg. In some embodiments the actual unit dose is 10 mg. In some embodiments, the actual unit dose is 25 mg. In some embodiments, the actual unit dose is 250 mg or less. In some embodiments, the actual unit dose is 100 mg or less. In some embodiments, the actual unit dose is 70 mg or less.

In some embodiments, the TR-β agonist compound is administered at a dose in the range of about 1-50 mg/m² of the body surface area. In some embodiments, the TR-β agonist compound is administered at a dose in the range of about 1-2, 1-3, 1-4, 1-5, 1-6, 1-7, 1-8, 1-9, 1-10, 1-11, 1-12, 1-13, 1-13.75, 1-14, 1-15, 1-16, 1-17, 1-18, 1-19, 1-20, 1-22.5, 1-25, 1-27.5, 1-30, 1.5-2, 1.5-3, 1.5-4, 1.5-5, 1.5-6, 1.5-7, 1.5-8, 1.5-9, 1.5-10, 1.5-11, 1.5-12, 1.5-13, 1.5-13.75, 1.5-14, 1.5-15, 1.5-16, 1.5-17, 1.5-18, 1.5-19, 1.5-20, 1.5-22.5, 1.5-25, 1.5-27.5, 1.5-30, 2.5-2, 2.5-3, 2.5-4, 2.5-5, 2.5-6, 2.5-7, 2.5-8, 2.5-9, 2.5-10, 2.5-11, 2.5-12, 2.5-13, 2.5-13.75, 2.5-14, 2.5-15, 2.5-16, 2.5-17, 2.5-18, 2.5-19, 2.5-20, 2.5-22.5, 2.5-25, 2.5-27.5, 2.5-30, 2.5-7.5, 3-4, 3-5, 3-6, 3-7, 3-8, 3-9, 3-10, 3-11, 3-12, 3-13, 3-13.75, 3-14, 3-15, 3-16, 3-17, 3-18, 3-19, 3-20, 3-22.5, 3-25, 3-27.5, 3-30, 3.5-6.5, 3.5-13.75, 3.5-15, 2.5-17.5, 4-5, 4-6, 4-7, 4-8, 4-9, 4-10, 4-11, 4-12, 4-13, 4-13.75, 4-14, 4-15, 4-16, 4-17, 4-18, 4-19, 4-20, 4-22.5, 4-25, 4-27.5, 4-30, 5-6, 5-7, 5-8, 5-9, 5-10, 5-11, 5-12, 5-13, 5-13.75, 5-14, 5-15, 5-16, 5-17, 5-18, 5-19, 5-20, 5-22.5, 5-25, 5-27.5, 5-30, 6-7, 6-8, 6-9, 6-10, 6-11, 6-12, 6-13, 6-13.75, 6-14, 6-15, 6-16, 6-17, 6-18, 6-19, 6-20, 6-22.5, 6-25, 6-27.5, 6-30, 7-8, 7-9, 7-10, 7-11, 7-12, 7-13, 7-13.75, 7-14, 7-15, 7-16, 7-17, 7-18, 7-19, 7-20, 7-22.5, 7-25, 7-27.5, 7-30, 7.5-12.5, 7.5-13.5, 7.5-15, 8-9, 8-10, 8-11, 8-12, 8-13, 8-13.75, 8-14, 8-15, 8-16, 8-17, 8-18, 8-19, 8-20, 8-22.5, 8-25, 8-27.5, 8-30, 9-10, 9-11, 9-12, 9-13, 9-13.75, 9-14, 9-15, 9-16, 9-17, 9-18, 9-19, 9-20, 9-22.5, 9-25, 9-27.5, 9-30, 10-11, 10-12, 10-13, 10-13.75, 10-14, 10-15, 10-16, 10-17, 10-18, 10-19, 10-20, 10-22.5, 10-25, 10-27.5, 10-30, 11.5-15.5, 12.5-14.5, 7.5-22.5, 8.5-32.5, 9.5-15.5, 15.5-24.5, 5-35, 17.5-22.5, 22.5-32.5, 25-35, 25.5-24.5, 27.5-32.5, 2-20, 2.5-22.5, or 9.5-21.5 mg/m², of the body surface area. In some embodiments, the TR-β agonist compound is administered at a dose of about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40 mg/m² of the body surface area. In some embodiments, the TR-β agonist compound is administered at a dose less than about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40 mg/m² of the body surface area. In some embodiments, the TR-β agonist compound is administered at a dose greater than about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40, 41, 42, 43, 44, 45, 46, 47, 48, 49, 50 mg/m² of the body surface area.

In some embodiments, the TR-β agonist compound dose is about 0.1 mg-100 mg, 0.1 mg-50 mg, 0.1 mg-20 mg, 0.1 mg-10 mg, 0.5 mg-100 mg, 0.5 mg-50 mg, 0.5 mg-20 mg, 0.5 mg-10 mg, 1 mg-100 mg, 1 mg-50 mg, 1 mg-20 mg, 1 mg-10 mg, 2.5 mg-50 mg, 2.5 mg-20 mg, 2.5 mg-10 mg, or about 2.5 mg-5 mg. In some embodiments, the TR-β agonist compound dose is about 5 mg-300 mg, 5 mg-200 mg, 7.5 mg-200 mg, 10 mg-100 mg, 15 mg-100 mg, 20 mg-100 mg, 30 mg-100 mg, 40 mg-100 mg, 10 mg-80 mg, 15 mg-80 mg, 20 mg-80 mg, 30 mg-80 mg, 40 mg-80 mg, 10 mg-60 mg, 15 mg-60 mg, 20 mg-60 mg, 30 mg-60 mg, or about 40 mg-60 mg. In some embodiments, the TR-β agonist compound administered is about 20 mg-60 mg, 27 mg-60 mg, 20 mg-45 mg, or 27 mg-45 mg. In some embodiments, the TR-β agonist compound administered is about 5 mg-7.5 mg, 5 mg-9 mg, 5 mg-10 mg, 5 mg-12 mg, 5 mg-14 mg, 5 mg-15 mg, 5 mg-16 mg, 5 mg-18 mg, 5 mg-20 mg, 5 mg-22 mg, 5 mg-24 mg, 5 mg-26 mg, 5 mg-28 mg, 5 mg-30 mg, 5 mg-32 mg, 5 mg-34 mg, 5 mg-36 mg, 5 mg-38 mg, 5 mg-40 mg, 5 mg-42 mg, 5 mg-44 mg, 5 mg-46 mg, 5 mg-48 mg, 5 mg-50 mg, 5 mg-52 mg, 5 mg-54 mg, 5 mg-56 mg, 5 mg-58 mg, 5 mg-60 mg, 7 mg-7.7 mg, 7 mg-9 mg, 7 mg-10 mg, 7 mg-12 mg, 7 mg-14 mg, 7 mg-15 mg, 7 mg-16 mg, 7 mg-18 mg, 7 mg-20 mg, 7 mg-22 mg, 7 mg-24 mg, 7 mg-26 mg, 7 mg-28 mg, 7 mg-30 mg, 7 mg-32 mg, 7 mg-34 mg, 7 mg-36 mg, 7 mg-38 mg, 7 mg-40 mg, 7 mg-42 mg, 7 mg-44 mg, 7 mg-46 mg, 7 mg-48 mg, 7 mg-50 mg, 7 mg-52 mg, 7 mg-54 mg, 7 mg-56 mg, 7 mg-58 mg, 7 mg-60 mg, 9 mg-10 mg, 9 mg-12 mg, 9 mg-14 mg, 9 mg-15 mg, 9 mg-16 mg, 9 mg-18 mg, 9 mg-20 mg, 9 mg-22 mg, 9 mg-24 mg, 9 mg-26 mg, 9 mg-28 mg, 9 mg-30 mg, 9 mg-32 mg, 9 mg-34 mg, 9 mg-36 mg, 9 mg-38 mg, 9 mg-40 mg, 9 mg-42 mg, 9 mg-44 mg, 9 mg-46 mg, 9 mg-48 mg, 9 mg-50 mg, 9 mg-52 mg, 9 mg-54 mg, 9 mg-56 mg, 9 mg-58 mg, 9 mg-60 mg, 10 mg-12 mg, 10 mg-14 mg, 10 mg-15 mg, 10 mg-16 mg, 10 mg-18 mg, 10 mg-20 mg, 10 mg-22 mg, 10 mg-24 mg, 10 mg-26 mg, 10 mg-28 mg, 10 mg-30 mg, 10 mg-32 mg, 10 mg-34 mg, 10 mg-36 mg, 10 mg-38 mg, 10 mg-40 mg, 10 mg-42 mg, 10 mg-44 mg, 10 mg-46 mg, 10 mg-48 mg, 10 mg-50 mg, 10 mg-52 mg, 10 mg-54 mg, 10 mg-56 mg, 10 mg-58 mg, 10 mg-60 mg, 12 mg-14 mg, 12 mg-15 mg, 12 mg-16 mg, 12 mg-18 mg, 12 mg-20 mg, 12 mg-22 mg, 12 mg-24 mg, 12 mg-26 mg, 12 mg-28 mg, 12 mg-30 mg, 12 mg-32 mg, 12 mg-34 mg, 12 mg-36 mg, 12 mg-38 mg, 12 mg-40 mg, 12 mg-42 mg, 12 mg-44 mg, 12 mg-46 mg, 12 mg-48 mg, 12 mg-50 mg, 12 mg-52 mg, 12 mg-54 mg, 12 mg-56 mg, 12 mg-58 mg, 12 mg-60 mg, 15 mg-16 mg, 15 mg-18 mg, 15 mg-20 mg, 15 mg-22 mg, 15 mg-24 mg, 15 mg-26 mg, 15 mg-28 mg, 15 mg-30 mg, 15 mg-32 mg, 15 mg-34 mg, 15 mg-36 mg, 15 mg-38 mg, 15 mg-40 mg, 15 mg-42 mg, 15 mg-44 mg, 15 mg-46 mg, 15 mg-48 mg, 15 mg-50 mg, 15 mg-52 mg, 15 mg-54 mg, 15 mg-56 mg, 15 mg-58 mg, 15 mg-60 mg, 17 mg-18 mg, 17 mg-20 mg, 17 mg-22 mg, 17 mg-24 mg, 17 mg-26 mg, 17 mg-28 mg, 17 mg-30 mg, 17 mg-32 mg, 17 mg-34 mg, 17 mg-36 mg, 17 mg-38 mg, 17 mg-40 mg, 17 mg-42 mg, 17 mg-44 mg, 17 mg-46 mg, 17 mg-48 mg, 17 mg-50 mg, 17 mg-52 mg, 17 mg-54 mg, 17 mg-56 mg, 17 mg-58 mg, 17 mg-60 mg, 20 mg-22 mg, 20 mg-24 mg, 20 mg-26 mg, 20 mg-28 mg, 20 mg-30 mg, 20 mg-32 mg, 20 mg-34 mg, 20 mg-36 mg, 20 mg-38 mg, 20 mg-40 mg, 20 mg-42 mg, 20 mg-44 mg, 20 mg-46 mg, 20 mg-48 mg, 20 mg-50 mg, 20 mg-52 mg, 20 mg-54 mg, 20 mg-56 mg, 20 mg-58 mg, 20 mg-60 mg, 22 mg-24 mg, 22 mg-26 mg, 22 mg-28 mg, 22 mg-30 mg, 22 mg-32 mg, 22 mg-34 mg, 22 mg-36 mg, 22 mg-38 mg, 22 mg-40 mg, 22 mg-42 mg, 22 mg-44 mg, 22 mg-46 mg, 22 mg-48 mg, 22 mg-50 mg, 22 mg-52 mg, 22 mg-54 mg, 22 mg-56 mg, 22 mg-58 mg, 22 mg-60 mg, 25 mg-26 mg, 25 mg-28 mg, 25 mg-30 mg, 25 mg-32 mg, 25 mg-34 mg, 25 mg-36 mg, 25 mg-38 mg, 25 mg-40 mg, 25 mg-42 mg, 25 mg-44 mg, 25 mg-46 mg, 25 mg-48 mg, 25 mg-50 mg, 25 mg-52 mg, 25 mg-54 mg, 25 mg-56 mg, 25 mg-58 mg, 25 mg-60 mg, 27 mg-28 mg, 27 mg-30 mg, 27 mg-32 mg, 27 mg-34 mg, 27 mg-36 mg, 27 mg-38 mg, 27 mg-40 mg, 27 mg-42 mg, 27 mg-44 mg, 27 mg-46 mg, 27 mg-48 mg, 27 mg-50 mg, 27 mg-52 mg, 27 mg-54 mg, 27 mg-56 mg, 27 mg-58 mg, 27 mg-60 mg, 30 mg-32 mg, 30 mg-34 mg, 30 mg-36 mg, 30 mg-38 mg, 30 mg-40 mg, 30 mg-42 mg, 30 mg-44 mg, 30 mg-46 mg, 30 mg-48 mg, 30 mg-50 mg, 30 mg-52 mg, 30 mg-54 mg, 30 mg-56 mg, 30 mg-58 mg, 30 mg-60 mg, 33 mg-34 mg, 33 mg-36 mg, 33 mg-38 mg, 33 mg-40 mg, 33 mg-42 mg, 33 mg-44 mg, 33 mg-46 mg, 33 mg-48 mg, 33 mg-50 mg, 33 mg-52 mg, 33 mg-54 mg, 33 mg-56 mg, 33 mg-58 mg, 33 mg-60 mg, 36 mg-38 mg, 36 mg-40 mg, 36 mg-42 mg, 36 mg-44 mg, 36 mg-46 mg, 36 mg-48 mg, 36 mg-50 mg, 36 mg-52 mg, 36 mg-54 mg, 36 mg-56 mg, 36 mg-58 mg, 36 mg-60 mg, 40 mg-42 mg, 40 mg-44 mg, 40 mg-46 mg, 40 mg-48 mg, 40 mg-50 mg, 40 mg-52 mg, 40 mg-54 mg, 40 mg-56 mg, 40 mg-58 mg, 40 mg-60 mg, 43 mg-46 mg, 43 mg-48 mg, 43 mg-50 mg, 43 mg-52 mg, 43 mg-54 mg, 43 mg-56 mg, 43 mg-58 mg, 42 mg-60 mg, 45 mg-48 mg, 45 mg-50 mg, 45 mg-52 mg, 45 mg-54 mg, 45 mg-56 mg, 45 mg-58 mg, 45 mg-60 mg, 48 mg-50 mg, 48 mg-52 mg, 48 mg-54 mg, 48 mg-56 mg, 48 mg-58 mg, 48 mg-60 mg, 50 mg-52 mg, 50 mg-54 mg, 50 mg-56 mg, 50 mg-58 mg, 50 mg-60 mg, 52 mg-54 mg, 52 mg-56 mg, 52 mg-58 mg, or 52 mg-60 mg. In some embodiments, the TR-β agonist compound dose is greater than about 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, or about 200 mg. In some embodiments, the TR-β agonist compound dose is about less than about 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, or about 200 mg. In some embodiments, TR-β agonist compound dose is about 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, about 200 mg, about 225 mg, about 250 mg, about 275 mg, or about 300 mg.

In some embodiments, the second pharmaceutical agent is administered at a dose in the range of about 1-50 mg/m² of the body surface area. In some embodiments, the second pharmaceutical agent is administered at a dose in the range of about 1-2, 1-3, 1-4, 1-5, 1-6, 1-7, 1-8, 1-9, 1-10, 1-11, 1-12, 1-13, 1-13.75, 1-14, 1-15, 1-16, 1-17, 1-18, 1-19, 1-20, 1-22.5, 1-25, 1-27.5, 1-30, 1.5-2, 1.5-3, 1.5-4, 1.5-5, 1.5-6, 1.5-7, 1.5-8, 1.5-9, 1.5-10, 1.5-11, 1.5-12, 1.5-13, 1.5-13.75, 1.5-14, 1.5-15, 1.5-16, 1.5-17, 1.5-18, 1.5-19, 1.5-20, 1.5-22.5, 1.5-25, 1.5-27.5, 1.5-30, 2.5-2, 2.5-3, 2.5-4, 2.5-5, 2.5-6, 2.5-7, 2.5-8, 2.5-9, 2.5-10, 2.5-11, 2.5-12, 2.5-13, 2.5-13.75, 2.5-14, 2.5-15, 2.5-16, 2.5-17, 2.5-18, 2.5-19, 2.5-20, 2.5-22.5, 2.5-25, 2.5-27.5, 2.5-30, 2.5-7.5, 3-4, 3-5, 3-6, 3-7, 3-8, 3-9, 3-10, 3-11, 3-12, 3-13, 3-13.75, 3-14, 3-15, 3-16, 3-17, 3-18, 3-19, 3-20, 3-22.5, 3-25, 3-27.5, 3-30, 3.5-6.5, 3.5-13.75, 3.5-15, 2.5-17.5, 4-5, 4-6, 4-7, 4-8, 4-9, 4-10, 4-11, 4-12, 4-13, 4-13.75, 4-14, 4-15, 4-16, 4-17, 4-18, 4-19, 4-20, 4-22.5, 4-25, 4-27.5, 4-30, 5-6, 5-7, 5-8, 5-9, 5-10, 5-11, 5-12, 5-13, 5-13.75, 5-14, 5-15, 5-16, 5-17, 5-18, 5-19, 5-20, 5-22.5, 5-25, 5-27.5, 5-30, 6-7, 6-8, 6-9, 6-10, 6-11, 6-12, 6-13, 6-13.75, 6-14, 6-15, 6-16, 6-17, 6-18, 6-19, 6-20, 6-22.5, 6-25, 6-27.5, 6-30, 7-8, 7-9, 7-10, 7-11, 7-12, 7-13, 7-13.75, 7-14, 7-15, 7-16, 7-17, 7-18, 7-19, 7-20, 7-22.5, 7-25, 7-27.5, 7-30, 7.5-12.5, 7.5-13.5, 7.5-15, 8-9, 8-10, 8-11, 8-12, 8-13, 8-13.75, 8-14, 8-15, 8-16, 8-17, 8-18, 8-19, 8-20, 8-22.5, 8-25, 8-27.5, 8-30, 9-10, 9-11, 9-12, 9-13, 9-13.75, 9-14, 9-15, 9-16, 9-17, 9-18, 9-19, 9-20, 9-22.5, 9-25, 9-27.5, 9-30, 10-11, 10-12, 10-13, 10-13.75, 10-14, 10-15, 10-16, 10-17, 10-18, 10-19, 10-20, 10-22.5, 10-25, 10-27.5, 10-30, 11.5-15.5, 12.5-14.5, 7.5-22.5, 8.5-32.5, 9.5-15.5, 15.5-24.5, 5-35, 17.5-22.5, 22.5-32.5, 25-35, 25.5-24.5, 27.5-32.5, 2-20, 2.5-22.5, or 9.5-21.5 mg/m², of the body surface area. In some embodiments, the second pharmaceutical agent is administered at a dose of about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40 mg/m² of the body surface area. In some embodiments, the second pharmaceutical agent is administered at a dose less than about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40 mg/m² of the body surface area. In some embodiments, the second pharmaceutical agent is administered at a dose greater than about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40, 41, 42, 43, 44, 45, 46, 47, 48, 49, 50 mg/m² of the body surface area.

In some embodiments, the second pharmaceutical agent dose is about 5 mg-300 mg, 5 mg-200 mg, 7.5 mg-200 mg, 10 mg-100 mg, 15 mg-100 mg, 20 mg-100 mg, 30 mg-100 mg, 40 mg-100 mg, 10 mg-80 mg, 15 mg-80 mg, 20 mg-80 mg, 30 mg-80 mg, 40 mg-80 mg, 10 mg-60 mg, 15 mg-60 mg, 20 mg-60 mg, 30 mg-60 mg, or about 40 mg-60 mg. In some embodiments, the second pharmaceutical agent dose administered is about 20 mg-60 mg, 27 mg-60 mg, 20 mg-45 mg, or 27 mg-45 mg. In some embodiments, the second pharmaceutical agent dose administered is about 5 mg-7.5 mg, 5 mg-9 mg, 5 mg-10 mg, 5 mg-12 mg, 5 mg-14 mg, 5 mg-15 mg, 5 mg-16 mg, 5 mg-18 mg, 5 mg-20 mg, 5 mg-22 mg, 5 mg-24 mg, 5 mg-26 mg, 5 mg-28 mg, 5 mg-30 mg, 5 mg-32 mg, 5 mg-34 mg, 5 mg-36 mg, 5 mg-38 mg, 5 mg-40 mg, 5 mg-42 mg, 5 mg-44 mg, 5 mg-46 mg, 5 mg-48 mg, 5 mg-50 mg, 5 mg-52 mg, 5 mg-54 mg, 5 mg-56 mg, 5 mg-58 mg, 5 mg-60 mg, 7 mg-7.7 mg, 7 mg-9 mg, 7 mg-10 mg, 7 mg-12 mg, 7 mg-14 mg, 7 mg-15 mg, 7 mg-16 mg, 7 mg-18 mg, 7 mg-20 mg, 7 mg-22 mg, 7 mg-24 mg, 7 mg-26 mg, 7 mg-28 mg, 7 mg-30 mg, 7 mg-32 mg, 7 mg-34 mg, 7 mg-36 mg, 7 mg-38 mg, 7 mg-40 mg, 7 mg-42 mg, 7 mg-44 mg, 7 mg-46 mg, 7 mg-48 mg, 7 mg-50 mg, 7 mg-52 mg, 7 mg-54 mg, 7 mg-56 mg, 7 mg-58 mg, 7 mg-60 mg, 9 mg-10 mg, 9 mg-12 mg, 9 mg-14 mg, 9 mg-15 mg, 9 mg-16 mg, 9 mg-18 mg, 9 mg-20 mg, 9 mg-22 mg, 9 mg-24 mg, 9 mg-26 mg, 9 mg-28 mg, 9 mg-30 mg, 9 mg-32 mg, 9 mg-34 mg, 9 mg-36 mg, 9 mg-38 mg, 9 mg-40 mg, 9 mg-42 mg, 9 mg-44 mg, 9 mg-46 mg, 9 mg-48 mg, 9 mg-50 mg, 9 mg-52 mg, 9 mg-54 mg, 9 mg-56 mg, 9 mg-58 mg, 9 mg-60 mg, 10 mg-12 mg, 10 mg-14 mg, 10 mg-15 mg, 10 mg-16 mg, 10 mg-18 mg, 10 mg-20 mg, 10 mg-22 mg, 10 mg-24 mg, 10 mg-26 mg, 10 mg-28 mg, 10 mg-30 mg, 10 mg-32 mg, 10 mg-34 mg, 10 mg-36 mg, 10 mg-38 mg, 10 mg-40 mg, 10 mg-42 mg, 10 mg-44 mg, 10 mg-46 mg, 10 mg-48 mg, 10 mg-50 mg, 10 mg-52 mg, 10 mg-54 mg, 10 mg-56 mg, 10 mg-58 mg, 10 mg-60 mg, 12 mg-14 mg, 12 mg-15 mg, 12 mg-16 mg, 12 mg-18 mg, 12 mg-20 mg, 12 mg-22 mg, 12 mg-24 mg, 12 mg-26 mg, 12 mg-28 mg, 12 mg-30 mg, 12 mg-32 mg, 12 mg-34 mg, 12 mg-36 mg, 12 mg-38 mg, 12 mg-40 mg, 12 mg-42 mg, 12 mg-44 mg, 12 mg-46 mg, 12 mg-48 mg, 12 mg-50 mg, 12 mg-52 mg, 12 mg-54 mg, 12 mg-56 mg, 12 mg-58 mg, 12 mg-60 mg, 15 mg-16 mg, 15 mg-18 mg, 15 mg-20 mg, 15 mg-22 mg, 15 mg-24 mg, 15 mg-26 mg, 15 mg-28 mg, 15 mg-30 mg, 15 mg-32 mg, 15 mg-34 mg, 15 mg-36 mg, 15 mg-38 mg, 15 mg-40 mg, 15 mg-42 mg, 15 mg-44 mg, 15 mg-46 mg, 15 mg-48 mg, 15 mg-50 mg, 15 mg-52 mg, 15 mg-54 mg, 15 mg-56 mg, 15 mg-58 mg, 15 mg-60 mg, 17 mg-18 mg, 17 mg-20 mg, 17 mg-22 mg, 17 mg-24 mg, 17 mg-26 mg, 17 mg-28 mg, 17 mg-30 mg, 17 mg-32 mg, 17 mg-34 mg, 17 mg-36 mg, 17 mg-38 mg, 17 mg-40 mg, 17 mg-42 mg, 17 mg-44 mg, 17 mg-46 mg, 17 mg-48 mg, 17 mg-50 mg, 17 mg-52 mg, 17 mg-54 mg, 17 mg-56 mg, 17 mg-58 mg, 17 mg-60 mg, 20 mg-22 mg, 20 mg-24 mg, 20 mg-26 mg, 20 mg-28 mg, 20 mg-30 mg, 20 mg-32 mg, 20 mg-34 mg, 20 mg-36 mg, 20 mg-38 mg, 20 mg-40 mg, 20 mg-42 mg, 20 mg-44 mg, 20 mg-46 mg, 20 mg-48 mg, 20 mg-50 mg, 20 mg-52 mg, 20 mg-54 mg, 20 mg-56 mg, 20 mg-58 mg, 20 mg-60 mg, 22 mg-24 mg, 22 mg-26 mg, 22 mg-28 mg, 22 mg-30 mg, 22 mg-32 mg, 22 mg-34 mg, 22 mg-36 mg, 22 mg-38 mg, 22 mg-40 mg, 22 mg-42 mg, 22 mg-44 mg, 22 mg-46 mg, 22 mg-48 mg, 22 mg-50 mg, 22 mg-52 mg, 22 mg-54 mg, 22 mg-56 mg, 22 mg-58 mg, 22 mg-60 mg, 25 mg-26 mg, 25 mg-28 mg, 25 mg-30 mg, 25 mg-32 mg, 25 mg-34 mg, 25 mg-36 mg, 25 mg-38 mg, 25 mg-40 mg, 25 mg-42 mg, 25 mg-44 mg, 25 mg-46 mg, 25 mg-48 mg, 25 mg-50 mg, 25 mg-52 mg, 25 mg-54 mg, 25 mg-56 mg, 25 mg-58 mg, 25 mg-60 mg, 27 mg-28 mg, 27 mg-30 mg, 27 mg-32 mg, 27 mg-34 mg, 27 mg-36 mg, 27 mg-38 mg, 27 mg-40 mg, 27 mg-42 mg, 27 mg-44 mg, 27 mg-46 mg, 27 mg-48 mg, 27 mg-50 mg, 27 mg-52 mg, 27 mg-54 mg, 27 mg-56 mg, 27 mg-58 mg, 27 mg-60 mg, 30 mg-32 mg, 30 mg-34 mg, 30 mg-36 mg, 30 mg-38 mg, 30 mg-40 mg, 30 mg-42 mg, 30 mg-44 mg, 30 mg-46 mg, 30 mg-48 mg, 30 mg-50 mg, 30 mg-52 mg, 30 mg-54 mg, 30 mg-56 mg, 30 mg-58 mg, 30 mg-60 mg, 33 mg-34 mg, 33 mg-36 mg, 33 mg-38 mg, 33 mg-40 mg, 33 mg-42 mg, 33 mg-44 mg, 33 mg-46 mg, 33 mg-48 mg, 33 mg-50 mg, 33 mg-52 mg, 33 mg-54 mg, 33 mg-56 mg, 33 mg-58 mg, 33 mg-60 mg, 36 mg-38 mg, 36 mg-40 mg, 36 mg-42 mg, 36 mg-44 mg, 36 mg-46 mg, 36 mg-48 mg, 36 mg-50 mg, 36 mg-52 mg, 36 mg-54 mg, 36 mg-56 mg, 36 mg-58 mg, 36 mg-60 mg, 40 mg-42 mg, 40 mg-44 mg, 40 mg-46 mg, 40 mg-48 mg, 40 mg-50 mg, 40 mg-52 mg, 40 mg-54 mg, 40 mg-56 mg, 40 mg-58 mg, 40 mg-60 mg, 43 mg-46 mg, 43 mg-48 mg, 43 mg-50 mg, 43 mg-52 mg, 43 mg-54 mg, 43 mg-56 mg, 43 mg-58 mg, 42 mg-60 mg, 45 mg-48 mg, 45 mg-50 mg, 45 mg-52 mg, 45 mg-54 mg, 45 mg-56 mg, 45 mg-58 mg, 45 mg-60 mg, 48 mg-50 mg, 48 mg-52 mg, 48 mg-54 mg, 48 mg-56 mg, 48 mg-58 mg, 48 mg-60 mg, 50 mg-52 mg, 50 mg-54 mg, 50 mg-56 mg, 50 mg-58 mg, 50 mg-60 mg, 52 mg-54 mg, 52 mg-56 mg, 52 mg-58 mg, or 52 mg-60 mg. In some embodiments, the second pharmaceutical agent dose is greater than about 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, or about 200 mg. In some embodiments, the second pharmaceutical agent dose is about less than about 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, or about 200 mg. In some embodiments, the second pharmaceutical agent dose is about 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, about 200 mg, about 225 mg, about 250 mg, about 275 mg, or about 300 mg.

In some embodiments, the mass ratio of TR-β agonist compound to the second pharmaceutical agent is from about 10:1 to about 1:10. In some embodiments, the mass ratio of TR-β agonist compound to the second pharmaceutical agent is from about 7:1 to about 1:7. In some embodiments, the mass ratio of TR-β agonist compound to the second pharmaceutical agent is from about 5:1 to about 1:5. In some embodiments, the mass ratio of TR-β agonist compound to the second pharmaceutical agent is from about 3:1 to about 1:3. In some embodiments, the mass ratio of TR-β agonist compound to the second pharmaceutical agent is from about 2:1 to about 1:2. In some embodiments, the mass ratio of TR-β agonist compound to the second pharmaceutical agent is from about 10:1 to about 1:1, from about 7:1 to about 1:1, from about 5:1 to about 1:1, from about 3:1 to about 1:1, or from about 2:1 to about 1:1. In some embodiments, the mass ratio of TR-β agonist compound to the second pharmaceutical agent is from about 1:1 to about 1:2, from about 1:1 to about 1:3, from about 1:1 to about 1:5, from about 1:1 to about 1:7, or from about 1:1 to about 1:10. In some embodiments, the mass ratio of TR-β agonist compound to the second pharmaceutical agent is about 10:1. 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10, or any range between two of these values.

In some embodiments, the TR-β agonist compound is Compound 2 and the second pharmaceutical agent is

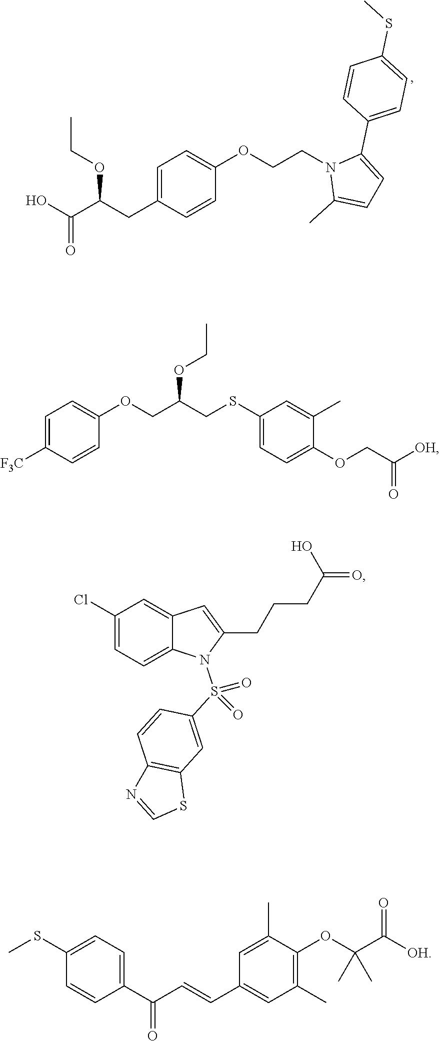 [SMILES: CCO[C@@H](Cc1ccc(OCCn2c(C)ccc2-c2ccc(SC)cc2)cc1)C(=O)O ; CCO[C@H](COc1ccc(C(F)(F)F)cc1)CSc1ccc(OCC(=O)O)c(C)c1 ; O=C(O)CCCc1cc2cc(Cl)ccc2n1S(=O)(=O)c1ccc2ncsc2c1 ; CSc1ccc(C(=O)/C=C/c2cc(C)c(OC(C)(C)C(=O)O)c(C)c2)cc1]

In some embodiments, the TR-β agonist compound is Compound 2 and the second pharmaceutical agent is fenofibrate, gemfibrozil, fenofibric acid, or clofibrate. In some embodiments, the TR-β agonist compound is Compound 2 and the second pharmaceutical agent is

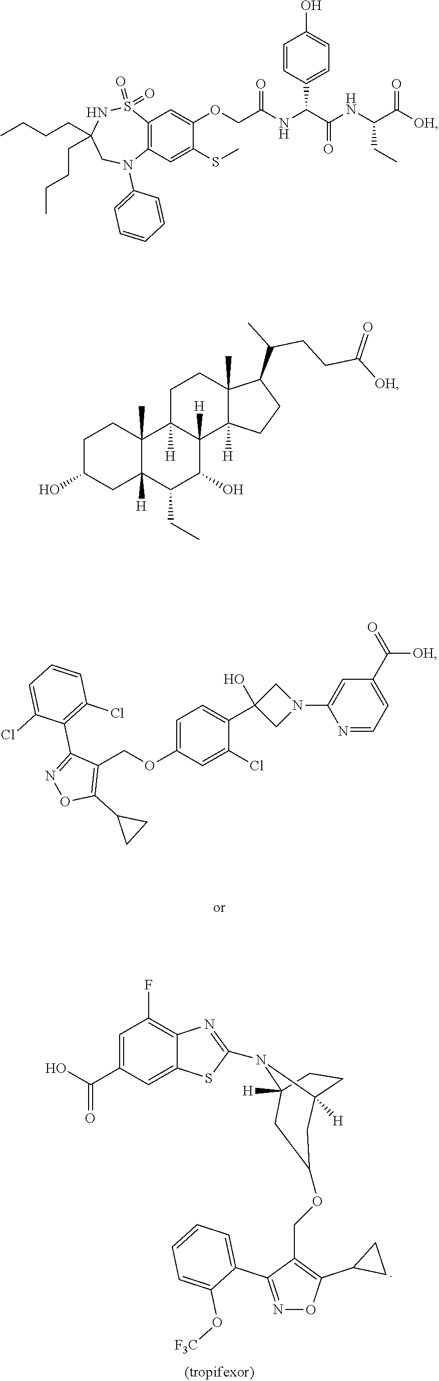

In some embodiments, the TR-β agonist compound is Compound 2 and the second pharmaceutical agent is

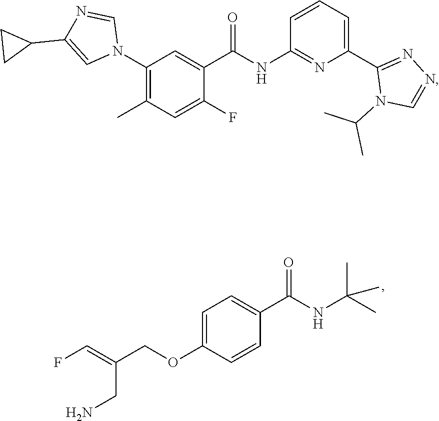 [SMILES: Cc1cc(F)c(C(=O)Nc2cccc(-c3nncn3C(C)C)n2)cc1-n1cnc(C2CC2)c1 ; CC(C)(C)NC(=O)c1ccc(OC/C(=C/F)CN)cc1]

or IMM-124E. In some embodiments, the TR-β agonist compound is Compound 2 and the second pharmaceutical agent is dulaglutide, exenatide, liraglutide, albiglutide, lixisenatide, semaglutide, or insulin glargine. In some embodiments, the TR-β agonist compound is Compound 2 and the second pharmaceutical agent is

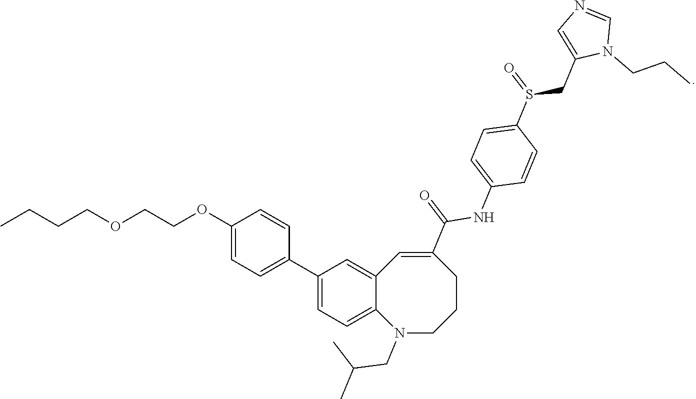 [SMILES: CCCCOCCOc1ccc(-c2ccc3c(c2)/C=C(/C(=O)Nc2ccc([S@@](=O)Cc4cncn4CCC)cc2)CCCN3CC(C)C)cc1]

In some embodiments, the TR-β agonist compound is Compound 2 and the second pharmaceutical agent is

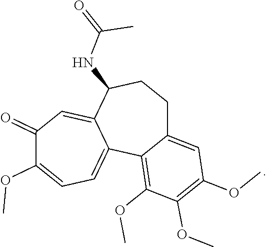

In some embodiments, the TR-β agonist compound is Compound 2 and the second pharmaceutical agent is

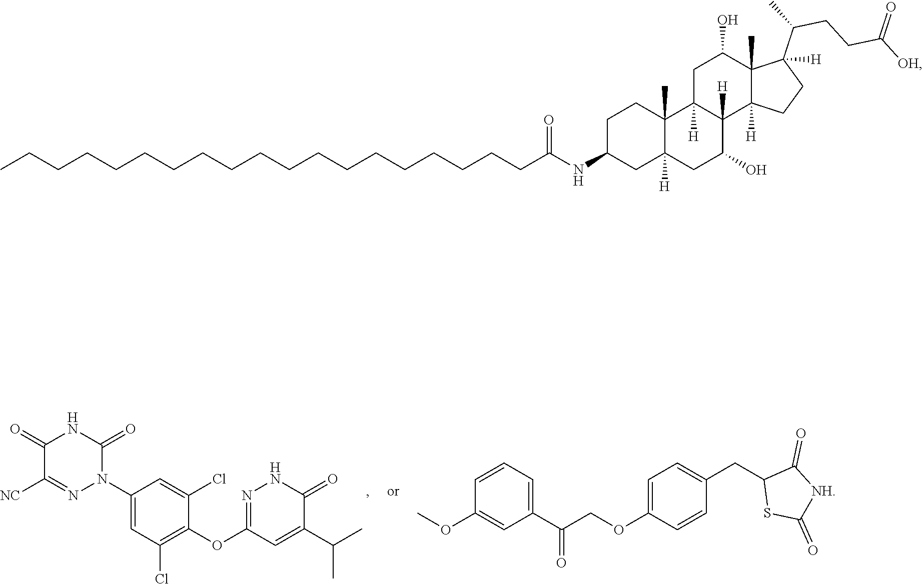

In some embodiments, the TR-β agonist compound is Compound 2 and the second pharmaceutical agent is ethyl (5Z,8Z,11Z,14Z,17Z)-eicosa-5,8,11,14,17-pentaenoate, ethyl (4Z,7Z,10Z,13Z,16Z,19Z)-docosa-4,7,10,13,16,19-hexaenoate, ethyl (7Z,10Z,13Z,16Z,19Z)-docosapentaenoate, ethyl hexadecatrienoate, α-linolenic acid ethyl ester, ethyl (6Z,9Z,12Z,15Z)-6,9,12,15-octadecatetraenoate, ethyl eicosatrienoate, ethyl eicosatetraenoate, ethyl heneicosapentaenoate, ethyl icosapentaenoate, ethyl heneicosapentaenoate, ethyl tetracosapentaenoate, or nisinic acid ethyl ester.

The TR-β agonist compounds described herein and/or the second pharmaceutical agents described herein may also be incorporated into formulations for delivery outside the systemic circulation. Such formulations may include enteric-coated capsules, tablets, soft-gels, spray dried powders, polymer matrices, hydrogels, enteric-coated solids, crystalline solids, amorphous solids, glassy solids, coated micronized particles, liquids, nebulized liquids, aerosols, or microcapsules.

### Methods of Administration

The compositions described above may be administered through any suitable route of administration, for example, by injection, such as subcutaneously, intramuscularly, intraperitoneally, intravenously, or intraarterially; topically, such as by cream, lotion, or patch; orally, such as by a pill, dissolved liquid, oral suspension, buccal film, or mouthrinse; nasally, such as by a nasal aerosol, powder, or spray; or ocularly, such as by an eye drop). In some embodiments, the composition may be administered one, twice, three times, our four times per day. In other embodiments, the composition may be administered once, twice, or three times per week. In other embodiments, the composition is administered every other day, every three days, or every four days. In other embodiments, the composition every other week, every three weeks, or every four weeks. In other embodiments, the composition is administered once per month or twice per month.

In some embodiments, an initial loading dose is administered which is higher than subsequent doses (maintenance doses). The dosage form or mode of administration of a maintenance dose may be different from that used for the loading dose. In any of the embodiments disclosed herein, a maintenance dose may comprise administration of the unit dosage form on any dosing schedule contemplated herein, including but not limited to, monthly or multiple times per month, biweekly or multiple times each two weeks, weekly or multiple times per week, daily or multiple times per day. It is contemplated within the present disclosure that dosing holidays may be incorporated into the dosing period of the maintenance dose. Such dosing holidays may occur immediately after the administration of the loading dose or at any time during the period of administration of the maintenance dose. In some embodiments, the loading dose is 300 mg or less; 250 mg or less, 200 mg or less, 150 mg or less, or 100 mg or less. In some embodiments, the maintenance dose is 300 mg or less; 200 mg or less, 100 mg or less, 50 mg or less, 25 mg or less, 10 mg or less, 5 mg or less, or 1 mg or less.

In some embodiments, the TR-β agonist compounds presented herein may be administered simultaneously with one or more second pharmaceutical agents. In other embodiments, the compounds of the present disclosure may be administered sequentially with one or more second pharmaceutical agents.

In some embodiments, the TR-β agonist compounds may be administered prior to administration of the second pharmaceutical agent. In some embodiments the TR-β agonist compounds may be administered about 15 minutes, about 30 minutes, about 45 minutes, about 1 hour, about 2 hours, about 3 hours, about 4 hours, about 6 hours, about 8 hours, about 12 hours, or about 24 hours prior to administration of a second pharmaceutical agent provided herein. In some embodiments, the TR-β agonist compounds may be administered after administration of the second pharmaceutical agent. In some embodiments the TR-β agonist compounds may be administered about 15 minutes, about 30 minutes, about 45 minutes, about 1 hour, about 2 hours, about 3 hours, about 4 hours, about 6 hours, about 8 hours, about 12 hours, or about 24 hours after administration of a second pharmaceutical agent provided herein.

### Methods of Treatment

Some embodiments according to the methods and compositions of the present disclosure relate to a method for preventing, treating, or ameliorating one or more fatty liver diseases in a subject comprising administering an effective amount of a compound of Formula I described herein in combination with one or more second pharmaceutical agents to a subject in need thereof. In some embodiments, the fatty liver disease may be steatosis. In other embodiments, the fatty liver disease may be non-alcoholic fatty liver disease. In some embodiments, the fatty liver disease may be non-alcoholic steatohepatitis (NASH). In some embodiments, the subject may have two or more of the aforementioned fatty liver diseases.

Some embodiments according to the methods and compositions of the present disclosure relate to a method for the reduction or prevention of the deposition of extracellular matrix proteins, comprising administering an effective amount of a compound of Formula I described herein in combination with one or more second pharmaceutical agents described herein to a subject in need thereof. In some embodiments, said deposition of extracellular matrix proteins may comprise abnormal or excessive deposition of said proteins. In some embodiments, said extracellular matrix proteins may comprise one or more of collagen, keratin, elastin, or fibrin. In some embodiments, said extracellular matrix proteins may comprise collagen. In some embodiments, said extracellular matrix proteins may comprise Type I collagen. In some embodiments, said extracellular matrix proteins may comprise Collagen Type Ia. In some embodiments, said extracellular matrix proteins may comprise Type III collagen. Some embodiments according to the compositions and methods of the present disclosure relate to a method for the treatment of a fibrosis or its symptoms or sequelae, comprising administering an effective amount of a compound described herein to a subject in need thereof.

In some embodiments, the compounds and compositions comprising a compound of Formula I described herein and/or one or more second pharmaceutical agents described herein can be used to treat a variety of conditions arising from fibrosis or inflammation, and specifically including those associated with abnormal collagen deposition. Example conditions include glycogen storage disease type III (GSD III), glycogen storage disease type VI (GSD VI), glycogen storage disease type IX (GSD IX), non-alcoholic steatohepatitis (NASH), cirrhosis, hepatitis, scleroderma, alcoholic fatty liver disease, atherosclerosis, asthma, cardiac fibrosis, organ transplant fibrosis, muscle fibrosis, pancreatic fibrosis, bone-marrow fibrosis, liver fibrosis, cirrhosis of liver and gallbladder, fibrosis of the spleen, pulmonary fibrosis, idiopathic pulmonary fibrosis, diffuse parenchymal lung disease, idiopathic interstitial fibrosis, diffuse interstitial fibrosis, interstitial pneumonitis, desquamative interstitial pneumonia, respiratory bronchiolitis, interstitial lung disease, chronic interstitial lung disease, acute interstitial pneumonitis, hypersensitivity pneumonitis, nonspecific interstitial pneumonia, cryptogenic organizing pneumonia, lymphocytic interstitial pneumonia, pneumoconiosis, silicosis, emphysema, interstitial fibrosis, sarcoidosis, mediastinal fibrosis, cardiac fibrosis, atrial fibrosis, endomyocardial fibrosis, renal fibrosis, chronic kidney disease, Type II diabetes, macular degeneration, keloid lesions, hypertrophic scar, nephrogenic systemic fibrosis, injection fibrosis, complications of surgery, fibrotic chronic allograft vasculopathy and/or chronic rejection in transplanted organs, fibrosis associated with ischemic reperfusion injury, post-vasectomy pain syndrome, fibrosis associated with rheumatoid arthritis, arthrofibrosis, Dupuytren's disease, dermatomyositis-polymyositis, mixed connective tissue disease, fibrous proliferative lesions of the oral cavity, fibrosing intestinal strictures, Crohn's disease, glial scarring, leptomeningeal fibrosis, meningitis, systemic lupus erythematosus, fibrosis due to radiation exposure, fibrosis due to mammary cystic rupture, myelofibrosis, retroperitoneal fibrosis, progressive massive fibrosis, or symptoms or sequelae thereof, or other diseases or conditions resulting in the excessive deposition of extracellular matrix components, such as collagen.

In some embodiments the methods of the present disclosure comprise methods for the treatment, amelioration, or prevention of a fibrotic condition. In some embodiments, said fibrotic condition may be secondary to another condition. In some embodiments, said fibrotic condition or primary condition may further comprise chronic inflammation of an organ, tissue, spatial region, or fluid-connected area of the body of a subject. In some embodiments, said inflammation may comprise activation of one or more TGF-beta dependent signaling pathways. In some embodiments, said TGF-β dependent signaling pathways may comprise one or more elements responsive to T3 or T4. In some embodiments, said fibrotic condition may comprise abnormal or excessive deposition of one or more of collagen, keratin, or elastin. In some embodiments, said fibrotic condition may comprise abnormal or excessive deposition of collagen. In some embodiments, said fibrotic condition may comprise abnormal or excessive deposition of Type I collagen. In some embodiments, said fibrotic condition may comprise abnormal or excessive deposition of Collagen Type Ia. In some embodiments, said fibrotic condition may comprise abnormal or excessive deposition of Type III collagen. In some embodiments said fibrotic condition may comprise one or more of glycogen storage disease type III (GSD III), glycogen storage disease type VI (GSD VI), glycogen storage disease type IX (GSD IX), non-alcoholic steatohepatitis (NASH), cirrhosis, hepatitis, scleroderma, alcoholic fatty liver disease, atherosclerosis, asthma, cardiac fibrosis, organ transplant fibrosis, muscle fibrosis, pancreatic fibrosis, bone-marrow fibrosis, liver fibrosis, cirrhosis of liver and gallbladder, fibrosis of the spleen, scleroderma, pulmonary fibrosis, idiopathic pulmonary fibrosis, diffuse parenchymal lung disease, idiopathic interstitial fibrosis, diffuse interstitial fibrosis, interstitial pneumonitis, desquamative interstitial pneumonia, respiratory bronchiolitis, interstitial lung disease, chronic interstitial lung disease, acute interstitial pneumonitis, hypersensitivity pneumonitis, nonspecific interstitial pneumonia, cryptogenic organizing pneumonia, lymphocytic interstitial pneumonia, pneumoconiosis, silicosis, emphysema, interstitial fibrosis, sarcoidosis, mediastinal fibrosis, cardiac fibrosis, atrial fibrosis, endomyocardial fibrosis, renal fibrosis, chronic kidney disease, Type II diabetes, macular degeneration, keloid lesions, hypertrophic scar, nephrogenic systemic fibrosis, injection fibrosis, complications of surgery, fibrotic chronic allograft vasculopathy and/or chronic rejection in transplanted organs, fibrosis associated with ischemic reperfusion injury, post-vasectomy pain syndrome, fibrosis associated with rheumatoid arthritis, arthrofibrosis, Dupuytren's disease, dermatomyositis-polymyositis, mixed connective tissue disease, fibrous proliferative lesions of the oral cavity, fibrosing intestinal strictures, Crohn's disease, glial scarring, leptomeningeal fibrosis, meningitis, systemic lupus erythematosus, fibrosis due to radiation exposure, fibrosis due to mammary cystic rupture, myelofibrosis, retroperitoneal fibrosis, progressive massive fibrosis. In some embodiments, said fibrotic condition may comprise one or more of GSD III, GSD IX, Non Alcoholic Steatohepatitis, cirrhosis of the liver and/or pancreas, scleroderma, idiopathic pulmonary fibrosis, psoriasis, alcoholic fatty liver disease, Dupuytren's disease, and/or any combination thereof.

According to the methods and compositions of the present disclosure, thyroid receptor agonists such as those disclosed herein, and especially including Compounds 1-4, may be administered in combination with one or more second pharmaceutical agents described herein to a subject for the treatment, amelioration, prevention, or cure of a fibrotic condition, or a condition for which fibrosis is a symptom or sequela. According to the methods and composition as disclosed herein, said fibrotic condition or condition having fibrosis as a sequela may further comprise chronic inflammation. According to the methods and compositions as disclosed herein, said fibrotic condition or condition having fibrosis as a sequela may further comprise activation of one or more TGF-β dependent signaling pathways. According to the methods and compositions as disclosed herein, said fibrotic condition or condition having fibrosis as a sequela may further comprise activation and/or repression of one or more Thyroid Receptor Beta (TRβ) dependent signaling pathways. According to the methods and compositions as disclosed herein, said fibrotic condition or condition having fibrosis as a sequela may further comprise the involvement of signaling pathways responsive to triiodothyronine (T3), thyroxine (T4), any combination thereof, or mimetics thereof. According to the methods and compositions as disclosed herein, said fibrotic condition or condition having fibrosis as a sequela may further comprise the involvement of receptors responsive to T3, T4, any combination thereof, or mimetics thereof. In some embodiments according to the methods and compositions disclosed herein, said fibrotic condition or condition having fibrosis as a sequela may comprise the involvement of TRβ. In some embodiments according to the methods and compositions disclosed herein, said fibrotic condition or condition having fibrosis as a sequela may comprise one or more conditions which are prevented, ameliorated, or cured by the administration of one or more agonists of TRβ. In some embodiments according to the methods and compositions disclosed herein, said fibrotic condition or condition having fibrosis as a sequela may comprise one or more conditions which are prevented, ameliorated, or cured by the administration of one or more of Compounds 1-4 in combination with one or more second pharmaceutical agents described herein. In some embodiments, said one or more agonists of TRβ, or said one or more of Compounds 1-4, and said one or more second pharmaceutical agents, may be co-administered with one or more excipients. In some embodiments, said one or more agonists of TRβ, or said one or more of Compounds 1-4, and said one or more second pharmaceutical agents, may be administered prior to, during, or after a surgical intervention, phototherapy, or ultrasound therapy.

In some embodiments, the compositions and methods described herein provide compositions and methods for the treatment, amelioration, prevention or cure of collagen deposition. In some embodiments, said collagen deposition comprises and abnormal or excessive deposition of collagen. In some embodiments, said collagen deposition may comprise abnormal or excessive deposition of Type I collagen. In some embodiments, said collagen deposition may comprise abnormal or excessive deposition of Collagen Type Ia. In some embodiments, said collagen deposition may comprise abnormal or excessive deposition of Type III collagen. According to the methods and compositions as disclosed herein, said collagen deposition may further comprise the involvement of receptors responsive to T3, T4, any combination thereof, or mimetics thereof. In some embodiments according to the methods and compositions disclosed herein, said collagen deposition may comprise the involvement of TRβ. In some embodiments according to the methods and compositions disclosed herein, said collagen deposition may be prevented, ameliorated, or cured by the administration of one or more agonists of TRβ. In some embodiments according to the methods and compositions disclosed herein, said collagen deposition may be prevented, ameliorated, or cured by the administration of one or more of Compounds 1-4 in combination with one or more second pharmaceutical agents. In some embodiments, said one or more agonists of TRβ, or said one or more of Compounds 1-4, and one or more second pharmaceutical agents, may be coadministered with one or more excipients. In some embodiments, said one or more agonists of TRβ, or said one or more of Compounds 1-4 and one or more second pharmaceutical agents, may be administered prior to, during, or after a surgical intervention, phototherapy, or ultrasound therapy.

In some embodiments, administration of compounds 1-4, of compound 2, or of any of the compounds or compositions as disclosed herein in combination with one or more second pharmaceutical agents described herein results in a reduction in the expression of the Cola1, Col3a1, αSMA, and/or Galectin1 genes or any combination or product thereof in the subject to which said combination is administered. In some embodiments, administration of compounds 1-4, of compound 2, or of any of the compounds or compositions as disclosed herein in combination with one or more second pharmaceutical agents results in a reduction in the degree of fibrosis observable by histology, histochemistry, immunohistochemistry, or the like, and/or reduction s in the amount, accumulation, or distribution of type 1 collagen and/or hydroxyproline or any combination thereof in the subject to which said combination is administered. In some embodiments, administration of compounds 1-4, of compound 2, or of any of the compounds or compositions in combination with one or more second pharmaceutical agents as disclosed herein results in a reduction in total serum lipids, total serum cholesterol, total serum triglycerides, total liver lipids, total liver cholesterol, total liver triglycerides, or any combination thereof.

The methods described herein are further illustrated by the following examples.

### Example 1

DIO-NASH mice were acclimatized for 3 weeks, with pre-treatment liver biopsy samples collected prior to acclimatization. Mice were randomly assigned to one of seven dosing groups, with 12 mice per group. Assigned dosages were: Compound 2 (10 mg/kg); Obeticholic acid (OCA) (30 mg/kg); Cenicriviroc (CVC) (30 mg/kg); Elafibranor (ELA) (30 mg/kg); Compound 2 (10 mg/kg) and Obeticholic acid (30 mg/kg); Compound 2 (10 mg/kg) and Cenicriviroc (30 mg/kg); Compound 2 (10 mg/kg) and Elafibranor (30 mg/kg). One group was mock treated with vehicle only as a control. Dosage forms of the combinations disclosed herein are administered orally once per day. Absolute and relative body weight of the mice were recorded daily. After 8 weeks, animals were sacrificed. Plasma enzymes (P-ALT (alanine aminotransferase) and P-AST (aspartate aminotransferase)), total plasma triglycerides, and total plasma cholesterol were measured, and terminal necropsy of each liver was carried out, determining relative liver weight as a percentage of body weight, assaying total liver biochemistry including total liver triglycerides, and total liver cholesterol, as well as histological evaluation of total liver hydroxyproline, NAFLD activity score (done pre- and post-treatment), fibrosis stage (also done pre- and post-treatment), steatosis, Col1a1 level, and galactin-3 level. Tissue samples were preserved for characterization using RNAseq; RNAseq was used to determine expression levels for genes showing differential expression in compound 2+second pharmaceutical agent-treated vs. vehicle treated animals and/or genes known to be implicated in fibrosis.

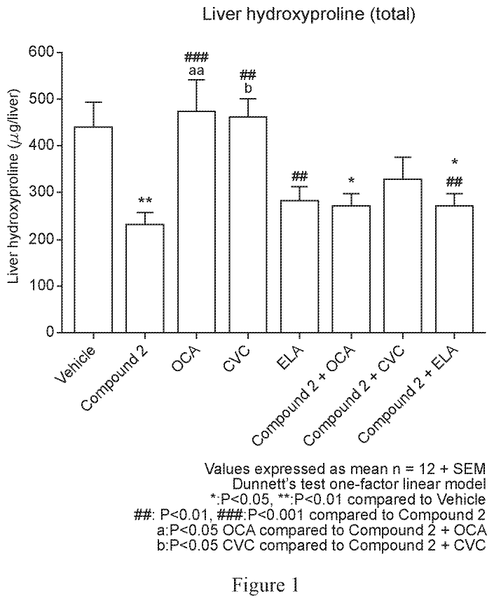

Total liver hyrdoxyproline content is shown in FIG. 1. Since hydroxyproline is a significant component of collagen, and collagen is the most significant source of hydroxyproline in animal tissues, levels of hydroxyproline provide a reliable proxy for the presence of collagen in a sample.

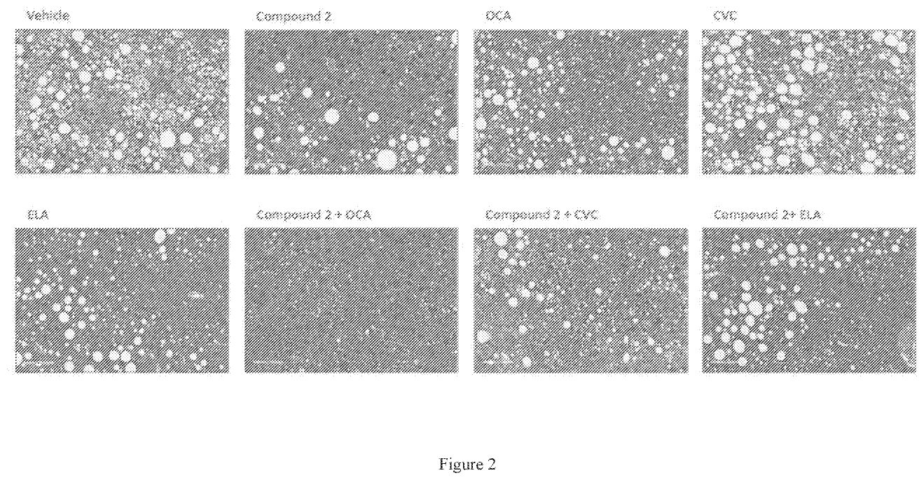

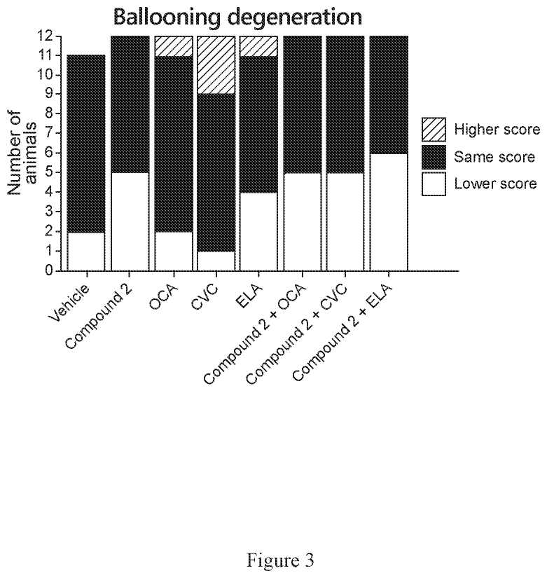

Terminal liver biopsy samples were also subjected to histochemical staining and immunohistochemical staining. Representative images of liver stained with hematoxylin and eosin (HE staining) at the end of the treatment period following 8 weeks of treatment with vehicle, Compound 2, Obeticholic acid, Cenicriviroc, Elafibranor, Compound 2 and Obeticholic acid, Compound 2 and Cenicriviroc, or Compound 2 and Elafibranor are shown in FIG. 2. Ballooning degeneration scores determined were via HE staining of pre-treatment liver biopsy samples and terminal liver biopsy samples (FIG. 3). Fibrosis scores were also calculated based on observation of terminal liver biopsy samples.

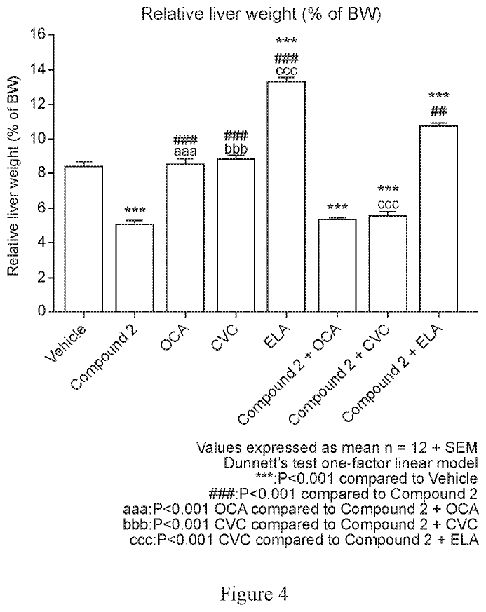

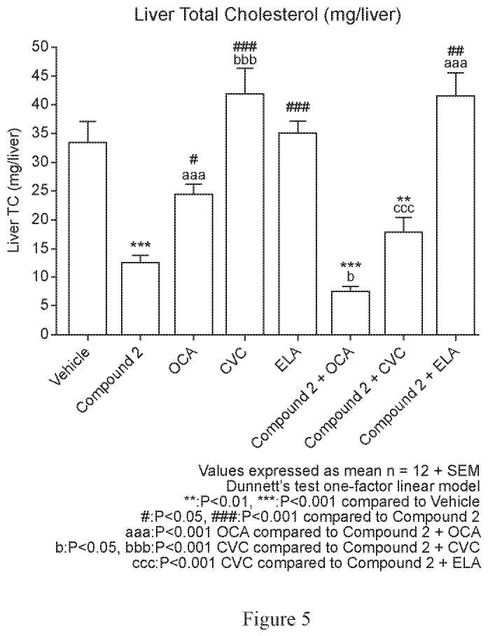

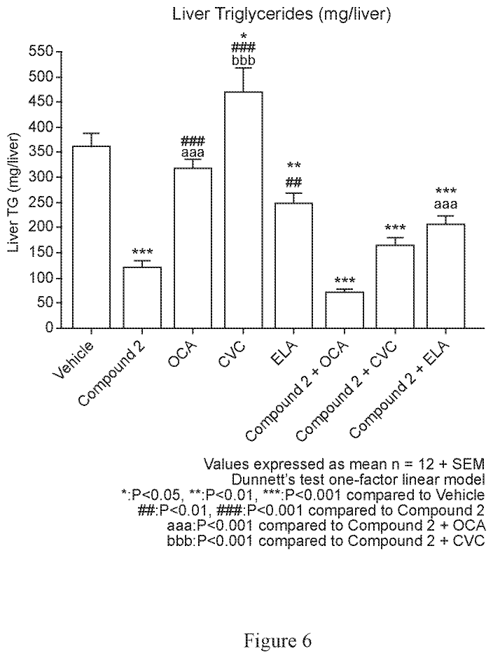

As shown in FIG. 4, the relative liver weight was reduced in compound 2+OCA and compound 2+CVC-treated animals vs. vehicle treated animals. Total liver cholesterol content (FIG. 5) and total liver triglyceride content (FIG. 6) was also reduced in compound 2+OCA and compound 2+CVC-treated animals vs. vehicle treated animals.

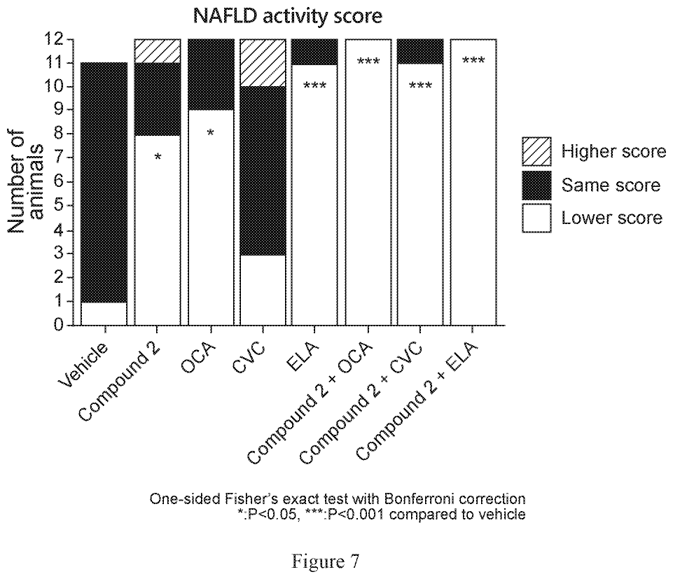

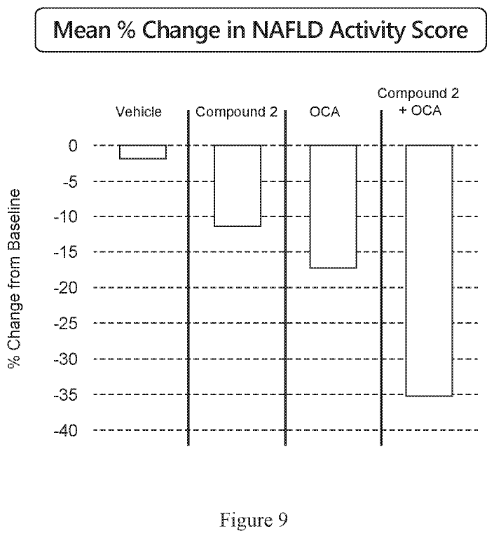

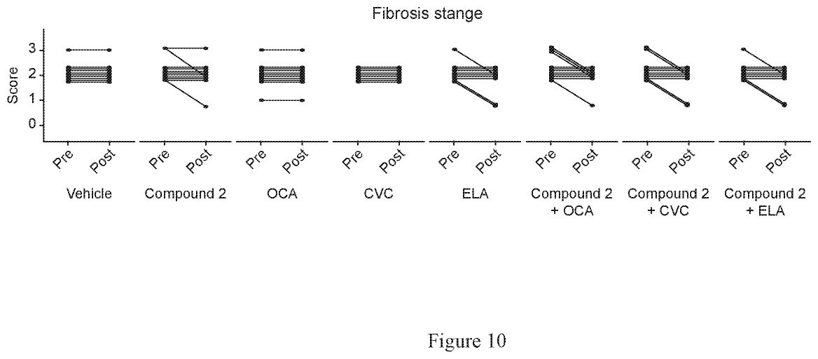

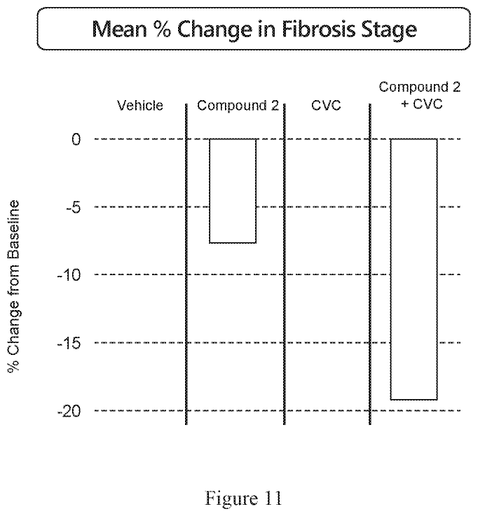

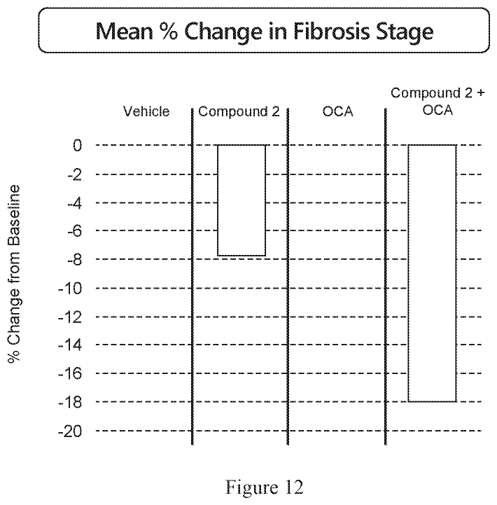

Significant improvements in NAFLD activity scores were observed in compound 2+OCA and compound 2+CVC-treated animals vs. vehicle treated animals or compared to treatment with any of compound 2, CVC, or OCA alone (FIGS. 7-9). Additionally, improvement in fibrosis scores were observed in compound 2+OCA and compound 2+CVC-treated animals vs. either vehicle treated animals or compared to treatment with any of compound 2, CVC, or OCA alone (FIG. 10-12). No significant toxicity was observed. FIGS. 7-9 show that treatment using the combinations of compound 2 and CVC and compound 2 and OCA has a synergistic effect on improving NAFLD activity scores as compared to treatment using any of compound 2, CVC, or OCA alone. Similarly FIGS. 10-12 show that treatment using the combinations of compound 2 and CVC and compound 2 and OCA has a synergistic effect on improving fibrosis scores as compared to treatment using any of compound 2, CVC, or OCA alone.

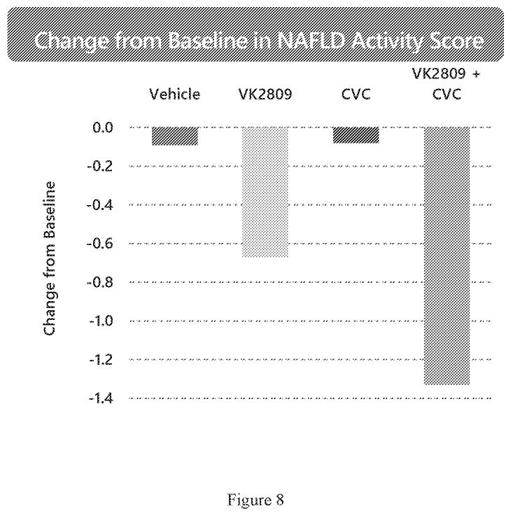

Synergism analysis verified that the combination of Compound 2 and CVC exhibited synergistic effects. The combination of Compound 2 and CVC resulted in larger reductions from baseline in NAFLD activity score compared with the single agents alone (FIG. 8) using a general linear model (p=0.0745). Similarly, analysis of the change from baseline in fibrosis score using a general linear model indicated synergism with a p-value of 0.1967.

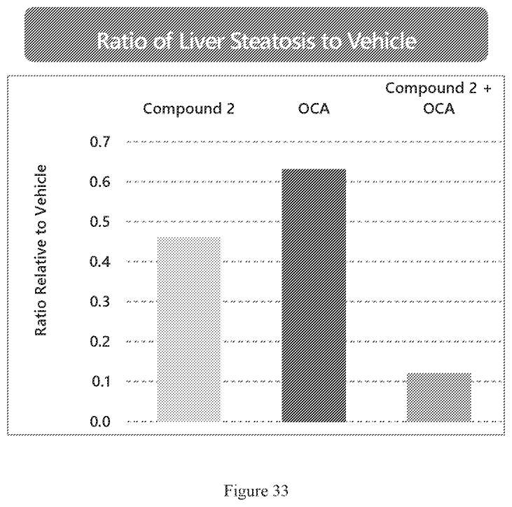

Similarly, Synergism analysis verified that the combination of Compound 2 and OCA exhibited synergistic effects. The ratio of liver steatosis relative to vehicle was analyzed for compound 2 and OCA, alone and in combination (FIG. 33). Analysis of the log-transformed data indicated synergism with a p value of <0.0001.

### Example 2

Palmar fascia fibrosis is induced in nude mice by introducing fibroblasts from fibrotic cords of Dupuytren's disease patients as described in Stish, L. et al., BMC Musculoskelet. Disord. 16: 138-148 (2015) which is hereby incorporated by reference with respect to its description of the establishement of an animal model system for the study of palmar fascia fibrosis. After the establishment of palmar fascia fibrosis in the fibroblast treated animals, test articles comprising any one of Compounds 1-4, or any other compound disclosed herein, are administered to each subject as appropriate for its formulation, daily or as appropriate, in combination with one or second pharmaceutical agents described herein, for 6-10 weeks. Unilateral forepaw biopsies are taken prior to the first administration of the test articles and again after sacrifice following the last administration of the test articles. Biopsy samples are analyzed as described in Example I, with the addition of immunohistochemical staining for type III collagen. Palmar fascia from animals treated with the combination of compound and second pharmaceutical agents disclosed herein show reduced levels of hydroxyproline, decreased Collagen III staining, and decreased fibrosis score relative to the levels shown prior to the administration of the test articles. Mock treated animals show little or no reduction in fibrosis, hydroxyproline content, or Collagen III content.

### Example 3

Hypertrophic skin lesions are induced in Sprague-Dawley Rats by subcutaneous injection of capsaicin as described in Wallengren, J. et al., Skin Pharm. Appl. Skin Physiol. 15(3): 154-165(2002), which is hereby incorporated by reference with respect to its description of the induction of hypertrophic skin lesions in rats; or in C57BL or other appropriate strain mice by subcutaneous administration of CCl₄ and/or bleomycin, as described in Alonso-Merino et al., Proc. Nat. Acad. Sci. 113(24):E3451-60 (2016), which is incorporated herein for its disclosure of the induction of fibrotic skin lesions in mice. After the establishment of hypertrophic skin lesions in the capsaicin, CCl₄ and/or bleomycin treated animals, test articles comprising any one of Compounds 1-4, or any other compound disclosed herein, are administered to each subject animal as appropriate for its formulation, daily or as appropriate, in combination with one or more second pharmaceutical agents described herein, for 6-10 weeks. Skin biopsies from the injection site are taken prior to the first administration of the test articles and again after sacrifice following the last administration of the test articles. Biopsy samples are analyzed as described in Example I, with the addition of immunohistochemical staining for type III collagen. Injection site skin samples from animals treated with the compounds disclosed herein, especially those animals treated with Compound 2 and a second pharmaceutical agent described herein, show reduced levels of hydroxyproline, decreased Collagen III staining, and decreased fibrosis score relative to the levels shown prior to the administration of the test articles. Mock treated animals show little or no reduction in fibrosis, hydroxyproline content, or Collagen III content.

### Example 4

Glucose-6-phosphatase-α deficient mice that manifest GSD-3-like hepatic symptoms, including hypercholesterolemia and hyperlipidemia (Agl−/−, see e.g. Liu, K. M. et al., Mol. Genet. Metabol. 111(4):467-76 (2014)) are treated with test articles comprising any one of Compounds 1-4, or any other compound disclosed herein, administered to each subject as appropriate for its formulation, daily or as appropriate, in combination with one or more second pharmaceutical agents described herein, for 6-10 weeks. Liver biopsies are taken prior to the first administration of the test articles and again after sacrifice following the last administration of the test articles. Biopsy samples are analyzed as described in Example I. Liver samples from animals treated with the compounds and second pharmaceutical agents disclosed herein show reduced levels of hydroxyproline, decreased Collagen I staining, and decreased fibrosis score relative to the levels shown prior to the administration of the test articles. Mock treated animals show little or no reduction in fibrosis, hydroxyproline content, or Collagen I content.

### Example 5

Phosphorylase kinase deficient mice that manifest GSD-8/9-like hepatic symptoms, including hypercholesterolemia and hyperlipidemia (PhKc−/−, see, e.g., Varsanyi, M. et al., Biochem. Genet. 18(3-4):247-61 (1980)), are treated with test articles comprising any one of Compounds 1-4, or any other compound disclosed herein, administered to each subject as appropriate for its formulation, daily or as appropriate, in combination with one or more second pharmaceutical agents described herein, for 6-10 weeks. Liver biopsies are taken prior to the first administration of the test articles and again after sacrifice following the last administration of the test articles. Biopsy samples are analyzed as described in Example I. Liver samples from animals treated with the combinations disclosed herein, especially those animals treated with Compound 2 and a second pharmaceutical agent, show reduced levels of hydroxyproline, decreased Collagen I staining, and decreased fibrosis score relative to the levels shown prior to the administration of the test articles. Mock treated animals show little or no reduction in fibrosis, hydroxyproline content, or Collagen I content.

### Example 6

Following GAN diet-induction (GAN=Gubra Amylin NASH diet, a AMLN diet with Primex substituted by plam oil), DIO-NASH mice were randomly assigned to one of six dosing groups, with 13-15 mice per group. Assigned dosages were: Vehicle (administered PO, BID) [n=15]; Compound 2 (10 mg/kg administered PO, QD) [n=13]; semaglutide (30 nmol/kg administered SC, QD)[n=15]; Compound 2 (10 mg/kg administered PO, QD) and semaglutide (30 mg/kg administered SC, QD) [n=15]; tropifexor (0.3 mg/kg administered PO, QD) [n=14]; and Compound 2 (10 mg/kg administered PO, QD) and tropifexor (30 mg/kg administered PO, QD) [n=15]. The mice undergo a 14 day titration period with a total dosing period of 12 weeks. Baseline steatosis score, fibrosis stage, and Col1a1 levels are determined one week prior to dosing. Body weight of the mice were recorded daily. After 12 weeks of dosing, animals were sacrificed. Plasma enzymes P-ALT (alanine aminotransferase) and P-AST (aspartate aminotransferase)), total plasma triglycerides, and total plasma cholesterol were measured, and terminal necropsy of each liver was carried out, determining relative liver weight as a percentage of body weight, assaying total liver biochemistry including total liver triglycerides, and total liver cholesterol, as well as histological evaluation of total liver hydroxyproline, NAFLD activity score (done pre- and post-treatment), fibrosis stage (also done pre- and post-treatment), steatosis, Col1a1 level, α-SMA, and galactin-3 level. Tissue samples were preserved for characterization using RNAseq; RNAseq was used to determine expression levels for genes showing differential expression in compound 2+second pharmaceutical agent-treated vs. vehicle treated animals and/or genes known to be implicated in fibrosis.

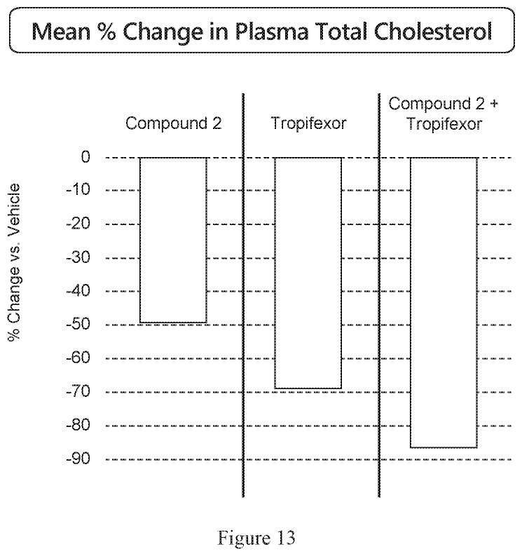

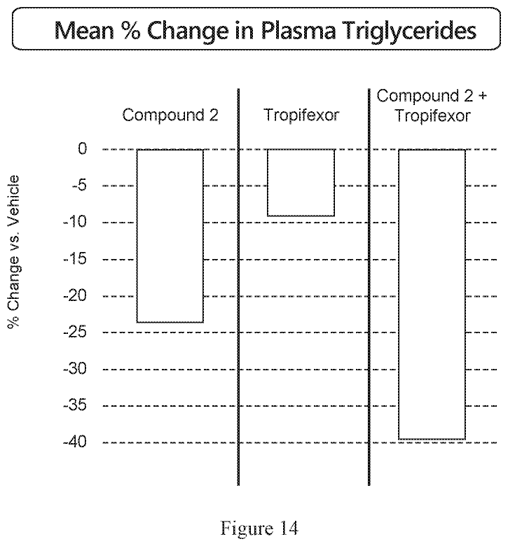

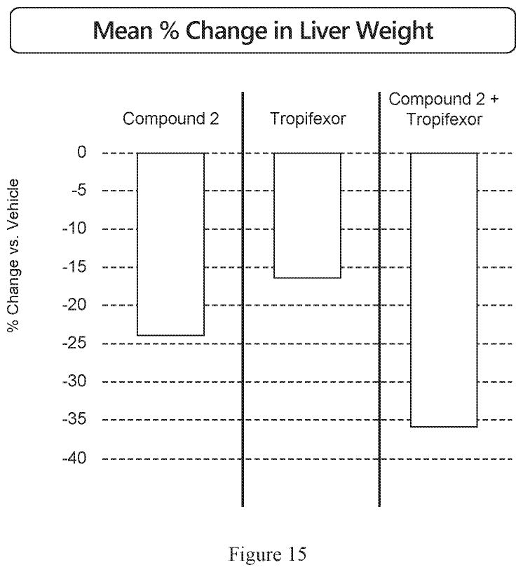

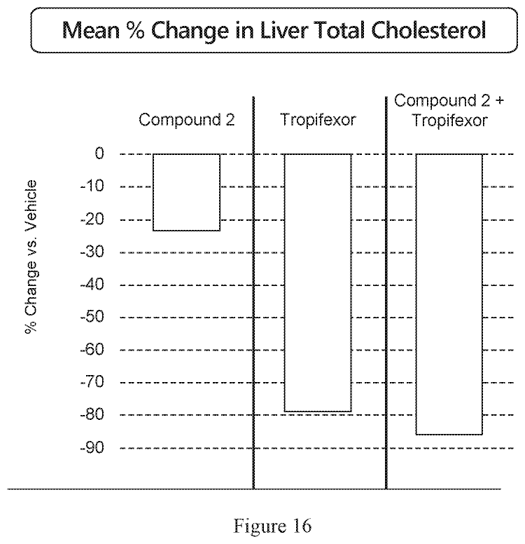

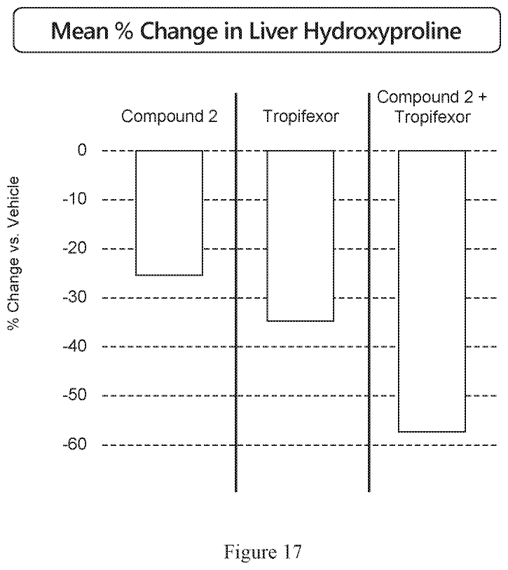

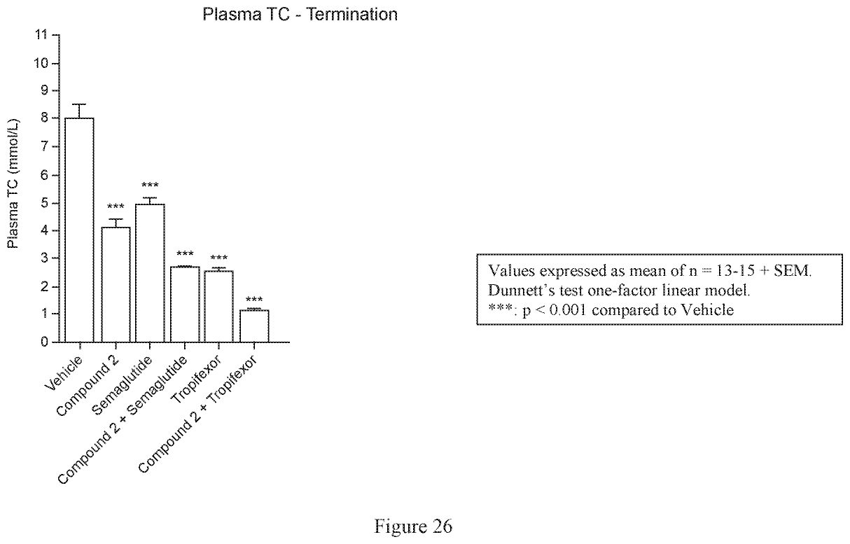

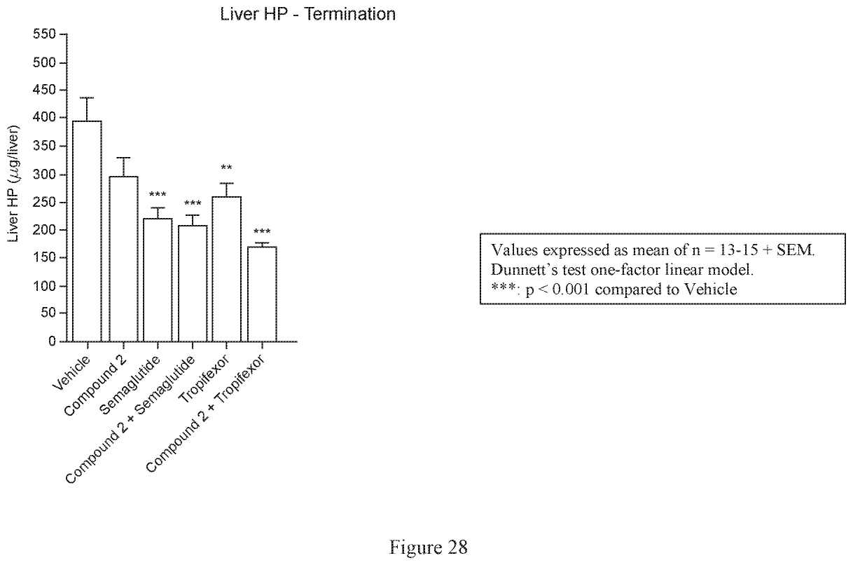

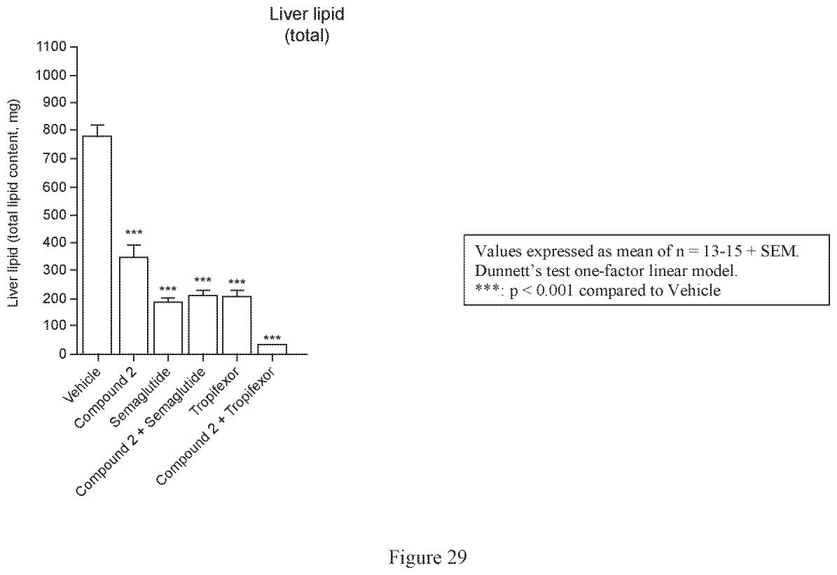

The combination of Compound 2 and tropifexor reduced plasma total cholesterol in treated animals relative to vehicle more than either Compound 2 or tropifexor alone (FIGS. 13 and 26). P-values of the combination of Compound 2 and tropifexor were less than 0.001 when compared versus vehicle or versus either of Compound 2 or tropifexor. The combination of Compound 2 and tropifexor was also effective in reducing plasma triglycerides (FIG. 14) (p-values of the combination of Compound 2 and tropifexor were less than 0.05 when compared versus vehicle or versus either of Compound 2 or tropifexor), and reduing liver weight (FIG. 15) in the treated animals. Additionally, the combination of Compound 2 and tropifexor was effective in reducing liver total cholesterol (FIG. 16) (p-values of the combination of Compound 2 and tropifexor were less than 0.05 when compared versus vehicle or versus either of Compound 2 or tropifexor), reducing liver hydroxyproline (FIGS. 17 and 28) (p-values of the combination of Compound 2 and tropifexor were less than 0.01 when compared versus vehicle or versus either of Compound 2 or tropifexor); and reducing total liver lipids (FIG. 29) (p-values of the combination of Compound 2 and tropifexor were less than 0.001 when compared versus vehicle or versus either of Compound 2 or tropifexor).

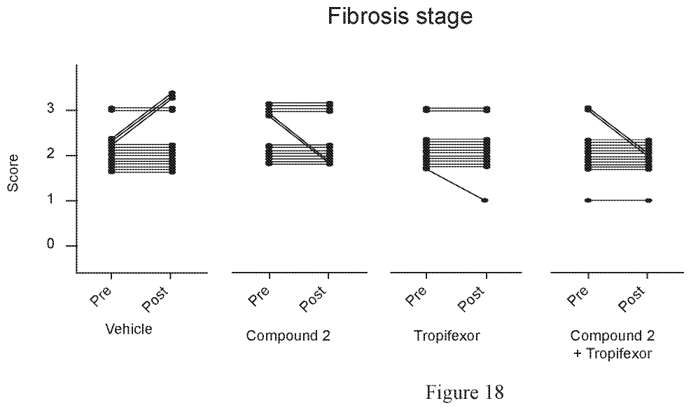

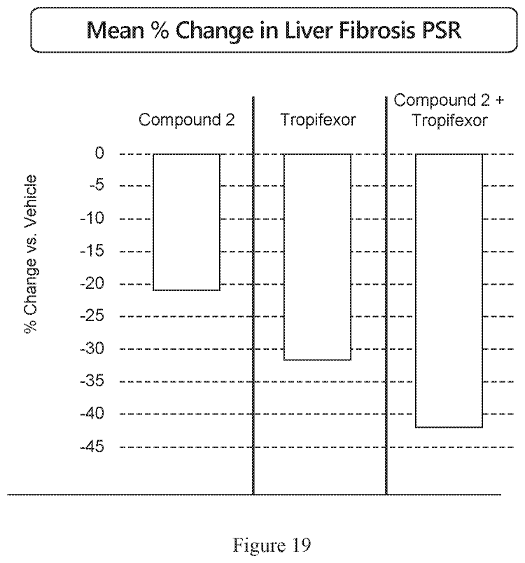

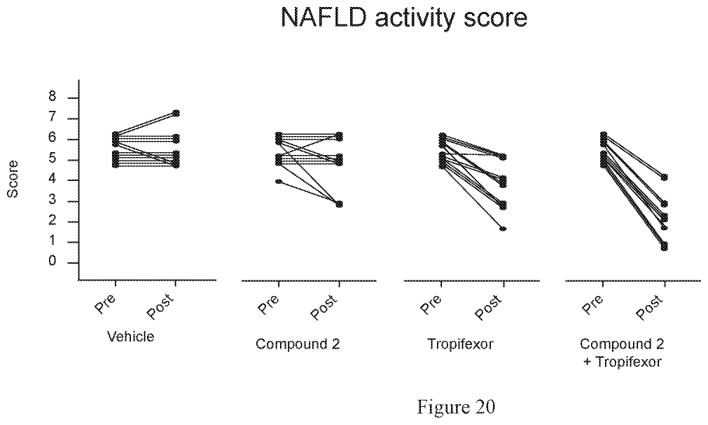

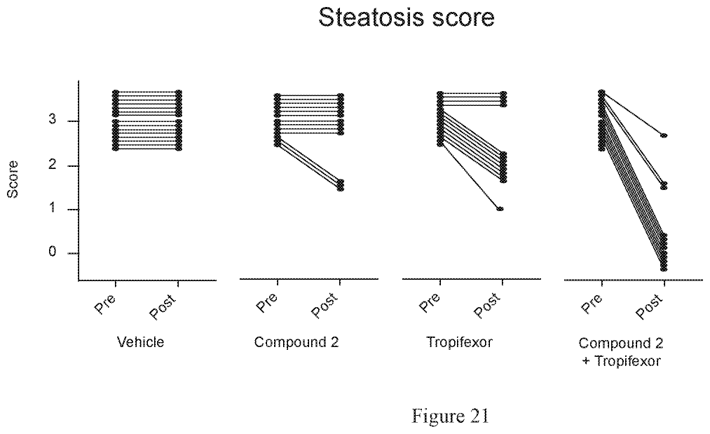

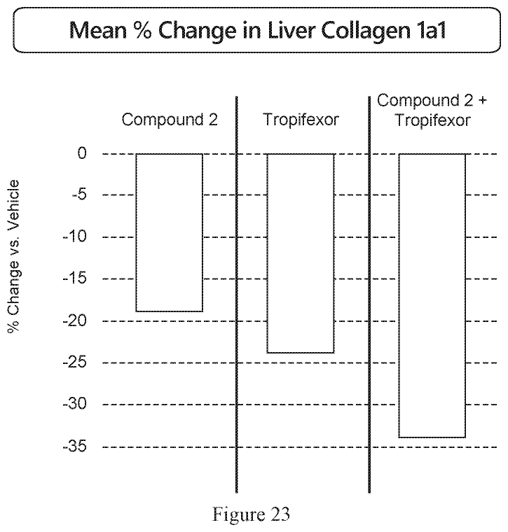

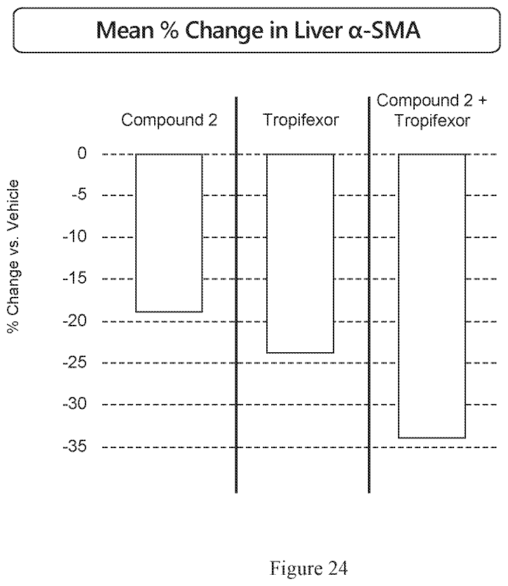

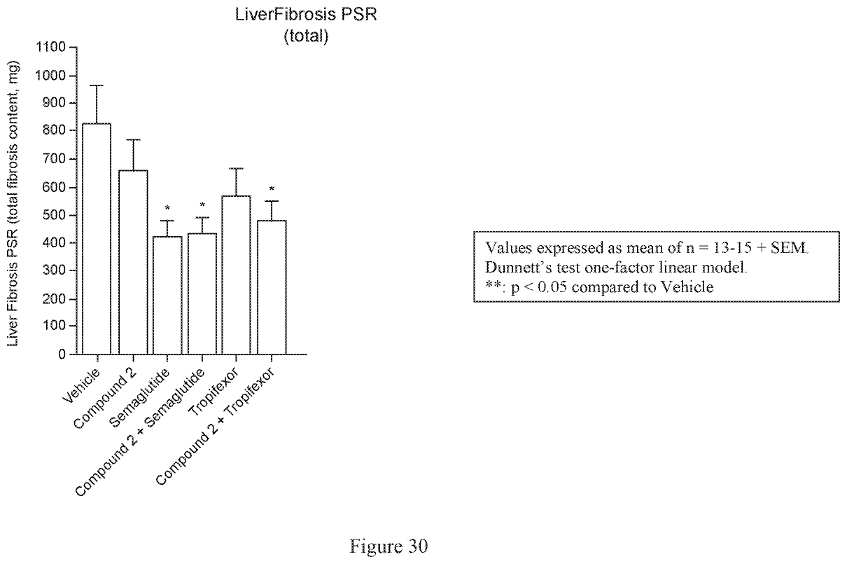

Improvement in fibrosis scores were observed in compound 2+tropifexor-treated animals vs. vehicle treated animals (FIG. 18), while a decrease in liver fibrosis was observed for in compound 2+tropifexor-treated animals vs. vehicle treated animals and animals treated with Compound 2 or tropifexor alone (FIGS. 19 and 30). Additionally, improvements in NAFLD activity score and steatosis scores were observed in compound 2+tropifexor-treated animals vs. vehicle treated animals or compared to treatment with any of compound 2 or tropifexor alone (FIGS. 20-21). Similarly FIGS. 23-24 show that treatment using the combination of compound 2 and tropifexor is effective in reducing liver collagen 1a1 (p-values of the combination of Compound 2 and tropifexor were less than 0.05 when compared versus vehicle or versus either of Compound 2 or tropifexor), and liver α-SMA levels.

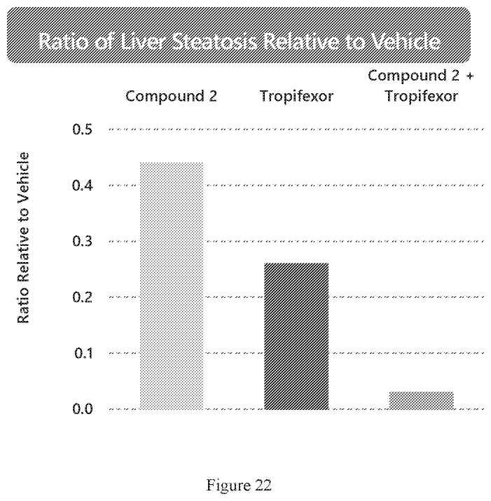

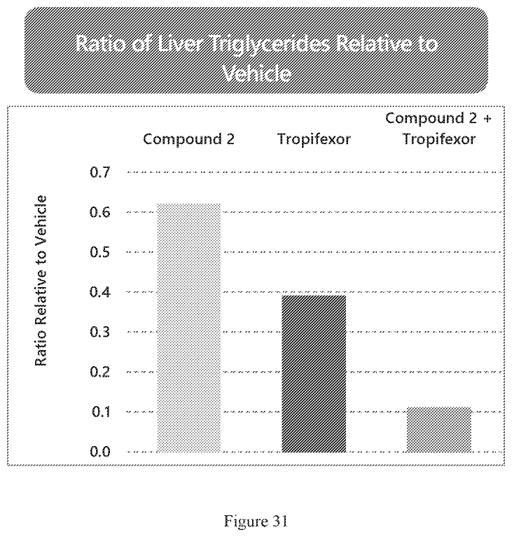

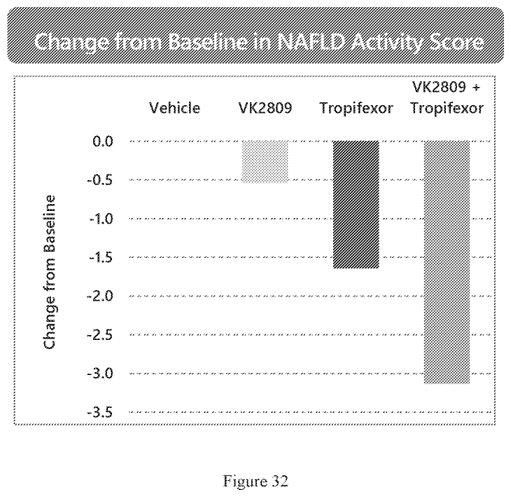

Synergism analysis demonstrated that the combination of Compound 2 and tropifexor exhibited synergistic effects. The ratio of liver steatosis relative to vehicle was determined for the agents alone and in combination (FIG. 22). Analysis of the log-transformed data indicated synergism with a p-value of 0.0004. Similarly, the ratio of liver triglycerides relative to vehicle was determined for the agents alone and in combination (FIG. 31). The same analyses of the log-transformed data also indicted synergism with a p-value of 0.0018. Finally, analysis of the change from baseline in NALFD activity score for the agents alone and in combination (FIG. 32) using a general linear model indicated synergism with a p-value of 0.0297.

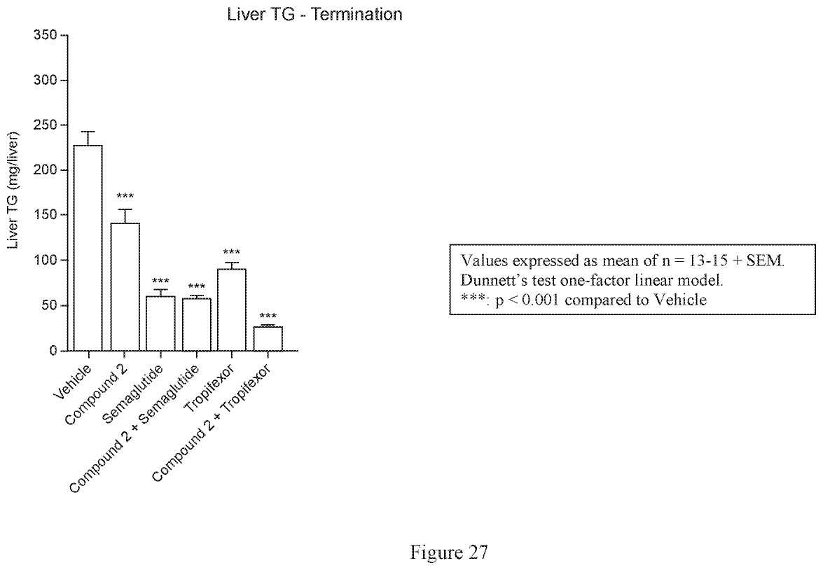

The combination of Compound 2 and semaglutide reduced plasma total cholesterol in treated animals relative to vehicle more than either Compound 2 or semaglutide alone (FIG. 26). The combination of Compound 2 and semaglutide was also effective in reducing liver triglycerides (FIG. 27) in the treated animals. Additionally, the combination of Compound 2 and semaglutide was effective in reducing liver total cholesterol (FIG. 16), liver hydroxyproline, (FIG. 28) and total liver lipids (FIG. 29). A decrease in liver fibrosis was also observed for in compound 2+semaglutide-treated animals vs. vehicle treated animals.

### Example 7

DIO-NASH mice are randomly assigned to a dosing group selected from vehicle, Compound 2 (e.g., 10 mg/kg), liraglutide (e.g., 0.2 mg/kg), or Compound 2 (e.g., 10 mg/kg) and liraglutide (e.g., 0.2 mg/kg). Absolute and relative body weight of the mice are recorded daily. The mice undergo a 14 day titration period with a total dosing period of 12 weeks. Baseline steatosis score, fibrosis stage, and Col1a1 levels are determined one week prior to dosing. Body weight of the mice are recorded daily. After 12 weeks of dosing, animals are sacrificed. Plasma enzymes P-ALT (alanine aminotransferase) and P-AST (aspartate aminotransferase)), total plasma triglycerides, and total plasma cholesterol are measured, and terminal necropsy of each liver is carried out, determining relative liver weight as a percentage of body weight, assaying total liver biochemistry including total liver triglycerides, and total liver cholesterol, as well as histological evaluation of total liver hydroxyproline, NAFLD activity score (done pre- and post-treatment), fibrosis stage (also done pre- and post-treatment), steatosis, Col1a1 level, α-SMA, and galactin-3 level. Tissue samples are preserved for characterization using RNAseq; RNAseq is used to determine expression levels for genes showing differential expression in compound 2+liraglutide-treated vs. vehicle treated animals and/or genes known to be implicated in fibrosis.

### Example 8

DIO-NASH mice are randomly assigned to a dosing group selected from vehicle, Compound 2 (e.g., 10 mg/kg), a dual acting GLP-1/glucagon agonist, or Compound 2 (e.g., 10 mg/kg) and the dual acting GLP-1/glucagon agonist. Absolute and relative body weight of the mice are recorded daily. The mice undergo a 14 day titration period with a total dosing period of 12 weeks. Baseline steatosis score, fibrosis stage, and Col1a1 levels are determined one week prior to dosing. Body weight of the mice are recorded daily. After 12 weeks of dosing, animals are sacrificed. Plasma enzymes P-ALT (alanine aminotransferase) and P-AST (aspartate aminotransferase)), total plasma triglycerides, and total plasma cholesterol are measured, and terminal necropsy of each liver is carried out, determining relative liver weight as a percentage of body weight, assaying total liver biochemistry including total liver triglycerides, and total liver cholesterol, as well as histological evaluation of total liver hydroxyproline, NAFLD activity score (done pre- and post-treatment), fibrosis stage (also done pre- and post-treatment), steatosis, Col1a1 level, α-SMA, and galactin-3 level. Tissue samples are preserved for characterization using RNAseq; RNAseq is used to determine expression levels for genes showing differential expression in compound 2+dual acting GLP-1/glucagon agonist-treated vs. vehicle treated animals and/or genes known to be implicated in fibrosis.

### Example 9

DIO-NASH mice are randomly assigned to a dosing group selected from vehicle, Compound 2 (e.g., 10 mg/kg), dual acting GLP-1/GIP agonist, or Compound 2 (e.g., 10 mg/kg) and dual acting GLP-1/GIP agonist. Absolute and relative body weight of the mice are recorded daily. The mice undergo a 14 day titration period with a total dosing period of 12 weeks. Baseline steatosis score, fibrosis stage, and Col1a1 levels are determined one week prior to dosing. Body weight of the mice are recorded daily. After 12 weeks of dosing, animals are sacrificed. Plasma enzymes P-ALT (alanine aminotransferase) and P-AST (aspartate aminotransferase)), total plasma triglycerides, and total plasma cholesterol are measured, and terminal necropsy of each liver is carried out, determining relative liver weight as a percentage of body weight, assaying total liver biochemistry including total liver triglycerides, and total liver cholesterol, as well as histological evaluation of total liver hydroxyproline, NAFLD activity score (done pre- and post-treatment), fibrosis stage (also done pre- and post-treatment), steatosis, Col1a1 level, α-SMA, and galactin-3 level. Tissue samples are preserved for characterization using RNAseq; RNAseq is used to determine expression levels for genes showing differential expression in compound 2+dual acting GLP-1/GIP agonist-treated vs. vehicle treated animals and/or genes known to be implicated in fibrosis.

### Example 10

DIO-NASH mice are randomly assigned to a dosing group selected from vehicle, Compound 2 (e.g., 10 mg/kg), a DGAT inhibitor, or Compound 2 (e.g., 10 mg/kg) and DGAT inhibitor. Absolute and relative body weight of the mice are recorded daily. The mice undergo a 14 day titration period with a total dosing period of 12 weeks. Baseline steatosis score, fibrosis stage, and Col1a1 levels are determined one week prior to dosing. Body weight of the mice are recorded daily. After 12 weeks of dosing, animals are sacrificed. Plasma enzymes P-ALT (alanine aminotransferase) and P-AST (aspartate aminotransferase)), total plasma triglycerides, and total plasma cholesterol are measured, and terminal necropsy of each liver is carried out, determining relative liver weight as a percentage of body weight, assaying total liver biochemistry including total liver triglycerides, and total liver cholesterol, as well as histological evaluation of total liver hydroxyproline, NAFLD activity score (done pre- and post-treatment), fibrosis stage (also done pre- and post-treatment), steatosis, Col1a1 level, α-SMA, and galactin-3 level. Tissue samples are preserved for characterization using RNAseq; RNAseq is used to determine expression levels for genes showing differential expression in compound 2+DGAT inhibitor-treated vs. vehicle treated animals and/or genes known to be implicated in fibrosis

With respect to the use of substantially any plural and/or singular terms herein, those having skill in the art can translate from the plural to the singular and/or from the singular to plural as is appropriate to the context and/or application. The various singular/plural permutations can be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims can contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method of preventing, treating, or ameliorating one or more fatty liver diseases in a subject in need thereof comprising administering to said subject in need thereof at least one TR-β agonist in combination with one or more second pharmaceutical agents.

2. The method of claim 1, wherein the TR-β agonist is a compound of Formula I: 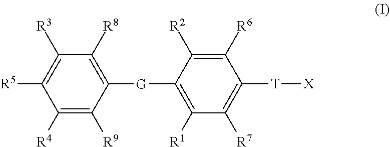
or a pharmaceutically acceptable salt thereof, wherein:
G is selected from the group consisting of —O—, —S—, —S(═O)—, —S(═O)₂—, —Se—, —CH₂—, —CF₂—, —CHF—, —C(O)—, —CH(OH)—, —CH(C₁-C₄ alkyl)-, —CH(C₁-C₄ alkoxy)-, —C(═CH₂)—, —NH—, and —N(C₁-C₄ alkyl)-;
T is selected from the group consisting of —(CR^{a}₂)ₖ—, —CR^{b}═CR^{b}—(CR^{a}₂)ₙ—, —(CR^{a}₂)ₙ—CR^{b}═CR^{b}—, —(CR^{a}₂)—CR^{b}═CR^{b}—(CR^{a}₂)—, —O(CR^{b}₂)(CR^{a}₂)ₙ—, —S(CR^{b}₂)(CR^{a}₂)ₙ—, N(R^{c})(CR^{b}₂)(CR^{a}₂)ₙ—, N(R^{b})C(O)(CR^{a}₂)ₙ, —C(O)(CR^{a}₂)ₘ—, —(CR^{a}₂)ₘC(O)—, —(CR^{a}₂)C(O)(CR^{a}₂)ₙ, —(CR^{a}₂)ₙC(O)(CR^{a}₂)—, and —C(O)NH(CR^{b}₂)(CR^{a}₂)_{P}—;
k is an integer from 1-4;
m is an integer from 0-3;
n is an integer from 0-2;
p is an integer from 0-1;
each R^{a} is independently selected from the group consisting of hydrogen, optionally substituted —C₁-C₄ alkyl, halogen, —OH, optionally substituted —O—C₁-C₄ alkyl, —OCF₃, optionally substituted —S—C₁-C₄ alkyl, —NR^{b}R^{c}, optionally substituted —C₂-C₄ alkenyl, and optionally substituted —C₂-C₄ alkynyl; with the proviso that when one R^{a} is attached to C through an O, S, or N atom, then the other R^{a} attached to the same C is a hydrogen, or attached via a carbon atom;
each R^{b} is independently selected from the group consisting of hydrogen and optionally substituted —C₁-C₄ alkyl;
each R^{c} is independently selected from the group consisting of hydrogen and optionally substituted —C₁-C₄ alkyl, optionally substituted —C(O)—C₁-C₄ alkyl, and —C(O)H;
R¹, and R² are each independently selected from the group consisting of halogen, optionally substituted —C₁-C₄ alkyl, optionally substituted —S—C₁-C₃ alkyl, optionally substituted —C₂-C₄ alkenyl, optionally substituted —C₂-C₄ alkynyl, —CF₃, —OCF₃, optionally substituted-O—C₁-C₃ alkyl, and cyano;
R⁶, R⁷, R⁸, and R⁹ are each independently selected from the group consisting of are each independently selected from the group consisting of hydrogen, halogen, optionally substituted —C C₁-C₄ alkyl, optionally substituted —S—C₁-C₃ alkyl, optionally substituted —C₂-C₄ alkenyl, optionally substituted —C₂-C₄ alkynyl, —CF₃, —OCF₃, optionally substituted-O—C₁-C₃ alkyl, and cyano; or R⁶ and T are taken together along with the carbons they are attached to form a ring of 5 to 6 atoms including 0 to 2 heteroatoms independently selected from —NR¹—, —O—, and —S—, with the proviso that when there are 2 heteroatoms in the ring and both heteroatoms are different than nitrogen then both heteroatoms have to be separated by at least one carbon atom; and X is attached to this ring by a direct bond to a ring carbon, or by —(CR^{a}₂)— or —C(O)— bonded to a ring carbon or a ring nitrogen;
Rⁱ is selected from the group consisting of hydrogen, —C(O)C₁-C₄ alkyl, —C₁-C₄ alkyl, and —C₁-C₄-aryl;
R³ and R⁴ are independently selected from the group consisting of hydrogen, halogen, —CF₃, —OCF₃, cyano, optionally substituted —C₁-C₁₂ alkyl, optionally substituted —C₂-C₁₂ alkenyl, optionally substituted —C₂-C₁₂ alkynyl, —SR^{d}, —S(═O)R^{e}, —S(═O)₂R^{e}, —S(═O)₂NR^{f}R^{g}, —C(O)OR^{h}, —C(O)R^{e}, —N(R^{b})C(O)NR^{f}R^{g}, —N(R^{b})S(═O)₂R^{e}, —N(R^{b})S(═O)₂NR^{f}R^{g}, and —NR^{f}R^{g};
each R^{d} is selected from the group consisting of optionally substituted —C₁-C₁₂ alkyl, optionally substituted —C₂-C₁₂ alkenyl, optionally substituted —C₂-C₁₂ alkynyl, optionally substituted —(CR^{b}₂)ₙ aryl, optionally substituted —(CR^{b}₂)ₙ cycloalkyl, optionally substituted —(CR^{b}₂)ₙ heterocycloalkyl, and —C(O)NR^{f}R^{g};
each R^{e} is selected from the group consisting of optionally substituted —C₁-C₁₂ alkyl, optionally substituted —C₂-C₁₂ alkenyl, optionally substituted —C₂-C₁₂ alkynyl, optionally substituted —(CR^{a}₂)ₙ aryl, optionally substituted —(CR^{a}₂)ₙ cycloalkyl, and optionally substituted —(CR^{a}₂)ₙ heterocycloalkyl;
R^{f} and R^{g} are each independently selected from the group consisting of hydrogen, optionally substituted —C₁-C₁₂ alkyl, optionally substituted —C₂-C₁₂ alkenyl, optionally substituted —C₂-C₁₂ alkynyl, optionally substituted —(CR^{b}₂)ₙ aryl, optionally substituted —(CR^{b}₂)ₙ cycloalkyl, and optionally substituted —(CR^{b}₂)ₙ heterocycloalkyl, or R^{f} and R^{g} may together form an optionally substituted heterocyclic ring, which may contain a second heterogroup selected from the group consisting of O, NR^{C}, and S, wherein said optionally substituted heterocyclic ring may be substituted with 0-4 substituents selected from the group consisting of optionally substituted —C₁-C₄ alkyl, —OR^{b}, oxo, cyano, —CF₃, optionally substituted phenyl, and —C(O)OR^{h};
each R^{h} is selected from the group consisting of optionally substituted —C₁-C₁₂ alkyl, optionally substituted —C₂-C₁₂ alkenyl, optionally substituted —C₂-C₁₂ alkynyl, optionally substituted —(CR^{b}₂)ₙ aryl, optionally substituted —(CR^{b}₂)ₙ cycloalkyl, and optionally substituted —(CR^{b}₂)ₙ heterocycloalkyl;
R⁵ is selected from the group consisting of —OH, optionally substituted —OC₁-C₆, alkyl, OC(O)R^{e}, —OC(O)OR^{h}, —F, —NHC(O)R^{e}, —NHS(═O)R^{e}, —NHS(═O)₂R^{e}, —NHC(═S)NH(R^{h}), and —NHC(O)NH(R^{h});
X is P(O)YR¹¹Y′R¹¹;
Y and Y′ are each independently selected from the group consisting of —O—, and —NR^{v}—; when Y and Y′ are —O—, R¹¹ attached to —O— is independently selected from the group consisting of —H, alkyl, optionally substituted aryl, optionally substituted heterocycloalkyl, optionally substituted CH₂-heterocycloakyl wherein the cyclic moiety contains a carbonate or thiocarbonate, optionally substituted -alkylaryl, —C(R^{z})₂OC(O)NR^{z}₂, —NR^{z}—C(O)—R^{y}, —C(R^{z})₂—OC(O)R^{y}, —C(R^{z})₂—O—C(O)OR^{y}, —C(R^{z})₂OC(O)SR^{y}, -alkyl-S—C(O)R^{y}, -alkyl-S—S-alkylhydroxy, and -alkyl-S—S—S-alkylhydroxy;
when Y and Y′ are —NR^{v}—, then R¹¹ attached to —NR^{v}— is independently selected from the group consisting of —H, —[C(R^{z})₂]_{q}—COOR^{y}, —C(R^{x})₂COOR^{Y}, —[C(R^{z})₂]_{q}—C(O)SR^{y}, and -cycloalkylene-COOR^{y};
when Y is —O— and Y′ is NR^{v}, then R¹¹ attached to —O— is independently selected from the group consisting of —H, alkyl, optionally substituted aryl, optionally substituted heterocycloalkyl, optionally substituted CH₂-heterocycloakyl wherein the cyclic moiety contains a carbonate or thiocarbonate, optionally substituted -alkylaryl, —C(R^{z})₂OC(O)NR^{z}₂, —NR^{z}—C(O)—R^{y}, —C(R^{z})₂—OC(O)R^{y}, —C(R^{z})₂—O—C(O)OR^{y}, —C(R^{z})₂OC(O)SR^{y}, -alkyl-S—C(O)R^{y}, -alkyl-S—S-alkylhydroxy, and -alkyl-S—S—S-alkylhydroxy; and R¹¹ attached to —NR^{v}— is independently selected from the group consisting of H, —[C(R^{z})₂]_{q}—COOR^{y}, —C(R^{x})₂COOR^{y}, —[C(R^{z})₂]_{q}—C(O)SR^{y}, and -cycloalkylene-COOR^{y};
or when Y and Y′ are independently selected from —O— and NR^{v}, then together R¹¹ and R¹¹ are -alkyl-S—S-alkyl- to form a cyclic group, or together R¹¹ and R¹¹ are the group: 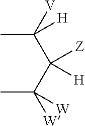
wherein:
V, W, and W′ are independently selected from the group consisting of hydrogen, optionally substituted alkyl, optionally substituted aralkyl, heterocycloalkyl, aryl, substituted aryl, heteroaryl, substituted heteroaryl, optionally substituted 1-alkenyl, and optionally substituted 1-alkynyl;
or together V and Z are connected via an additional 3-5 atoms to form a cyclic group containing 5-7 atoms, wherein 0-1 atoms are heteroatoms and the remaining atoms are carbon, substituted with hydroxy, acyloxy, alkylthiocarbonyloxy, alkoxycarbonyloxy, or aryloxycarbonyloxy attached to a carbon atom that is three atoms from both Y groups attached to the phosphorus;
or together V and Z are connected via an additional 3-5 atoms to form a cyclic group, wherein 0-1 atoms are heteroatoms and the remaining atoms are carbon, that is fused to an aryl group at the beta and gamma position to the Y attached to the phosphorus;
or together V and W are connected via an additional 3 carbon atoms to form an optionally substituted cyclic group containing 6 carbon atoms and substituted with one substituent selected from the group consisting of hydroxy, acyloxy, alkoxycarbonyloxy, alkylthiocarbonyloxy, and aryloxycarbonyloxy, attached to one of said carbon atoms that is three atoms from a Y attached to the phosphorus;
or together Z and W are connected via an additional 3-5 atoms to form a cyclic group, wherein 0-1 atoms are heteroatoms and the remaining atoms are carbon, and V must be aryl, substituted aryl, heteroaryl, or substituted heteroaryl;
or together W and W′ are connected via an additional 2-5 atoms to form a cyclic group, wherein 0-2 atoms are heteroatoms and the remaining atoms are carbon, and V must be aryl, substituted aryl, heteroaryl, or substituted heteroaryl;
Z is selected from the group consisting of —CHR^{z}OH, —CHR^{z}OC(O)R^{y}, —CHR^{z}OC(S)R^{y}, —CHR^{z}OC(S)OR^{y}, —CHR^{z}OC(O)SR^{y}, —CHR^{z}OCO₂R^{y}, —OR^{z}, —SR^{z}, —CHR^{z}N₃, —CH₂-aryl, —CH(aryl)OH, —CH(CH═CR^{z}₂)OH, —CH(C≡CR^{z})OH, —R^{z}, —NR^{z}₂, —OCOR^{y}, —OCO₂R^{y}, —SCOR^{y}, —SCO₂R^{y}, —NHCOR^{z}, —NHCO₂R^{y}, —CH₂NH-aryl, —(CH₂)q-OR^{z}, and —(CH₂)q-SR^{z};
q is an integer 2 or 3;
each R^{z} is selected from the group consisting of R^{y} and —H;
each R^{y} is selected from the group consisting of alkyl, aryl, heterocycloalkyl, and aralkyl;
each R^{x} is independently selected from the group consisting of —H, and alkyl, or together R^{x} and R^{x} form a cyclic alkyl group; and
each R^{v} is selected from the group consisting of —H, lower alkyl, acyloxyalkyl, alkoxycarbonyloxyalkyl, and lower acyl.

3. The method of claim 1, wherein the TR-β agonist is a compound having the structure of Formula (A): 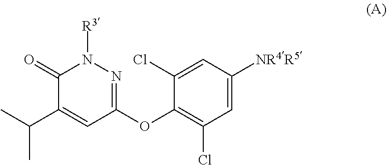
wherein
R^{3′} is H or CH₂R^{a′}, in which R^{a′} is hydroxyl, O-linked amino acid, —OP(O)(OH)₂ or OC(O)R^{b′}, R^{b′} being lower alkyl, alkoxy, alkyl acid, cycloalkyl, aryl, heteroaryl, or —(CH₂)ₙ-heteroaryl and n′ being 0 or 1;
R^{4′} is H, and R^{5′} is CH₂COOH, C(O)CO₂H, or an ester or amide thereof, or R⁴ and R⁵ together are —N═C(R^{c′})—C—(O)—NH—C(O)—; in which R^{c′} is H or cyano;
or pharmaceutically acceptable salts thereof.

4. The method of claim 3, wherein the TR-β agonist is 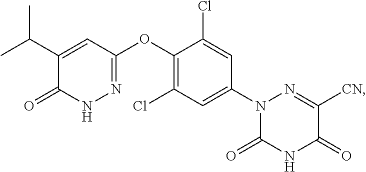 [SMILES: CC(C)c1cc(Oc2c(Cl)cc(-n3nc(C#N)c(=O)[nH]c3=O)cc2Cl)n[nH]c1=O]
or a pharmaceutically acceptable salt thereof.

5. The method of any one of claims 1-4 wherein the second pharmaceutical agent is a selected from the group consisting of peroxisome proliferator-activated receptor (PPAR) modulator, a bile acid receptor modulator, an anti-inflammatory compound, an antifibrotic compound, a GLP-1 (Glucagon-like peptide-1) agonist, and a metabolic modulator.

6. The method of claim 5, wherein the second pharmaceutical agent is a PPAR modulator.

7. The method of claim 6, wherein the PPAR modulator is: 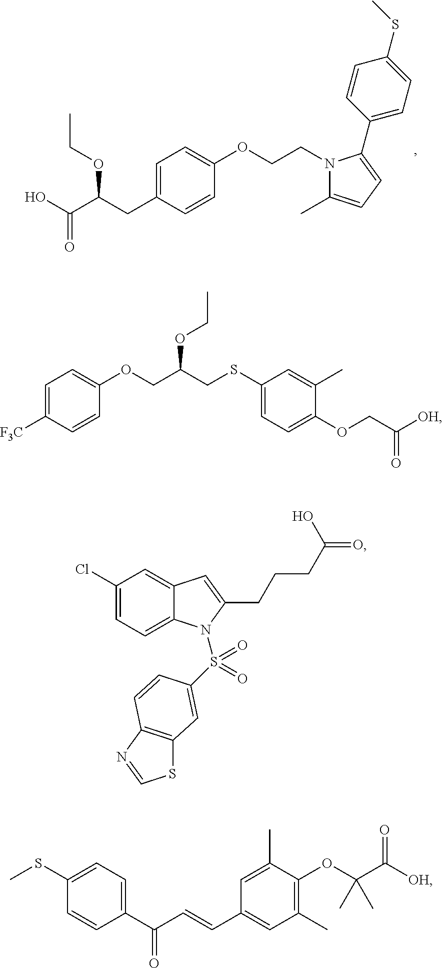 [SMILES: CCO[C@@H](Cc1ccc(OCCn2c(C)ccc2-c2ccc(SC)cc2)cc1)C(=O)O ; CCO[C@H](COc1ccc(C(F)(F)F)cc1)CSc1ccc(OCC(=O)O)c(C)c1 ; O=C(O)CCCc1cc2cc(Cl)ccc2n1S(=O)(=O)c1ccc2ncsc2c1 ; CSc1ccc(C(=O)/C=C/c2cc(C)c(OC(C)(C)C(=O)O)c(C)c2)cc1]
or a pharmaceutically acceptable salt thereof.

8. The method of claim 5, wherein the second pharmaceutical agent is a fibric acid derivative.

9. The method of claim 8, wherein the fibric acid derivative is fenofibrate, gemfibrozil, fenofibric acid, or clofibrate, or a pharmaceutically acceptable salt thereof.

10. The method of claim 5, wherein the second pharmaceutical agent is a bile acid receptor modulator.

11. The method of claim 10, wherein the bile acid receptor modulator is: 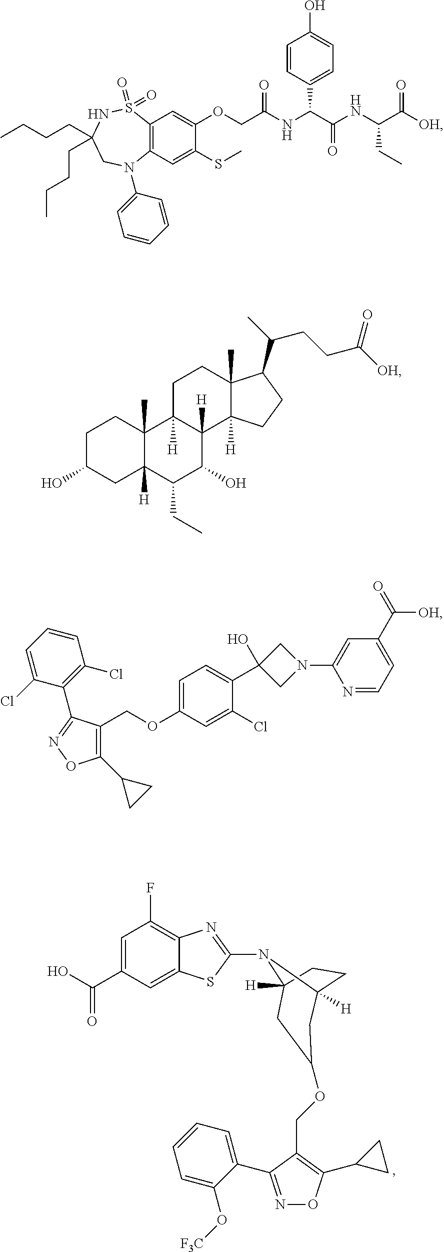
or a pharmaceutically acceptable salt thereof.

12. The method of claim 11, wherein the bile acid receptor modulator is

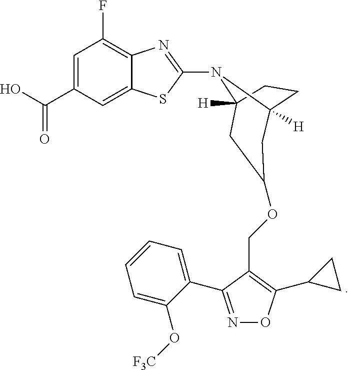 [SMILES: O=C(O)c1cc(F)c2nc(N3[C@@H]4CC[C@H]3CC(OCc3c(-c5ccccc5OC(F)(F)F)noc3C3CC3)C4)sc2c1]

13. The method of claim 5, wherein the second pharmaceutical agent is an anti-inflammatory compound.

14. The method of claim 13, wherein the anti-inflammatory compound is: 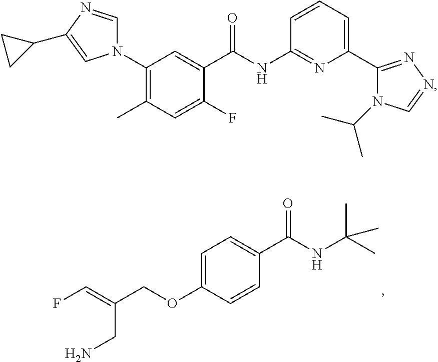 [SMILES: Cc1cc(F)c(C(=O)Nc2cccc(-c3nncn3C(C)C)n2)cc1-n1cnc(C2CC2)c1 ; CC(C)(C)NC(=O)c1ccc(OC/C(=C/F)CN)cc1]
IMM-124E or a pharmaceutically acceptable salt thereof.

15. The method of claim 5, wherein the second pharmaceutical agent is a GLP-1 agonist.

16. The method of claim 15, wherein the GLP-1 agonist is selected from dulaglutide, exenatide, liraglutide, albiglutide, lixisenatide, semaglutide, insulin glargine and

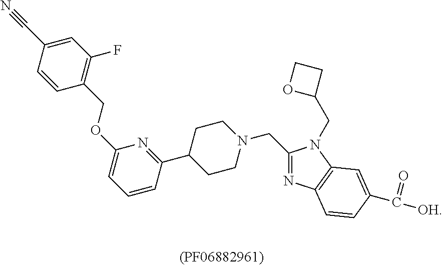 [SMILES: N#Cc1ccc(COc2cccc(C3CCN(Cc4nc5ccc(C(=O)O)cc5n4CC4CCO4)CC3)n2)c(F)c1]

17. The method of claim 16, wherein the GLP-1 agonist is semaglutide.

18. The method of claim 16, wherein the GLP-1 agoinst is liraglutide.

19. The method of claim 5, wherein the second pharmaceutical agent is an anti-fibrotic compound.

20. The method of claim 19, wherein the anti-fibrotic compound is: 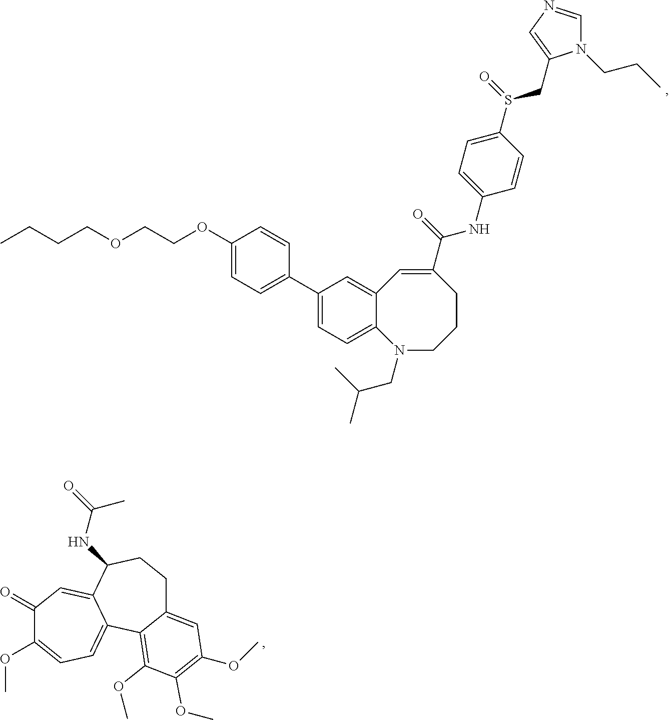 [SMILES: CCCCOCCOc1ccc(-c2ccc3c(c2)/C=C(/C(=O)Nc2ccc([S@@](=O)Cc4cncn4CCC)cc2)CCCN3CC(C)C)cc1 ; COc1cc2c(c(OC)c1OC)-c1ccc(OC)c(=O)cc1[C@@H](NC(C)=O)CC2]
or a pharmaceutically acceptable salt thereof.

21. The method of claim 5, wherein the second pharmaceutical agent is a metabolic modulator.

22. The method of claim 21, wherein the metabolic modulator is a thyroid hormone receptor agonist, a selective androgen receptor modulator, a mitochondrial membrane transport protein modulator, a selective estrogen receptor modulator, an inhibitor of stearoyl-CoA desaturase 1 (SCD1), an inhibitor of dipeptidyl peptidase 4 (DPP-4), an inhibitor of sodium glucose cotransporters 1 and/or 2, recombinant fibroblast growth factor 19 (FGF19) or engineered thereof, or recombinant fibroblast growth factor 21 (FGF21) or pegylated variants thereof.

23. The method of claim 21, wherein the metabolic modulator is: 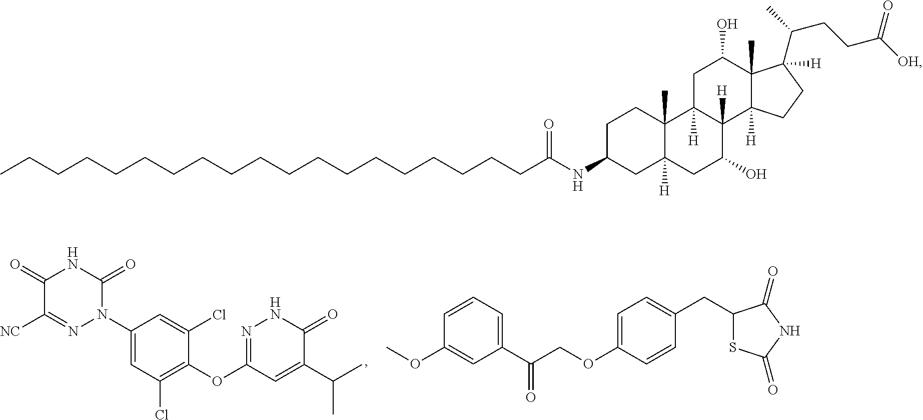
or a pharmaceutically acceptable salt thereof.

24. The method of claim 5, wherein the second pharmaceutical agent is a fish oil derivative.

25. The method of claim 24, wherein the fish oil derivative is an omega-3-fatty acid alkyl ester or an omega-3-fatty acid trigylyceride.

26. The method of claim 25, wherein the omega-3-fatty acid alkyl ester is an omega-3-fatty acid ethyl ester.

27. The method of claim 26, wherein the omega-3-fatty acid ethyl ester is ethyl (5Z,8Z,11Z,14Z,17Z)-eicosa-5,8,11,14,17-pentaenoate, ethyl (4Z,7Z,10Z,13Z,16Z,19Z)-docosa-4,7,10,13,16,19-hexaenoate, ethyl (7Z,10Z,13Z,16Z,19Z)-docosapentaenoate, ethyl hexadecatrienoate, α-linolenic acid ethyl ester, ethyl (6Z,9Z,12Z,15Z)-6,9,12,15-octadecatetraenoate, ethyl eicosatrienoate, ethyl eicosatetraenoate, ethyl heneicosapentaenoate, ethyl icosapentaenoate, ethyl heneicosapentaenoate, ethyl tetracosapentaenoate, or nisinic acid ethyl ester.

28. The method of any one of claims 1-27 wherein said fatty liver disease is selected from the group consisting of steatosis, non-alcoholic fatty liver disease, and non-alcoholic steatohepatitis.

29. A method of preventing, treating, or ameliorating one or more fatty liver diseases in a subject, comprising administering one or more compounds having a structure selected from the group consisting of: 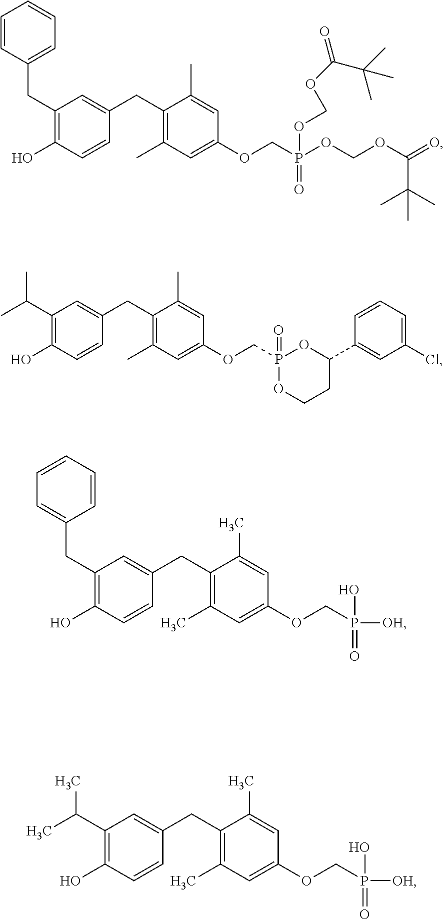
or pharmaceutically acceptable salts thereof to a subject in need thereof, in combination with a second pharmaceutical agent.

30. The method of claim 29 wherein the second pharmaceutical agent is selected from the group consisting of a peroxisome proliferator-activated receptor (PPAR) modulator, a bile acid receptor modulator, an anti-inflammatory compound, an antifibrotic compound, a GLP-1 (Glucagon-like peptide-1) agonist, and a metabolic modulator.

31. The method of claim 30, wherein the second pharmaceutical agent is a PPAR modulator.

32. The method of claim 31, wherein the PPAR modulator is: 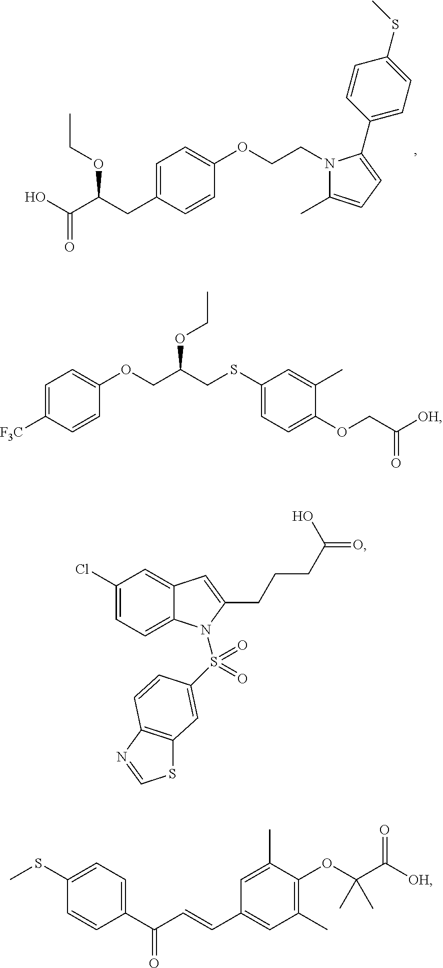 [SMILES: CCO[C@@H](Cc1ccc(OCCn2c(C)ccc2-c2ccc(SC)cc2)cc1)C(=O)O ; CCO[C@H](COc1ccc(C(F)(F)F)cc1)CSc1ccc(OCC(=O)O)c(C)c1 ; O=C(O)CCCc1cc2cc(Cl)ccc2n1S(=O)(=O)c1ccc2ncsc2c1 ; CSc1ccc(C(=O)/C=C/c2cc(C)c(OC(C)(C)C(=O)O)c(C)c2)cc1]
or a pharmaceutically acceptable salt thereof.

33. The method of claim 30, wherein the second pharmaceutical agent is a fibric acid derivative.

34. The method of claim 33, wherein the fibric acid derivative is fenofibrate, gemfibrozil, fenofibric acid, or clofibrate, or a pharmaceutically acceptable salt thereof.

35. The method of claim 30, wherein the second pharmaceutical agent is a bile acid receptor modulator.

36. The method of claim 33, wherein the bile acid receptor modulator is: 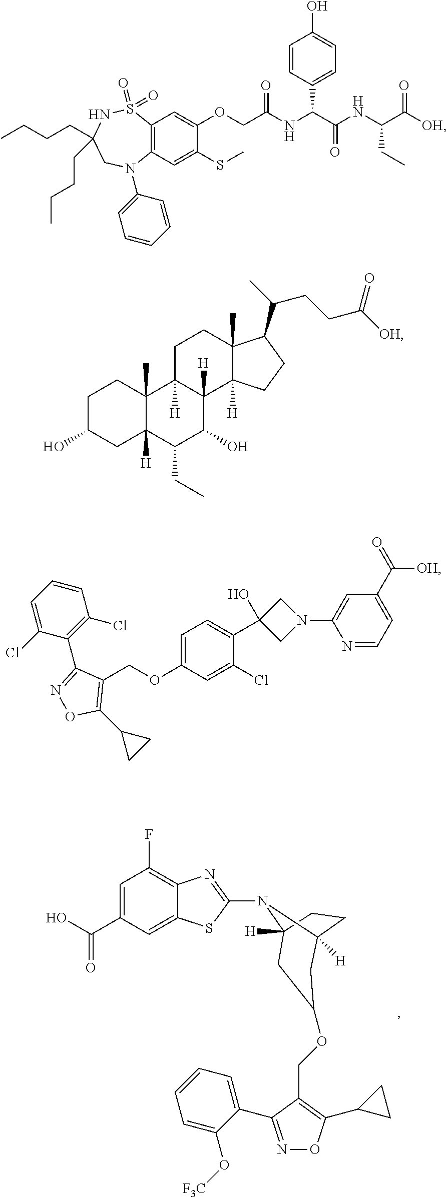
or a pharmaceutically acceptable salt thereof.

37. The method of claim 36, wherein the bile acid receptor modulator is

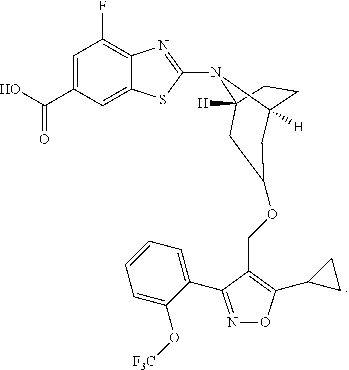 [SMILES: O=C(O)c1cc(F)c2nc(N3[C@@H]4CC[C@H]3CC(OCc3c(-c5ccccc5OC(F)(F)F)noc3C3CC3)C4)sc2c1]

38. The method of claim 30, wherein the second pharmaceutical agent is an anti-inflammatory compound.

39. The method of claim 38, wherein the anti-inflammatory compound is: 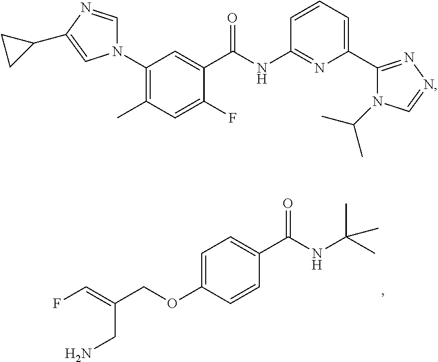 [SMILES: Cc1cc(F)c(C(=O)Nc2cccc(-c3nncn3C(C)C)n2)cc1-n1cnc(C2CC2)c1 ; CC(C)(C)NC(=O)c1ccc(OC/C(=C/F)CN)cc1]
IMM-124E or a pharmaceutically acceptable salt thereof.

40. The method of claim 30, wherein the second pharmaceutical agent is an anti-fibrotic compound.

41. The method of claim 40, wherein the anti-fibrotic compound is: 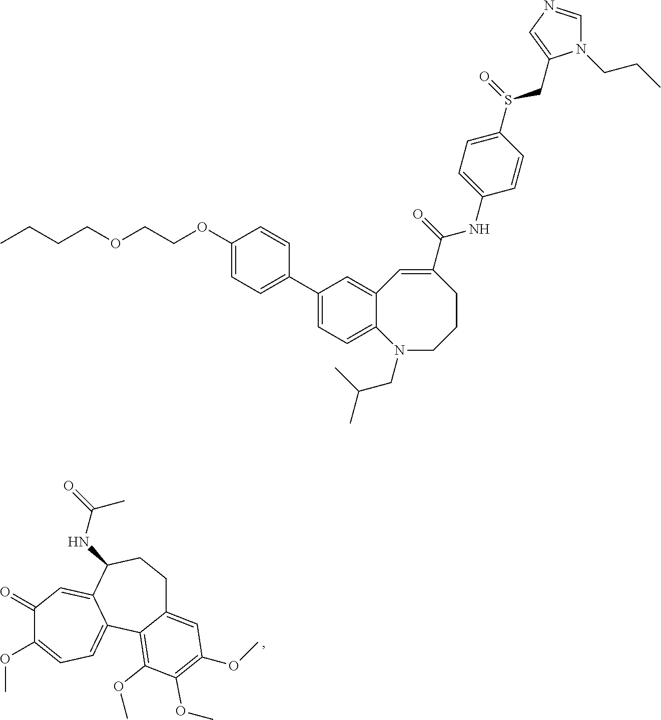 [SMILES: CCCCOCCOc1ccc(-c2ccc3c(c2)/C=C(/C(=O)Nc2ccc([S@@](=O)Cc4cncn4CCC)cc2)CCCN3CC(C)C)cc1 ; COc1cc2c(c(OC)c1OC)-c1ccc(OC)c(=O)cc1[C@@H](NC(C)=O)CC2]
or a pharmaceutically acceptable salt thereof.

42. The method of claim 30, wherein the second pharmaceutical agent is a metabolic modulator.

43. The method of claim 42, wherein the metabolic modulator is a thyroid hormone receptor agonist, a selective androgen receptor modulator, a mitochondrial membrane transport protein modulator, a selective estrogen receptor modulator, an inhibitor of stearoyl-CoA desaturase 1 (SCD1), an inhibitor of dipeptidyl peptidase 4 (DPP-4), an inhibitor of sodium glucose cotransporters 1 and/or 2, recombinant fibroblast growth factor 19 (FGF19) or engineered thereof, or recombinant fibroblast growth factor 21 (FGF21) or pegylated variants thereof.

44. The method of claim 42, wherein the metabolic modulator is: 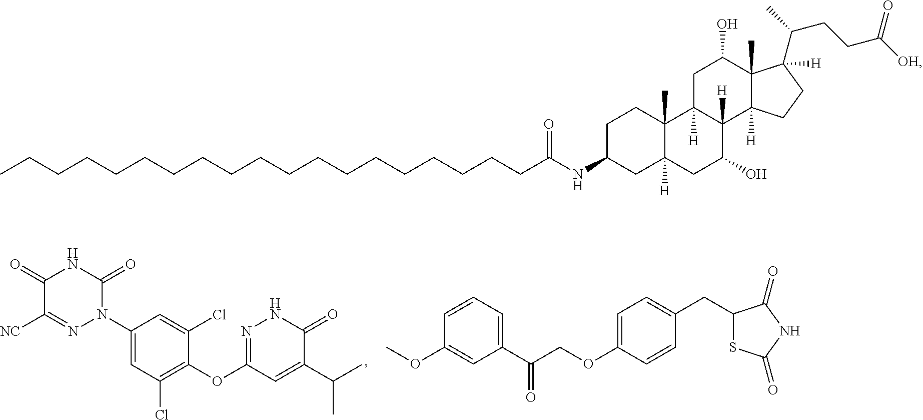
or a pharmaceutically acceptable salt thereof.

45. The method of claim 30, wherein the second pharmaceutical agent is a GLP-1 agonist.

46. The method of claim 45, wherein the GLP-1 agonist is selected from dulaglutide, exenatide, liraglutide, albiglutide, lixisenatide, semaglutide, insulin glargine, and

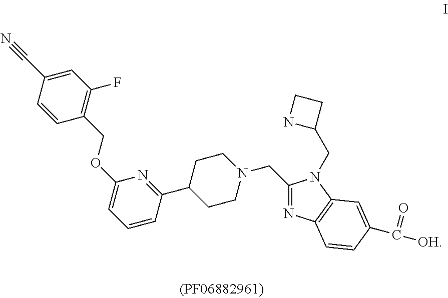 [SMILES: N#Cc1ccc(COc2cccc(C3CCN(Cc4nc5ccc(C(=O)O)cc5n4CC4CCN4)CC3)n2)c(F)c1]

47. The method of claim 46, wherein the GLP-1 agonist is semaglutide.

48. The method of claim 46, wherein the GLP-1 agoinst is liraglutide.

49. The method of claim 30, wherein the second pharmaceutical agent is a fish oil derivative.

50. The method of claim 49, wherein the fish oil derivative is an omega-3-fatty acid alkyl ester or an omega-3-fatty acid trigylyceride.

51. The method of claim 50, wherein the omega-3-fatty acid alkyl ester is an omega-3-fatty acid ethyl ester.

52. The method of claim 51, wherein the omega-3-fatty acid ethyl ester is ethyl (5Z,8Z,11Z,14Z,17Z)-eicosa-5,8,11,14,17-pentaenoate, ethyl (4Z,7Z,10Z,13Z,16Z,19Z)-docosa-4,7,10,13,16,19-hexaenoate, ethyl (7Z,10Z,13Z,16Z,19Z)-docosapentaenoate, ethyl hexadecatrienoate, α-linolenic acid ethyl ester, ethyl (6Z,9Z,12Z,15Z)-6,9,12,15-octadecatetraenoate, ethyl eicosatrienoate, ethyl eicosatetraenoate, ethyl heneicosapentaenoate, ethyl icosapentaenoate, ethyl heneicosapentaenoate, ethyl tetracosapentaenoate, or nisinic acid ethyl ester.

53. The method of any one of claims 29 to 52 wherein said wherein said fatty liver disease is selected from the group consisting of steatosis, non-alcoholic fatty liver disease, and non-alcoholic steatohepatitis.

54. The method of any one of claims 1-53, comprising administering a composition comprising one or more compounds of Formula I;
or one or more compounds selected from 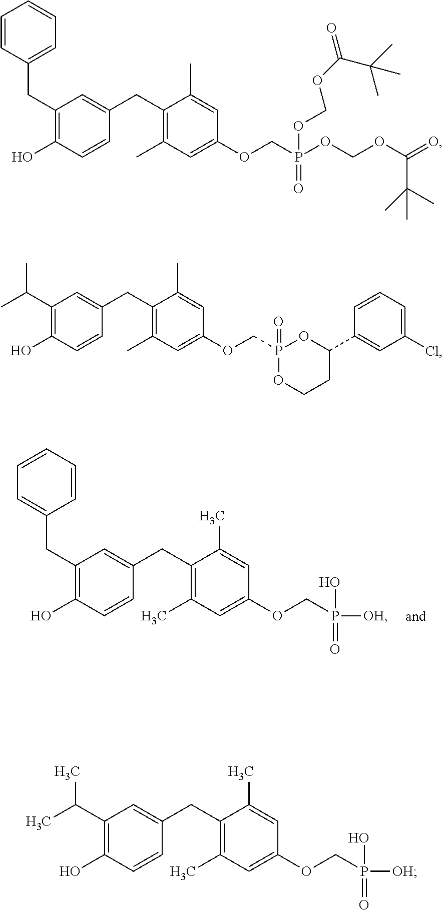
or one or more compounds having a structure of Formula (A): 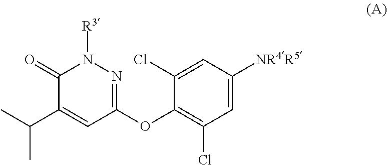
wherein
R^{3′} is H or CH₂R^{a′}, in which R^{a′} is hydroxyl, O-linked amino acid, —OP(O)(OH)₂ or OC(O)R^{b′}, R^{b′} being lower alkyl, alkoxy, alkyl acid, cycloalkyl, aryl, heteroaryl, or —(CH₂)_{n′}-heteroaryl and n′ being 0 or 1;
R^{4′} is H, and R^{5′} is CH₂COOH, C(O)CO₂H, or an ester or amide thereof, or R^{4′} and R^{5′} together are —N═C(R^{c′})—C—(O)—NH—C(O)—; in which R^{c′} is H or cyano;
or pharmaceutically acceptable salts thereof;
or 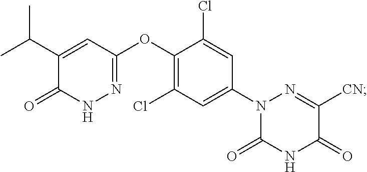 [SMILES: CC(C)c1cc(Oc2c(Cl)cc(-n3nc(C#N)c(=O)[nH]c3=O)cc2Cl)n[nH]c1=O]
and one or more pharmaceutically acceptable excipients.

55. The method of any one of claims 1-54 wherein said composition is formulated for oral, intravenous, intraarterial, intestinal, rectal, vaginal, nasal, pulmonary, topical, intradermal, transdermal, transbuccal, translingual, sublingual, or opthalmic administration, or any combination thereof.

56. The method of any one of claims 1-55, wherein the second pharmaceutical agent is administered sequentially or simultaneously.

57. The method of any one of claims 1-56 wherein said administration of said compound and said second pharmaceutical agent results in the prevention, treatment, or amelioration, of a fibrosis, fibrotic condition, or fibrotic symptom.

58. The method of any one of claims 1-57 wherein said administration of said compound and said second pharmaceutical agent results in the reduction in the amount of extracellular matrix proteins present in one or more tissues of said subject.

59. The method of any of claims 1-58 wherein said administration of said compound and said second pharmaceutical agent results in the reduction in the amount of collagen present in one or more tissues of said subject.

60. The method of claim 59 wherein said administration of said compound results in the reduction in the amount of Type I, Type Ia, or Type III collagen present in one or more tissues of said subject.

61. A pharmaceutical composition comprising at least one compound of Formula 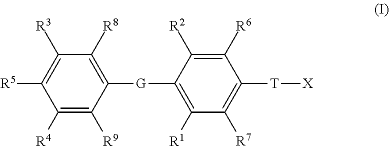
or a pharmaceutically acceptable salt thereof, wherein:
G is selected from the group consisting of —O—, —S—, —S(═O)—, —S(═O)₂—, —Se—, —CH₂—, —CF₂—, —CHF—, —C(O)—, —CH(OH)—, —CH(C₁-C₄ alkyl)-, —CH(C₁-C₄ alkoxy)-, —C(═CH₂)—, —NH—, and —N(C₁-C₄ alkyl)-;
T is selected from the group consisting of —(CR^{a}₂)ₖ—, —CR^{b}═CR^{b}—(CR^{a}₂)ₙ—, —(CR^{a}₂)ₙ—CR^{b}═CR^{b}—, —(CR^{a}₂)—CR^{b}═CR^{b}—(CR^{a}₂)—, —O(CR^{b}₂)(CR^{a}₂)ₙ—, —S(CR^{b}₂)(CR^{a}₂)ₙ—, N(R^{c})(CR^{b}₂)(CR^{a}₂)ₙ—, N(R^{b})C(O)(CR^{a}₂)ₙ, —C(O)(CR^{a}₂)ₘ—, —(CR^{a}₂)ₘC(O)—, —(CR^{a}₂)C(O)(CR^{a}₂)ₙ, —(CR^{a}₂)ₙC(O)(CR^{a}₂)—, and —C(O)NH(CR^{b}₂)(CR^{a}₂)ₚ—;
k is an integer from 1-4;
m is an integer from 0-3;
n is an integer from 0-2;
p is an integer from 0-1;
each R^{a} is independently selected from the group consisting of hydrogen, optionally substituted —C₁-C₄ alkyl, halogen, —OH, optionally substituted —O—C₁-C₄ alkyl, —OCF₃, optionally substituted —S—C₁-C₄ alkyl, —NR^{b}R^{c}, optionally substituted —C₂-C₄ alkenyl, and optionally substituted —C₂-C₄ alkynyl; with the proviso that when one R^{a} is attached to C through an O, S, or N atom, then the other R^{a} attached to the same C is a hydrogen, or attached via a carbon atom;
each R^{b} is independently selected from the group consisting of hydrogen and optionally substituted —C₁-C₄ alkyl;
each R^{c} is independently selected from the group consisting of hydrogen and optionally substituted —C₁-C₄ alkyl, optionally substituted —C(O)—C₁-C₄ alkyl, and —C(O)H;
R¹, and R² are each independently selected from the group consisting of halogen, optionally substituted —C₁-C₄ alkyl, optionally substituted —S—C₁-C₃ alkyl, optionally substituted —C₂-C₄ alkenyl, optionally substituted —C₂-C₄ alkynyl, —CF₃, —OCF₃, optionally substituted-O—C₁-C₃ alkyl, and cyano;
R⁶, R⁷, R⁸, and R⁹ are each independently selected from the group consisting of are each independently selected from the group consisting of hydrogen, halogen, optionally substituted —C C₁-C₄ alkyl, optionally substituted —S—C₁-C₃ alkyl, optionally substituted —C₂-C₄ alkenyl, optionally substituted —C₂-C₄ alkynyl, —CF₃, —OCF₃, optionally substituted-O—C₁-C₃ alkyl, and cyano; or R⁶ and T are taken together along with the carbons they are attached to form a ring of 5 to 6 atoms including 0 to 2 heteroatoms independently selected from —NRⁱ—, —O—, and —S—, with the proviso that when there are 2 heteroatoms in the ring and both heteroatoms are different than nitrogen then both heteroatoms have to be separated by at least one carbon atom; and X is attached to this ring by a direct bond to a ring carbon, or by —(CR^{a}₂)— or —C(O)— bonded to a ring carbon or a ring nitrogen;
Rⁱ is selected from the group consisting of hydrogen, —C(O)C₁-C₄ alkyl, —C₁-C₄ alkyl, and —C₁-C₄-aryl;
R³ and R⁴ are independently selected from the group consisting of hydrogen, halogen, —CF₃, —OCF₃, cyano, optionally substituted —C₁-C₁₂ alkyl, optionally substituted —C₂-C₁₂ alkenyl, optionally substituted —C₂-C₁₂ alkynyl, —SR^{d}, —S(═O)R^{e}, —S(═O)₂R^{e}, —S(═O)₂NR^{f}R^{g}, —C(O)OR^{h}, —C(O)R^{e}, —N(R^{b})C(O)NR^{f}R^{g}, —N(R^{b})S(═O)₂R^{e}, —N(R^{b})S(═O)₂NR^{f}R^{g}, and —NR^{f}R^{g};
each R^{d} is selected from the group consisting of optionally substituted —C₁-C₁₂ alkyl, optionally substituted —C₂-C₁₂ alkenyl, optionally substituted —C₂-C₁₂ alkynyl, optionally substituted —(CR^{b}₂)ₙ aryl, optionally substituted —(CR^{b}₂)ₙ cycloalkyl, optionally substituted —(CR^{b}₂)ₙ heterocycloalkyl, and —C(O)NR^{f}R^{g};
each R^{e} is selected from the group consisting of optionally substituted —C₁-C₁₂ alkyl, optionally substituted —C₂-C₁₂ alkenyl, optionally substituted —C₂-C₁₂ alkynyl, optionally substituted —(CR^{a}₂)ₙ aryl, optionally substituted —(CR^{a}₂)ₙ cycloalkyl, and optionally substituted —(CR^{a}₂)ₙ heterocycloalkyl;
R^{f} and R^{g} are each independently selected from the group consisting of hydrogen, optionally substituted —C₁-C₁₂ alkyl, optionally substituted —C₂-C₁₂ alkenyl, optionally substituted —C₂-C₁₂ alkynyl, optionally substituted —(CR^{b}₂)ₙ aryl, optionally substituted —(CR^{b}₂)ₙ cycloalkyl, and optionally substituted —(CR^{b}₂)ₙ heterocycloalkyl, or R^{f} and R^{g} may together form an optionally substituted heterocyclic ring, which may contain a second heterogroup selected from the group consisting of O, NR^{C}, and S, wherein said optionally substituted heterocyclic ring may be substituted with 0-4 substituents selected from the group consisting of optionally substituted —C₁-C₄ alkyl, —OR^{b}, oxo, cyano, —CF₃, optionally substituted phenyl, and —C(O)OR^{h};
each R^{h} is selected from the group consisting of optionally substituted —C₁-C₁₂ alkyl, optionally substituted —C₂-C₁₂ alkenyl, optionally substituted —C₂-C₁₂ alkynyl, optionally substituted —(CR^{b}₂)ₙ aryl, optionally substituted —(CR^{b}₂)ₙ cycloalkyl, and optionally substituted —(CR^{b}₂)ₙ heterocycloalkyl;
R⁵ is selected from the group consisting of —OH, optionally substituted —OC₁-C₆ alkyl, OC(O)R^{e}, —OC(O)OR^{h}, —F, —NHC(O)R^{e}, —NHS(═O)R^{e}, —NHS(═O)₂R^{e}, —NHC(═S)NH(R^{h}), and —NHC(O)NH(R^{h});
X is P(O)YR¹¹Y′R¹¹;
Y and Y′ are each independently selected from the group consisting of —O—, and —NR^{v}—; when Y and Y′ are —O—, R¹¹ attached to —O— is independently selected from the group consisting of —H, alkyl, optionally substituted aryl, optionally substituted heterocycloalkyl, optionally substituted CH₂-heterocycloakyl wherein the cyclic moiety contains a carbonate or thiocarbonate, optionally substituted -alkylaryl, —C(R^{z})₂OC(O)NR^{z}₂, —NR^{z}—C(O)—R^{y}, —C(R^{z})₂—OC(O)R^{y}, —C(R^{z})₂—O—C(O)OR^{y}, —C(R^{z})₂OC(O)SR^{y}, -alkyl-S—C(O)R^{y}, -alkyl-S—S-alkylhydroxy, and -alkyl-S—S—S-alkylhydroxy;
when Y and Y′ are —NR^{v}—, then R¹¹ attached to —NR^{v}— is independently selected from the group consisting of —H, —[C(R^{z})₂]_{q}—COOR^{y}, —C(R^{x})₂COOR^{Y}, —[C(R^{z})₂]_{q}—C(O)SR^{y}, and -cycloalkylene-COOR^{y};
when Y is —O— and Y′ is NR^{v}, then R¹¹ attached to —O— is independently selected from the group consisting of —H, alkyl, optionally substituted aryl, optionally substituted heterocycloalkyl, optionally substituted CH₂-heterocycloakyl wherein the cyclic moiety contains a carbonate or thiocarbonate, optionally substituted -alkylaryl, —C(R^{z})₂OC(O)NR^{z}₂, —NR^{z}—C(O)—R^{y}, —C(R^{z})₂—OC(O)R^{y}, —C(R^{z})₂—O—C(O)OR^{y}, —C(R^{z})₂OC(O)SR^{y}, -alkyl-S—C(O)R^{y}, -alkyl-S—S-alkylhydroxy, and -alkyl-S—S—S-alkylhydroxy; and R¹¹ attached to —NR^{v}— is independently selected from the group consisting of H, —[C(R^{z})₂]_{q}—COOR^{y}, —C(R^{x})₂COOR^{y}, —[C(R^{z})₂]_{q}—C(O)SR^{y}, and -cycloalkylene-COOR^{y};
or when Y and Y′ are independently selected from —O— and NR^{v}, then together R¹¹ and R¹¹ are -alkyl-S—S-alkyl- to form a cyclic group, or together R¹¹ and R¹¹ are the group: 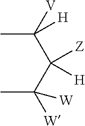
wherein:
V, W, and W′ are independently selected from the group consisting of hydrogen, optionally substituted alkyl, optionally substituted aralkyl, heterocycloalkyl, aryl, substituted aryl, heteroaryl, substituted heteroaryl, optionally substituted 1-alkenyl, and optionally substituted 1-alkynyl;
or together V and Z are connected via an additional 3-5 atoms to form a cyclic group containing 5-7 atoms, wherein 0-1 atoms are heteroatoms and the remaining atoms are carbon, substituted with hydroxy, acyloxy, alkylthiocarbonyloxy, alkoxycarbonyloxy, or aryloxycarbonyloxy attached to a carbon atom that is three atoms from both Y groups attached to the phosphorus;
or together V and Z are connected via an additional 3-5 atoms to form a cyclic group, wherein 0-1 atoms are heteroatoms and the remaining atoms are carbon, that is fused to an aryl group at the beta and gamma position to the Y attached to the phosphorus;
or together V and W are connected via an additional 3 carbon atoms to form an optionally substituted cyclic group containing 6 carbon atoms and substituted with one substituent selected from the group consisting of hydroxy, acyloxy, alkoxycarbonyloxy, alkylthiocarbonyloxy, and aryloxycarbonyloxy, attached to one of said carbon atoms that is three atoms from a Y attached to the phosphorus;
or together Z and W are connected via an additional 3-5 atoms to form a cyclic group, wherein 0-1 atoms are heteroatoms and the remaining atoms are carbon, and V must be aryl, substituted aryl, heteroaryl, or substituted heteroaryl;
or together W and W′ are connected via an additional 2-5 atoms to form a cyclic group, wherein 0-2 atoms are heteroatoms and the remaining atoms are carbon, and V must be aryl, substituted aryl, heteroaryl, or substituted heteroaryl;
Z is selected from the group consisting of —CHR^{z}OH, —CHR^{z}OC(O)R^{y}, —CHR^{z}OC(S)R^{y}, —CHR^{z}OC(S)OR^{y}, —CHR^{z}OC(O)SR^{y}, —CHR^{z}OCO₂R^{y}, —OR^{z}, —SR^{z}, —CHR^{z}N₃, —CH₂-aryl, —CH(aryl)OH, —CH(CH═CR^{z}₂)OH, —CH(C≡CR^{z})OH, —R^{z}, —NR^{z}₂, —OCOR^{y}, —OCO₂R^{y}, —SCOR^{y}, —SCO₂R^{y}, —NHCOR^{z}, —NHCO₂R^{y}, —CH₂NH-aryl, —(CH₂)q-OR^{z}, and —(CH₂)q-SR^{z};
q is an integer 2 or 3;
each R^{z} is selected from the group consisting of R^{y} and —H;
each R^{y} is selected from the group consisting of alkyl, aryl, heterocycloalkyl, and aralkyl;
each R^{x} is independently selected from the group consisting of —H, and alkyl, or together R^{x} and R^{x} form a cyclic alkyl group; and
each R^{v} is selected from the group consisting of —H, lower alkyl, acyloxyalkyl, alkoxycarbonyloxyalkyl, and lower acyl;
or one or more compounds selected from 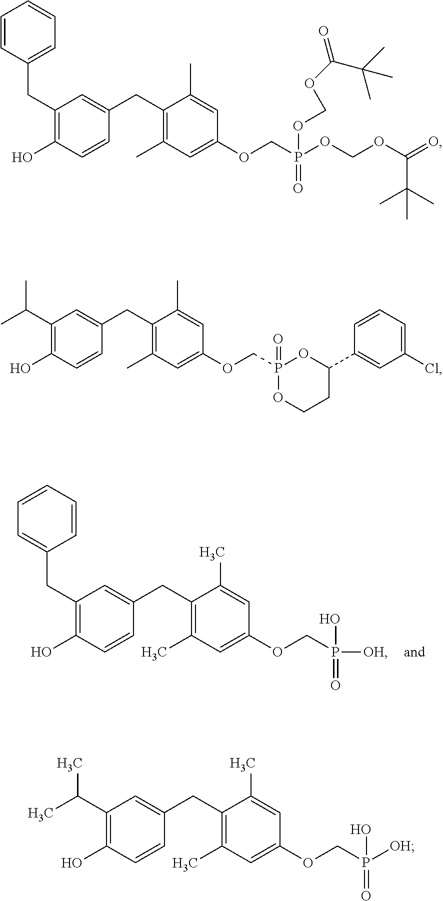
or one or more compounds having a structure of Formula (A): 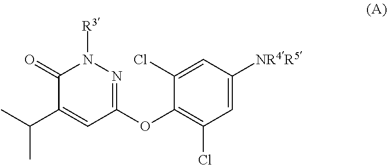
wherein
R^{3′} is H or CH₂R^{a′}, in which R^{a′} is hydroxyl, O-linked amino acid, —OP(O)(OH)₂ or OC(O)R^{b′}, R^{b′} being lower alkyl, alkoxy, alkyl acid, cycloalkyl, aryl, heteroaryl, or —(CH₂)_{n′}-heteroaryl and n′ being 0 or 1;
R^{4′} is H, and R^{5′} is CH₂COOH, C(O)CO₂H, or an ester or amide thereof, or R^{4′} and R^{5′} together are —N═C(R^{c′})—C—(O)—NH—C(O)—; in which R^{c′} is H or cyano;
or pharmaceutically acceptable salts thereof;
or 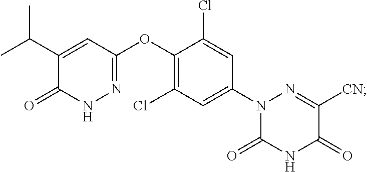 [SMILES: CC(C)c1cc(Oc2c(Cl)cc(-n3nc(C#N)c(=O)[nH]c3=O)cc2Cl)n[nH]c1=O]
and one or more pharmaceutically acceptable excipients; in combination with a second pharmaceutical agent.

62. The pharmaceutical composition of claim 61 wherein the second pharmaceutical agent is selected from the group consisting of a peroxisome proliferator-activated receptor (PPAR) modulator, a bile acid receptor modulator, an anti-inflammatory compound, an antifibrotic compound, a GLP-1 (Glucagon-like peptide-1) agonist, and a metabolic modulator.

63. The pharmaceutical composition of claim 62, wherein the second pharmaceutical agent is a PPAR modulator.

64. The pharmaceutical composition of claim 63, wherein the PPAR modulator is: 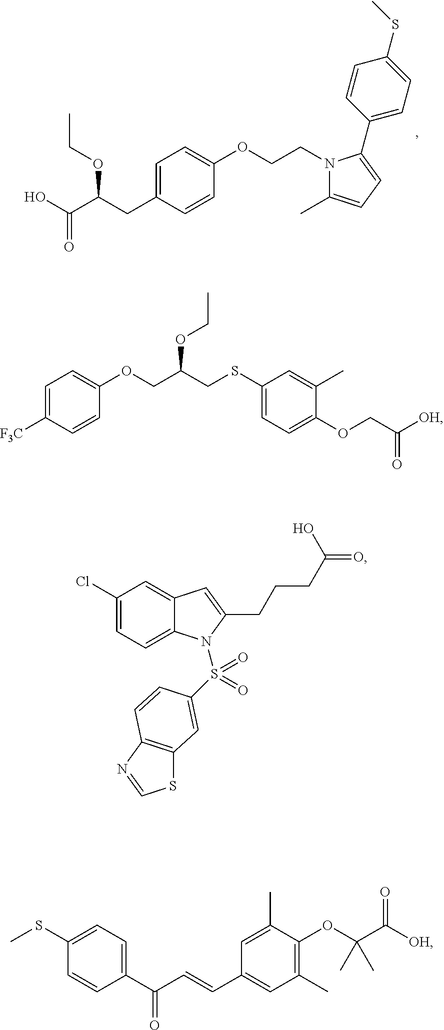 [SMILES: CCO[C@@H](Cc1ccc(OCCn2c(C)ccc2-c2ccc(SC)cc2)cc1)C(=O)O ; CCO[C@H](COc1ccc(C(F)(F)F)cc1)CSc1ccc(OCC(=O)O)c(C)c1 ; O=C(O)CCCc1cc2cc(Cl)ccc2n1S(=O)(=O)c1ccc2ncsc2c1 ; CSc1ccc(C(=O)/C=C/c2cc(C)c(OC(C)(C)C(=O)O)c(C)c2)cc1]
or a pharmaceutically acceptable salt thereof.

65. The pharmaceutical composition of claim 62, wherein the second pharmaceutical agent is a fibric acid derivative.

66. The pharmaceutical composition of claim 65, wherein the fibric acid derivative is fenofibrate, gemfibrozil, fenofibric acid, or clofibrate, or a pharmaceutically acceptable salt thereof.

67. The pharmaceutical composition of claim 62, wherein the second pharmaceutical agent is a bile acid receptor modulator.

68. The pharmaceutical composition of claim 67, wherein the bile acid receptor modulator is: 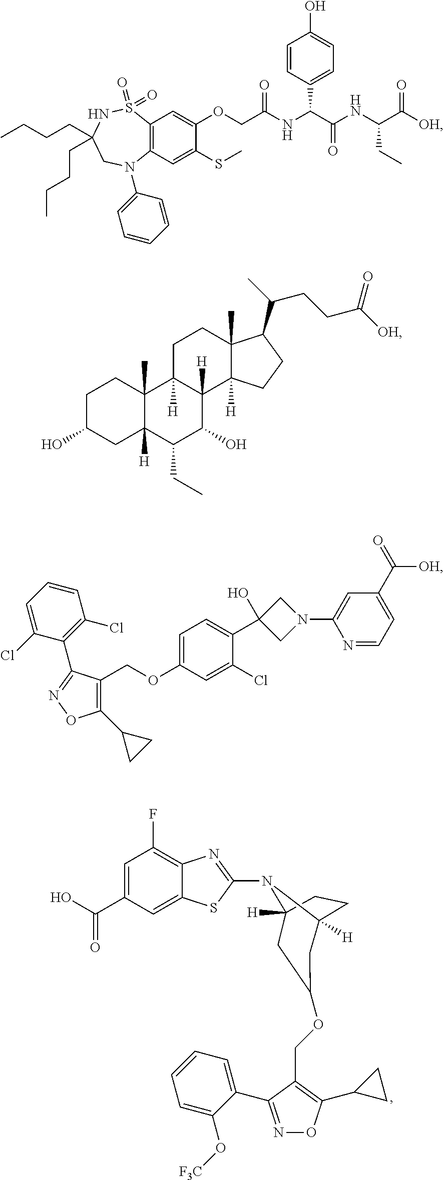
or a pharmaceutically acceptable salt thereof.

69. The pharmaceutical composition of claim 68, wherein the bile acid receptor modulator is

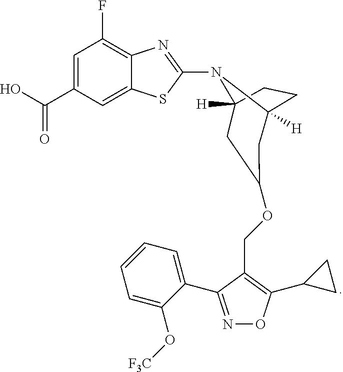 [SMILES: O=C(O)c1cc(F)c2nc(N3[C@@H]4CC[C@H]3CC(OCc3c(-c5ccccc5OC(F)(F)F)noc3C3CC3)C4)sc2c1]

70. The pharmaceutical composition of claim 62, wherein the second pharmaceutical agent is an anti-inflammatory compound.

71. The pharmaceutical composition of claim 70, wherein the anti-inflammatory compound is: 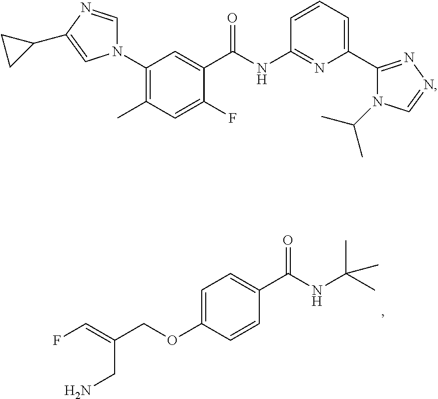 [SMILES: Cc1cc(F)c(C(=O)Nc2cccc(-c3nncn3C(C)C)n2)cc1-n1cnc(C2CC2)c1 ; CC(C)(C)NC(=O)c1ccc(OC/C(=C/F)CN)cc1]
IMM-124E or a pharmaceutically acceptable salt thereof.

72. The pharmaceutical composition of claim 62, wherein the second pharmaceutical agent is a GLP-1 agonist.

73. The pharmaceutical composition of claim 72, wherein the GLP-1 agonist is selected from dulaglutide, exenatide, liraglutide, albiglutide, lixisenatide, semaglutide, insulin glargine, and

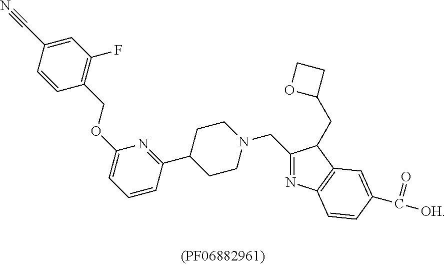 [SMILES: N#Cc1ccc(COc2cccc(C3CCN(CC4=Nc5ccc(C(=O)O)cc5C4CC4CCO4)CC3)n2)c(F)c1]

74. The method of claim 72, wherein the GLP-1 agonist is semaglutide.

75. The method of claim 72, wherein the GLP-1 agoinst is liraglutide.

76. The pharmaceutical composition of claim 62, wherein the second pharmaceutical agent is an anti-fibrotic compound.

77. The pharmaceutical composition of claim 76, wherein the anti-fibrotic compound is: 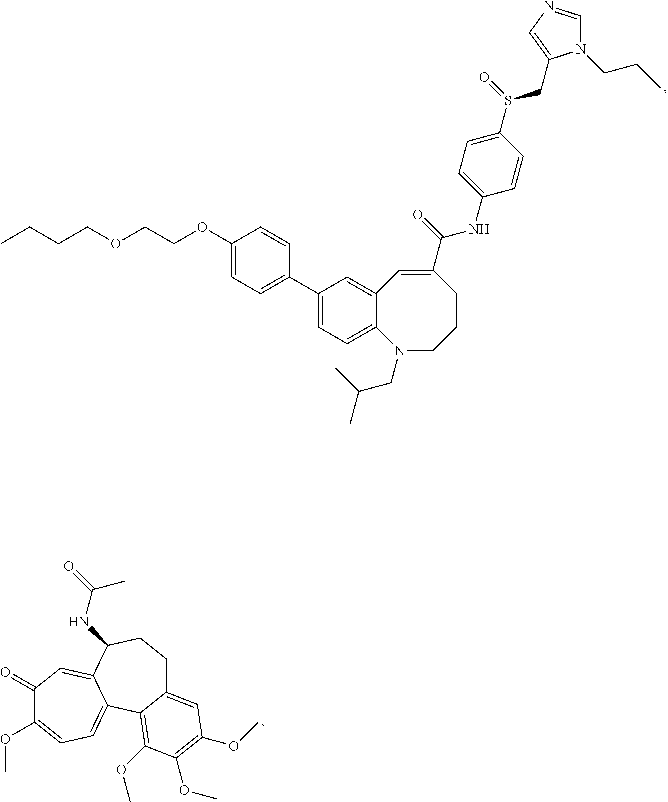 [SMILES: CCCCOCCOc1ccc(-c2ccc3c(c2)/C=C(/C(=O)Nc2ccc([S@@](=O)Cc4cncn4CCC)cc2)CCCN3CC(C)C)cc1 ; COc1cc2c(c(OC)c1OC)-c1ccc(OC)c(=O)cc1[C@@H](NC(C)=O)CC2]
or a pharmaceutically acceptable salt thereof.

78. The pharmaceutical composition of claim 62, wherein the second pharmaceutical agent is a metabolic modulator.

79. The pharmaceutical composition of claim 78, wherein the metabolic modulator is a thyroid hormone receptor agonist, a selective androgen receptor modulator, a mitochondrial membrane transport protein modulator, a selective estrogen receptor modulator, an inhibitor of stearoyl-CoA desaturase 1 (SCD1), an inhibitor of dipeptidyl peptidase 4 (DPP-4), an inhibitor of sodium glucose cotransporters 1 and/or 2, recombinant fibroblast growth factor 19 (FGF19) or engineered thereof, or recombinant fibroblast growth factor 21 (FGF21) or pegylated variants thereof.

80. The pharmaceutical composition of claim 78, wherein the metabolic modulator is: 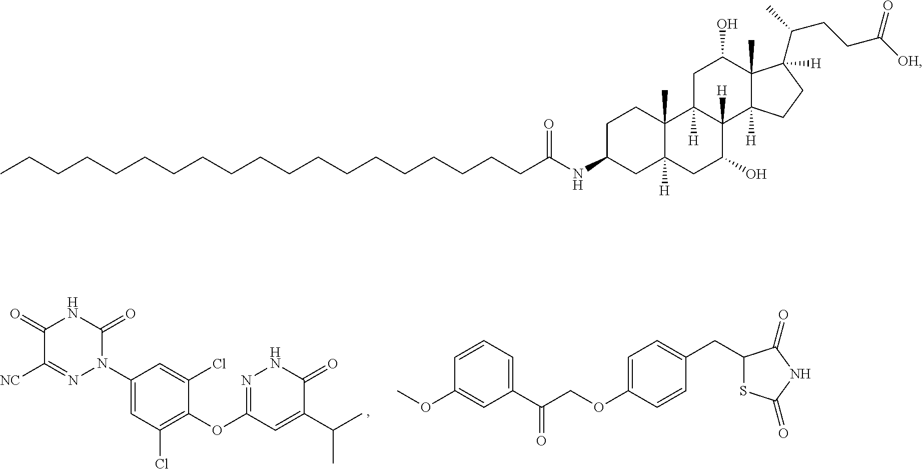
or a pharmaceutically acceptable salt thereof.

81. The pharmaceutical composition of claim 62, wherein the second pharmaceutical agent is a fish oil derivative.

82. The pharmaceutical composition of claim 81, wherein the fish oil derivative is an omega-3-fatty acid alkyl ester or an omega-3-fatty acid trigylyceride.

83. The pharmaceutical composition of claim 82, wherein the omega-3-fatty acid alkyl ester is an omega-3-fatty acid ethyl ester.

84. The pharmaceutical composition of claim 83, wherein the omega-3-fatty acid ethyl ester is ethyl (5Z,8Z,11Z,14Z,17Z)-eicosa-5,8,11,14,17-pentaenoate, ethyl (4Z,7Z,10Z,13Z,16Z,19Z)-docosa-4,7,10,13,16,19-hexaenoate, ethyl (7Z,10Z,13Z,16Z,19Z)-docosapentaenoate, ethyl hexadecatrienoate, α-linolenic acid ethyl ester, ethyl (6Z,9Z,12Z,15Z)-6,9,12,15-octadecatetraenoate, ethyl eicosatrienoate, ethyl eicosatetraenoate, ethyl heneicosapentaenoate, ethyl icosapentaenoate, ethyl heneicosapentaenoate, ethyl tetracosapentaenoate, or nisinic acid ethyl ester.

85. The pharmaceutical composition of any one of claims 61-84, further comprising one or more pharmaceutically acceptable excipients.
